(19) United States
(12) Patent Application Publication
(10) Pub. No.: US 2025/0082512 A1
(43) Pub. Date: Mar. 13, 2025

(54) NEGATIVE PRESSURE WOUND TREATMENT APPARATUSES AND METHODS WITH INTEGRATED ELECTRONICS

(71) Applicant: Smith & Nephew PLC, Watford (GB)

(72) Inventors: Victoria Beadle, Hull (GB); Frederick Jethro Harrison, Cambridge (GB); Mark Richard Hesketh, Royston (GB); William Kelbie, Inverness (GB); Damyn Musgrave, Cottenham (GB); Joseph William Robinson, Papworth Everard (GB); Daniel Lee Steward, Hull (GB); Grant West, East Hyde (GB)

(21) Appl. No.: 18/893,837

(22) Filed: Sep. 23, 2024

Related U.S. Application Data
(63) Continuation-in-part of application No. 16/338,386, filed on Mar. 29, 2019.
(63) Continuation-in-part of application No. 18/096,937, filed on Jan. 13, 2023, now Pat. No. 12,127,919.
Application No. 18/096,937 is a continuation of application No. 16/338,400, filed on Mar. 29, 2019, now Pat. No. 11,564,847.
(63) Continuation-in-part of application No. 18/202,559, filed on May 26, 2023, now Pat. No. 12,097,097.
Application No. 18/202,559 is a continuation of application No. 16/645,769, filed on Mar. 9, 2020, now Pat. No. 11,701,265.
(60) Provisional application No. 62/402,382, filed on Sep. 30, 2016.
(60) Provisional application No. 62/402,298, filed on Sep. 30, 2016.
(60) Provisional application No. 62/402,146, filed on Sep. 30, 2016.
(60) Provisional application No. 62/558,263, filed on Sep. 13, 2017.
(60) Provisional application No. 62/402,551, filed on Sep. 30, 2016.
(60) Provisional application No. 62/558,264, filed on Sep. 13, 2017.

(51) Int. Cl. A61F 13/05; A61F 13/00; A61F 13/0203; A61F 13/0246; A61M 1/00; F21Y 115/10

(52) U.S. Cl. CPC A61F 13/05; A61F 13/022; A61M 1/962; A61F 2013/00174; A61F 13/0253; F21Y 2115/10

(57) ABSTRACT

Disclosed embodiments relate to apparatuses and methods for wound treatment. In some embodiments, a wound dressing apparatus includes a wound contact layer, a first area and a second area over the wound contact layer, and a cover layer configured to cover the wound contact layer, the first area, and the second area. In some embodiments, a wound dressing apparatus includes a wound contact layer, a spacer layer, an absorbent layer positioned on the spacer layer, an electronics unit including a negative pressure source and/or electronic components, wherein the absorbent layer includes a recess configured to receive the electronics unit and the absorbent layer is configured to be in fluid communication with the electronics unit, and a cover layer configured to cover and form a seal over the wound contact layer, the spacer layer, the absorbent layer, and the electronics unit.

### CROSS-REFERENCE TO RELATED APPLICATIONS

Any and all applications for which a foreign or domestic priority claim is identified in the Application Data Sheet as filed with the present application are hereby incorporated by reference under 37 C.F.R. 1.57.

This application is a continuation-in-part of co-pending U.S. application Ser. No. 16/338,386, filed Mar. 29, 2019, which is a National Stage Entry of International Application No. PCT/EP2017/074755, filed Sep. 29, 2017, which claims priority to U.S. Provisional Application Nos. 62/402,382, filed Sep. 30, 2016, 62/402,298, filed Sep. 30, 2016, and 62/402,146, filed Sep. 30, 2016.

This application is also a continuation-in-part of co-pending U.S. application Ser. No. 18/096,937, filed Jan. 13, 2023, which is a continuation of U.S. application Ser. No. 16/338,400, filed Mar. 29, 2019 and issued as U.S. Pat. No. 11,564,847, which is a National Stage Entry of International Application No. PCT/EP2017/074764, filed Sep. 29, 2017, which claims priority to U.S. Provisional Application Nos. 62/558,263, filed Sep. 13, 2017, and 62/402,551, filed Sep. 30, 2016.

This application is also a continuation-in-part of co-pending U.S. application Ser. No. 18/202,559, filed May 26, 2023, which is a continuation of U.S. application Ser. No. 16/645,769, filed Mar. 9, 2020 and issued as U.S. Pat. No. 11,701,265, which is a National Stage Entry of International Application No. PCT/EP2018/074694, filed Sep. 13, 2018, which claims priority to U.S. Provisional Application No. 62/558,264, filed Sep. 13, 2017.

The disclosures of all these prior applications are hereby incorporated herein by reference in their entireties and are to be to be considered a part of this specification.

### BACKGROUND

### Technical Field

Embodiments described herein relate to apparatuses, systems, and methods the treatment of wounds, for example using dressings in combination with negative pressure wound therapy.

### Description of the Related Art

The treatment of open or chronic wounds that are too large to spontaneously close or otherwise fail to heal by means of applying negative pressure to the site of the wound is well known in the art. Negative pressure wound therapy (NPWT) systems currently known in the art commonly involve placing a cover that is impermeable or semi-permeable to fluids over the wound, using various means to seal the cover to the tissue of the patient surrounding the wound, and connecting a source of negative pressure (such as a vacuum pump) to the cover in a manner so that negative pressure is created and maintained under the cover. It is believed that such negative pressures promote wound healing by facilitating the formation of granulation tissue at the wound site and assisting the body's normal inflammatory process while simultaneously removing excess fluid, which may contain adverse cytokines and/or bacteria. However, further improvements in NPWT are needed to fully realize the benefits of treatment.

Many different types of wound dressings are known for aiding in NPWT systems. These different types of wound dressings include many different types of materials and layers, for example, gauze, pads, foam pads or multi-layer wound dressings. One example of a multi-layer wound dressing is the PICO dressing, available from Smith & Nephew, which includes a superabsorbent layer beneath a backing layer to provide a canister-less system for treating a wound with NPWT. The wound dressing may be sealed to a suction port providing connection to a length of tubing, which may be used to pump fluid out of the dressing and/or to transmit negative pressure from a pump to the wound dressing.

Prior art dressings for use in negative pressure such as those described above have included a negative pressure source located in a remote location from the wound dressing. Negative pressure sources located remote from the wound dressing have to be held by or attached to the user or other pump support mechanism. Additionally, a tubing or connector is required to connect the remote negative pressure source to the wound dressing. The remote pump and tubing can be cumbersome and difficult to hide in or attach to patient clothing. Depending on the location of the wound dressing, it can be difficult to comfortably and conveniently position the remote pump and tubing. When used, wound exudate may soak into the dressing, and the moisture from the wound has made it difficult to incorporate electronic components into the dressing.

### SUMMARY

Embodiments of the present disclosure relate to apparatuses and methods for wound treatment. Some of the wound treatment apparatuses described herein comprise a negative pressure source or a pump system for providing negative pressure to a wound. Wound treatment apparatuses may also comprise wound dressings that may be used in combination with the negative pressure sources and pump assemblies described herein. In some embodiments, a negative pressure source is incorporated into a wound dressing apparatus so that the wound dressing and the negative pressure source are part of an integral or integrated wound dressing structure that applies the wound dressing and the negative pressure source simultaneously to a patient's wound. The negative pressure source and/or electronic components may be positioned between a wound contact layer and a cover layer of the wound dressing. An electronics assembly can be incorporated into the absorbent material of the dressing and maintain conformability of the dressing. These and other embodiments as described herein are directed to overcoming particular challenges involved with incorporating a negative pressure source and/or electronic components into a wound dressing.

According to one embodiment, a wound dressing apparatus can comprise a wound contact layer configured to be positioned in contact with a wound, a first area over the wound contact layer can comprise a spacer layer and an absorbent layer over the spacer layer, a second area over the wound contact layer can comprise an electronics cassette, the electronics cassette can comprise a negative pressure source and/or electronic components surrounded by a casing, wherein the first area is positioned adjacent to the second area, wherein the casing is configured to surround the negative pressure source and/or electronic components and to allow fluid communication between the first area and second area, and a cover layer configured to cover and form a seal over the wound contact layer, the first area, and the second area.

The wound dressing apparatus of the preceding paragraph or in other embodiments can include one or more of the following features. The electronic components can comprise one or more of a power source, a flexible circuit board, a sensor, a switch, and/or a light or LED indicator. The wound dressing apparatus can further comprise a negative pressure source inlet protection mechanism and a negative pressure source outlet or exhaust. The negative pressure source outlet or exhaust can comprise an antibacterial membrane and/or a non-return valve. The cover layer can comprise an aperture over the outlet or exhaust. The negative pressure source inlet protection mechanism can comprise a hydrophobic material configured to prevent fluid from entering the negative pressure source. The electronics cassette can comprise one or more slits, grooves or recesses in the casing, wherein the slits, grooves or recesses are configured to provide one or more hinge points in the casing and increase flexibility of the electronics cassette. The spacer layer can extend within both the first area and the second area and the electronics cassette is provided over the spacer layer. The negative pressure source and/or electronic components can be encapsulated in a hydrophobic coating.

According to another embodiment, a wound dressing apparatus can comprise a wound contact layer configured to be positioned in contact with a wound, a first area over the wound contact layer can comprise a spacer layer and an absorbent layer over the spacer layer, a second area over the wound contact layer comprising an electronics unit, the electronics unit comprising a negative pressure source and/or electronic components, wherein the first area is positioned adjacent to the second area, wherein the second area can comprise a cradle configured to allow fluid communication between the first area and second area, wherein the cradle can comprise recesses configured to receive a portion of the electronics unit, and a cover layer configured to cover and form a seal over the wound contact layer, the first area, and the second area.

The wound dressing apparatus of the preceding paragraph or in other embodiments can include one or more of the following features. The cradle can comprise a hydrophilic material. The wound dressing apparatus wherein at least a portion of the second area can overlap a portion of the first area. The cradle can comprise a recess configured to receive a portion of the absorbent layer. The electronic components can comprise one or more of a power source, a flexible circuit board, a sensor, a switch, and/or a light or LED indicator. The wound dressing apparatus can further comprise a negative pressure source inlet protection mechanism and a negative pressure source outlet or exhaust. The negative pressure source outlet or exhaust can comprise an antibacterial membrane and/or a non-return valve. The cover layer can comprise an aperture over the outlet or exhaust. The negative pressure source inlet protection mechanism can comprise a hydrophobic material configured to prevent fluid from entering the negative pressure source. The negative pressure source and/or electronic components can be encapsulated in a hydrophobic coating.

According to another embodiment, a wound dressing apparatus can comprise a wound contact layer comprising a proximal wound-facing face and a distal face, wherein the wound-facing face is configured to be positioned in contact with a wound, a spacer layer comprising a proximal wound-facing face and a distal face, the spacer layer positioned over the distal face of the wound contact layer, a first area over the spacer layer comprising an absorbent layer, a second area over the spacer layer comprising an electronics unit, the electronics unit comprising a negative pressure source and/or electronic components, wherein the first area is positioned adjacent to the second area, wherein the second area over the spacer layer comprises a cradle configured to allow fluid communication between the first area and second area, wherein the cradle comprises recesses configured to receive the electronics unit, and a cover layer configured to cover and form a seal over the wound contact layer, the spacer layer, the first area, and the second area.

The wound dressing apparatus of the preceding paragraph or in other embodiments can include one or more of the following features. The cradle can comprise a hydrophilic material. The electronic components can comprise one or more of a power source, a flexible circuit board, a sensor, a switch, and/or a light or LED indicator. The wound dressing apparatus can further comprise a negative pressure source inlet protection mechanism and a negative pressure source outlet or exhaust. The negative pressure source outlet or exhaust can comprise an antibacterial membrane and/or a non-return valve. The cover layer can comprise an aperture over the outlet or exhaust. The negative pressure source inlet protection mechanism can comprise a hydrophobic material configured to prevent fluid from entering the negative pressure source.

According to another embodiment, a wound dressing apparatus can comprise a wound contact layer comprising a proximal wound-facing face and a distal face, wherein the proximal wound-facing face is configured to be positioned in contact with a wound, a spacer layer comprising a proximal wound-facing face and a distal face, the spacer layer positioned over the distal face of the wound contact layer, an absorbent layer comprising a proximal wound-facing face and a distal face, the absorbent layer positioned on the distal face of the spacer layer, an electronics unit comprising a negative pressure source and/or electronic components, a cradle positioned on the distal face of the absorbent layer and configured to allow fluid communication between the absorbent layer and the electronics unit, wherein the cradle comprises recesses configured to receive the electronics unit, and a cover layer configured to cover and form a seal over the wound contact layer, the spacer layer, the absorbent layer, the cradle and electronics unit.

The wound dressing apparatus of the preceding paragraph or in other embodiments can include one or more of the following features. The cradle can comprise a hydrophilic material. The electronic components can comprise one or more of a power source, a flexible circuit board, a sensor, a switch, and/or a light or LED indicator. The wound dressing apparatus can further comprise a negative pressure source inlet protection mechanism and a negative pressure source outlet or exhaust. The negative pressure source outlet or exhaust can comprise an antibacterial membrane and/or a non-return valve. The cover layer can comprise an aperture over the outlet or exhaust. The negative pressure source inlet protection mechanism can comprise a hydrophobic material configured to prevent fluid from entering the negative pressure source.

According to another embodiment, a wound dressing apparatus can comprise a wound contact layer comprising a proximal wound-facing face and a distal face, wherein the wound-facing face is configured to be positioned in contact with a wound, a first area over the distal face of the wound contact layer comprising a spacer layer, and an absorbent layer over the spacer layer, a second area over the distal face of the wound contact layer comprising an electronics unit, the electronics unit comprising a negative pressure source and/or electronic components, wherein the first area is positioned adjacent to the second area, wherein the second area comprises a cradle positioned directly on the wound contact layer, wherein the cradle is configured to allow fluid communication between the first area and second area, and wherein the cradle comprises recesses configured to receive the electronics unit, and a cover layer configured to cover and form a seal over the wound contact layer, the first area, and the second area.

The wound dressing apparatus of the preceding paragraph or in other embodiments can include one or more of the following features. The cradle can comprise a hydrophilic material. The electronic components can comprise one or more of a power source, a flexible circuit board, a sensor, a switch, and/or a light or LED indicator. The wound dressing apparatus can further comprise a negative pressure source inlet protection mechanism and a negative pressure source outlet or exhaust. The negative pressure source outlet or exhaust can comprise an antibacterial membrane and/or a non-return valve. The cover layer can comprise an aperture over the outlet or exhaust. The negative pressure source inlet protection mechanism can comprise a hydrophobic material configured to prevent fluid from entering the negative pressure source.

Embodiments of the present disclosure relate to apparatuses and methods for wound treatment. Some of the wound treatment apparatuses described herein comprise a negative pressure source or a pump system for providing negative pressure to a wound. Wound treatment apparatuses may also comprise wound dressings that may be used in combination with the negative pressure sources and pump assemblies described herein. In some embodiments, a negative pressure source is incorporated into a wound dressing apparatus so that the wound dressing and the negative pressure source are part of an integral or integrated wound dressing structure that applies the wound dressing and the negative pressure source simultaneously to a patient's wound. The negative pressure source and/or electronic components may be positioned between a wound contact layer and a cover layer of the wound dressing. An electronics assembly can be incorporated into the absorbent material of the dressing to prevent pooling of wound exudate and maintain conformability of the dressing. These and other embodiments as described herein are directed to overcoming particular challenges involved with incorporating a negative pressure source and/or electronic components into a wound dressing.

According to one embodiment, a wound dressing apparatus can comprise a wound contact layer, the wound contact layer can comprise a proximal wound-facing face and a distal face, wherein the proximal wound-facing face is configured to be positioned in contact with a wound, a spacer layer comprising a proximal wound-facing face and a distal face, the spacer layer positioned over the distal face of the wound contact layer, an absorbent layer positioned on the distal face of the spacer layer, an electronics unit comprising a negative pressure source and/or electronic components, wherein the absorbent layer comprises a recess configured to receive the electronics unit and the absorbent layer is configured to be in fluid communication with the electronics unit and a cover layer configured to cover and form a seal over the wound contact layer, the spacer layer, the absorbent layer, and the electronics unit.

The wound dressing apparatus of the preceding paragraph or in other embodiments can include one or more of the following features. The electronic components can comprise one or more of a power source, a flexible circuit board, a sensor, a switch, and/or a light or LED indicator. The wound dressing further can comprise a negative pressure source inlet protection mechanism and a negative pressure source outlet or exhaust. The negative pressure source outlet or exhaust can comprises an antibacterial membrane and/or a non-return valve. The cover layer can comprise an aperture over the outlet or exhaust. The negative pressure source inlet protection mechanism can comprise a hydrophobic material configured to prevent fluid from entering the negative pressure source.

Any of the features, components, or details of any of the arrangements or embodiments disclosed in this application, including without limitation any of the pump embodiments and any of the negative pressure wound therapy embodiments disclosed below, are interchangeably combinable with any other features, components, or details of any of the arrangements or embodiments disclosed herein to form new arrangements and embodiments.

Embodiments of the present disclosure relate to apparatuses and methods for wound treatment. Some of the wound treatment apparatuses described herein comprise a negative pressure source or a pump system for providing negative pressure to a wound. Wound treatment apparatuses may also comprise wound dressings that may be used in combination with the negative pressure sources and pump assemblies described herein. In some embodiments, a negative pressure source is incorporated into a wound dressing apparatus so that the wound dressing and the negative pressure source are part of an integral or integrated wound dressing structure that applies the wound dressing and the negative pressure source simultaneously to a patient's wound. The negative pressure source and/or electronic components may be positioned between a wound contact layer and a cover layer of the wound dressing. An electronics assembly can be incorporated into the absorbent material of the dressing to prevent pooling of wound exudate and maintain conformability of the dressing. These and other embodiments as described herein are directed to overcoming particular challenges involved with incorporating a negative pressure source and/or electronic components into a wound dressing.

According to one embodiment, a wound dressing apparatus can comprise a wound contact layer comprising a proximal wound-facing face and a distal face, wherein the proximal wound-facing face is configured to be positioned in contact with a wound, at least one absorbent layer over the wound contact layer, an electronics unit comprising a negative pressure source unit comprising a negative pressure source, inlet protection mechanism, and an outlet or exhaust mechanism, a plurality of sensors positioned on a printed circuit board, wherein the inlet protection mechanism comprises a first recess configured to be in fluid communication with a first sensor on the printed circuit board and the outlet or exhaust mechanism comprises a second recess configured to be in fluid communication with a second sensor on the printed circuit board, and wherein the at least one absorbent layer is configured to be in fluid communication with the electronics unit; and a cover layer configured to cover and form a seal over the wound contact layer, the at least one absorbent layer, and the electronics unit.

The wound dressing apparatus of the preceding paragraph or in other embodiments can include one or more of the following features. The first recess can be positioned over the first sensor, wherein the perimeter of the first recess comprises a first gasket configured to seal the perimeter of the first recess in the inlet protection mechanism to the printed circuit board surrounding the first sensor. The second recess can be positioned over the second sensor, wherein the perimeter of the second recess comprises a second gasket configured to seal the perimeter of the second recess of the outlet or exhaust mechanism to the printed circuit board surrounding the second sensor. The outlet or exhaust mechanism can comprises at least one vent aperture and the printed circuit board can comprise at least one vent hole in the printed circuit board, wherein the at least one vent aperture of the outlet or exhaust mechanism is configured to be in fluid communication with the at least one vent hole of the printed circuit board. The second gasket can comprise an aperture configured to provide fluid communication between a vent hole of the at least one vent hole of the printed circuit board and a vent aperture of the at least one vent aperture of the outlet or exhaust mechanism through the second gasket. The at least one vent aperture of the outlet or exhaust mechanism can comprise an antibacterial membrane and/or a non-return valve. The cover layer can comprise an aperture over the at least one vent aperture. The electronics unit can comprise one or more power sources. The at least one absorbent layer can comprise one or more recesses configured to receive the electronics unit. The wound dressing can further comprising a transmission layer comprising a proximal wound-facing face and a distal face, the transmission layer can be positioned over the distal face of the wound contact layer. The at least one absorbent layer can comprise a first absorbent layer comprising a proximal wound-facing face and a distal face, the first absorbent layer can be positioned on the distal face of the transmission layer and a second absorbent comprising a proximal wound-facing face and a distal face, the second absorbent layer can be positioned on the distal face of the first absorbent layer. The wound dressing can further comprising an overlay layer comprising a proximal wound-facing face and a distal face, the overlay layer can be positioned over the distal face of the second absorbent layer, wherein the overlay layer comprises a larger perimeter than a perimeter of the transmission layer and the first and second absorbent layer. The electronic unit can comprise a switch. The electronic unit can comprise a light or LED indicator.

Any of the features, components, or details of any of the arrangements or embodiments disclosed in this application, including without limitation any of the pump embodiments and any of the negative pressure wound therapy embodiments disclosed below, are interchangeably combinable with any other features, components, or details of any of the arrangements or embodiments disclosed herein to form new arrangements and embodiments.

### BRIEF DESCRIPTION OF THE DRAWINGS

FIGS. 1A-1B illustrate a wound dressing apparatus incorporating the pump and/or other electronic components within the wound dressing and offset from the absorbent layer;

FIGS. 2, 3A-3D, and 4A-4B illustrate embodiments of the electronics unit 67 that can be incorporated into a wound dressing;

FIGS. 5A-5E illustrate embodiments of components of the electronics cassette;

FIGS. 6A-6E illustrate an embodiment of the flexible circuit board for use in the electronics cassette;

FIGS. 7A-7D illustrate embodiments of electronic cassettes with two pump exhausts in various configurations and shapes;

FIGS. 8A-8C illustrate embodiments of electronic cassettes with one pump exhaust in various shapes;

FIGS. 9A-9D illustrate various embodiments of the electronic cassette with various configurations for components on the electronic cassettes;

FIG. 10 illustrates an embodiment of the electronics cassette;

FIGS. 11-16 illustrate an assembly process for the electronics unit of the electronics cassette;

FIG. 17 illustrates an embodiment of on/off switch for use with a wound dressing with integrated electronics;

FIG. 18 illustrates an embodiment of a wound dressing incorporating an electronics unit within the dressing;

FIG. 19A illustrates an embodiment of the electronics unit;

FIG. 19B illustrates an embodiment of the cradle that can be within, adjacent to, and/or above the absorbent dressing material;

FIG. 19C illustrates an embodiment of the electronics unit incorporated into the cradle;

FIGS. 20A-20B illustrate an embodiment of a wound dressing incorporating the electronics unit and electronics cradle adjacent to and at least partially overlapping the absorbent pad;

FIGS. 21A-21F illustrate an embodiment of a wound dressing incorporating an electronics unit resting in a cradle;

FIGS. 22A-22C illustrate another embodiment of a wound dressing incorporating an electronics unit and cradle in the wound dressing;

FIG. 23 illustrates an absorbent layer provided over the entire length of the spacer layer;

FIG. 24 illustrates an embodiment of a wound dressing incorporating an electronics unit resting in a cradle;

FIGS. 25A-25B illustrate embodiments of an assembled wound dressing with the dressing layers enclosed between a wound contact layer and a cover layer; and

FIG. 26 illustrates an embodiment of an assembled wound dressing.

FIG. 27A-27C illustrate a wound dressing apparatus incorporating the pump and/or other electronic components within the wound dressing and offset from the absorbent layer;

FIG. 28A illustrates an embodiment of the electronics unit;

FIGS. 28B-28C illustrate embodiments of the pump and electronics unit;

FIG. 29 illustrates an embodiment of a wound dressing incorporating an electronics unit within the dressing;

FIGS. 30A-30C illustrate an embodiment of a wound dressing incorporating an electronics unit in the absorbent layer;

FIGS. 31A-31B illustrate an embodiment of a wound dressing incorporating an electronics unit;

FIG. 32 illustrates an embodiment of wound dressing layers incorporating the electronic components within the wound dressing;

FIGS. 33A-33C illustrate embodiments of individual layers of a wound dressing;

FIGS. 34A-34F illustrate embodiments of layers of the wound dressing incorporating an electronics assembly within the dressing;

FIG. 35 illustrates a cross sectional layout of the material layers of the wound dressing incorporating an electronics assembly within the dressing

FIGS. 36A-36C and 37A-37B illustrate embodiments of components of an electronics unit including a printed circuit board, the negative pressure source, and one or more power sources;

FIG. 38 illustrates an embodiment of a pump assembly incorporating adhesive gaskets;

FIG. 39 illustrates an embodiment of a pump inlet protection mechanism; and

FIG. 40 illustrates an embodiment of a pump outlet mechanism.

### DETAILED DESCRIPTION

Embodiments disclosed herein relate to apparatuses and methods of treating a wound with reduced pressure, including a source of negative pressure and wound dressing components and apparatuses. The apparatuses and components comprising the wound overlay and packing materials, if any, are sometimes collectively referred to herein as dressings.

It will be appreciated that throughout this specification reference is made to a wound. It is to be understood that the term wound is to be broadly construed and encompasses open and closed wounds in which skin is torn, cut or punctured or where trauma causes a contusion, or any other superficial or other conditions or imperfections on the skin of a patient or otherwise that benefit from reduced pressure treatment. A wound is thus broadly defined as any damaged region of tissue where fluid may or may not be produced. Examples of such wounds include, but are not limited to, abdominal wounds or other large or incisional wounds, either as a result of surgery, trauma, sterniotomies, fasciotomies, or other conditions, dehisced wounds, acute wounds, chronic wounds, subacute and dehisced wounds, traumatic wounds, flaps and skin grafts, lacerations, abrasions, contusions, burns, diabetic ulcers, pressure ulcers, stoma, surgical wounds, trauma and venous ulcers or the like.

It will be understood that embodiments of the present disclosure are generally applicable to use in topical negative pressure (“TNP”) therapy systems. Briefly, negative pressure wound therapy assists in the closure and healing of many forms of “hard to heal” wounds by reducing tissue oedema; encouraging blood flow and granular tissue formation; removing excess exudate and may reduce bacterial load (and thus infection risk). In addition, the therapy allows for less disturbance of a wound leading to more rapid healing. TNP therapy systems may also assist on the healing of surgically closed wounds by removing fluid and by helping to stabilize the tissue in the apposed position of closure. A further beneficial use of TNP therapy can be found in grafts and flaps where removal of excess fluid is important and close proximity of the graft to tissue is required in order to ensure tissue viability.

As is used herein, reduced or negative pressure levels, such as −X mmHg, represent pressure levels relative to normal ambient atmospheric pressure, which can correspond to 760 mmHg (or 1 atm, 29.93 inHg, 101.325 kPa, 14.696 psi, etc.). Accordingly, a negative pressure value of −X mmHg reflects absolute pressure that is X mmHg below 760 mmHg or, in other words, an absolute pressure of (760−X) mmHg. In addition, negative pressure that is “less” or “smaller” than X mmHg corresponds to pressure that is closer to atmospheric pressure (e.g., −40 mmHg is less than −60 mmHg). Negative pressure that is “more” or “greater” than −X mmHg corresponds to pressure that is further from atmospheric pressure (e.g., −80 mmHg is more than −60 mmHg). In some embodiments, local ambient atmospheric pressure is used as a reference point, and such local atmospheric pressure may not necessarily be, for example, 760 mmHg.

The negative pressure range for some embodiments of the present disclosure can be approximately −80 mmHg, or between about −20 mmHg and −200 mmHg. Note that these pressures are relative to normal ambient atmospheric pressure, which can be 760 mmHg. Thus, −200 mmHg would be about 560 mmHg in practical terms. In some embodiments, the pressure range can be between about −40 mmHg and −150 mmHg. Alternatively a pressure range of up to −75 mmHg, up to −80 mmHg or over −80 mmHg can be used. Also in other embodiments a pressure range of below −75 mmHg can be used. Alternatively, a pressure range of over approximately −100 mmHg, or even −150 mmHg, can be supplied by the negative pressure apparatus.

In some embodiments of wound closure devices described herein, increased wound contraction can lead to increased tissue expansion in the surrounding wound tissue. This effect may be increased by varying the force applied to the tissue, for example by varying the negative pressure applied to the wound over time, possibly in conjunction with increased tensile forces applied to the wound via embodiments of the wound closure devices. In some embodiments, negative pressure may be varied over time for example using a sinusoidal wave, square wave, and/or in synchronization with one or more patient physiological indices (e.g., heartbeat). Examples of such applications where additional disclosure relating to the preceding may be found include U.S. Pat. No. 8,235,955, titled “Wound treatment apparatus and method,” issued on Aug. 7, 2012; and U.S. Pat. No. 7,753,894, titled “Wound cleansing apparatus with stress,” issued Jul. 13, 2010. The disclosures of both of these patents are hereby incorporated by reference in their entirety.

International Application PCT/GB2012/000587, titled “WOUND DRESSING AND METHOD OF TREATMENT” and filed on Jul. 12, 2012, and published as WO 2013/007973 A2 on Jan. 17, 2013, is an application, hereby incorporated and considered to be part of this specification, that is directed to embodiments, methods of manufacture, and wound dressing components and wound treatment apparatuses that may be used in combination or in addition to the embodiments described herein. Additionally, embodiments of the wound dressings, wound treatment apparatuses and methods described herein may also be used in combination or in addition to those described in U.S. Provisional Application No. 61/650,904, filed May 23, 2012, titled “APPARATUSES AND METHODS FOR NEGATIVE PRESSURE WOUND THERAPY,” International Application No. PCT/IB2013/001469, filed May 22, 2013, titled “APPARATUSES AND METHODS FOR NEGATIVE PRESSURE WOUND THERAPY,” and published as WO 2013/175306 on Nov. 28, 2013, U.S. patent application Ser. No. 14/418,874, filed Jan. 30, 2015, published as U.S. Publication No. 2015/0216733, published Aug. 6, 2015, titled “WOUND DRESSING AND METHOD OF TREATMENT,” U.S. patent application Ser. No. 14/418,908, filed Jan. 30, 2015, published as U.S. Publication No. 2015/0190286, published Jul. 9, 2015, titled “WOUND DRESSING AND METHOD OF TREATMENT,” U.S. patent application Ser. No. 14/658,068, filed Mar. 13, 2015, U.S. Application No. 2015/0182677, published Jul. 2, 2015, titled “WOUND DRESSING AND METHOD OF TREATMENT,” the disclosures of which are hereby incorporated by reference in their entireties. Embodiments of the wound dressings, wound treatment apparatuses and methods described herein may also be used in combination or in addition to those described in U.S. patent application Ser. No. 13/092,042, filed Apr. 21, 2011, published as US2011/0282309, titled “WOUND DRESSING AND METHOD OF USE,” and which is hereby incorporated by reference in its entirety, including further details relating to embodiments of wound dressings, the wound dressing components and principles, and the materials used for the wound dressings.

Embodiments of the wound dressings, wound treatment apparatuses and methods described herein relating to wound dressings with electronics incorporated into the dressing may also be used in combination or in addition to those described in International Application No. PCT/EP2017/055225, filed Mar. 6, 2017, titled “WOUND TREATMENT APPARATUSES AND METHODS WITH NEGATIVE PRESSURE SOURCE INTEGRATED INTO WOUND DRESSING,” and which is hereby incorporated by reference in its entirety herein, including further details relating to embodiments of wound dressings, the wound dressing components and principles, and the materials used for the wound dressings.

Disclosed embodiments relate to apparatuses and methods for wound treatment. In some embodiments, a wound dressing apparatus can comprise a wound contact layer, a first area and a second area over the wound contact layer, and a cover layer configured to cover the wound contact layer, the first area, and the second area. The first area can comprise a spacer layer and an absorbent layer over the spacer layer. The second area can comprise an electronics cassette or cradle comprising a negative pressure source and/or electronic components.

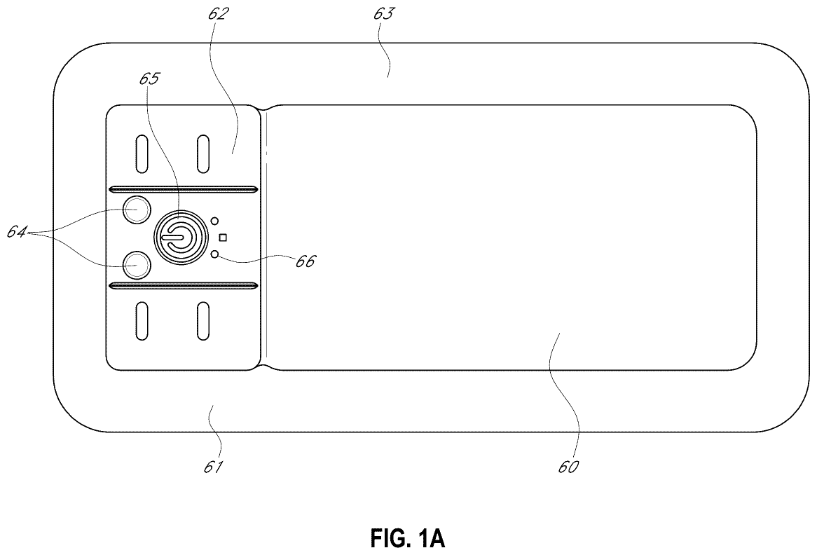

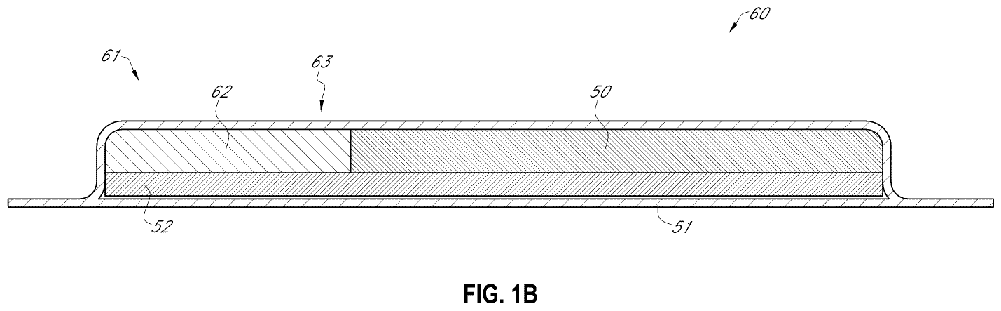

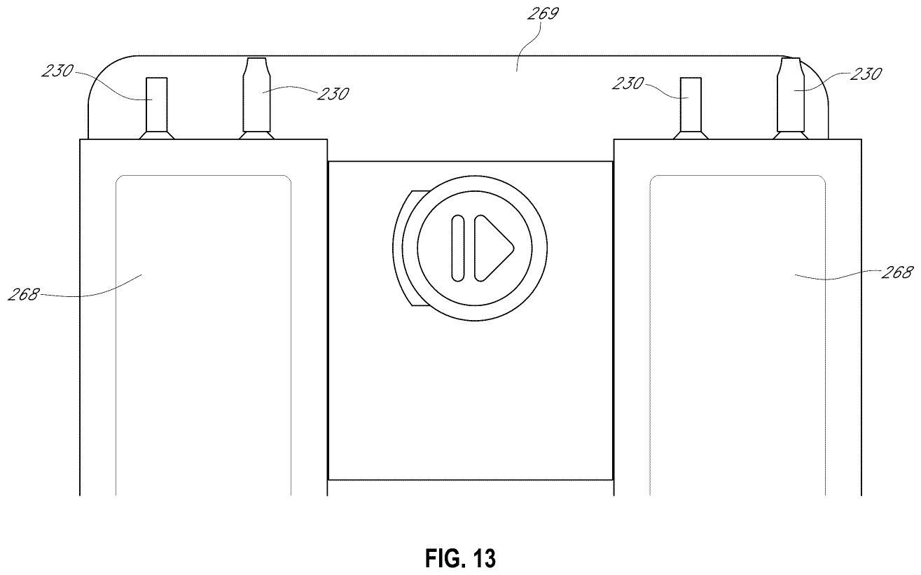

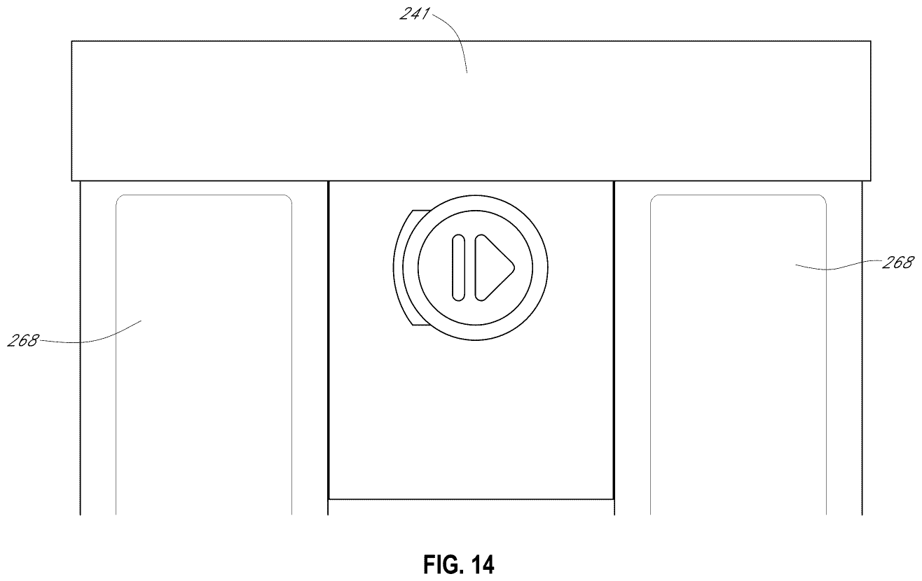

In some embodiments, a source of negative pressure (such as a pump) and some or all other components of the TNP system, such as power source(s), sensor(s), connector(s), user interface component(s) (such as button(s), switch(es), speaker(s), screen(s), etc.) and the like, can be integral with the wound dressing. The wound dressing can include various material layers described here and described in further detail in International Application No. PCT/EP2017/055225. The material layers can include a wound contact layer, one or more absorbent layers, one or more spacer layers, and a backing layer or cover layer covering the one or more absorbent and spacer layers. The wound dressing can be placed over a wound and sealed to the wound with the pump and/or other electronic components contained under the cover layer within the wound dressing. In some embodiments, the dressing can be provided as a single article with all wound dressing elements (including the pump) pre-attached and integrated into a single unit. In some embodiments, a periphery of the wound contact layer can be attached to the periphery of the cover layer enclosing all wound dressing elements as illustrated in FIG. 1A-1B. Details of the various components of the wound dressing are described in more detail in International Application No. PCT/EP2017/055225. Specifically, FIGS. 13 and 14 of International Application No. PCT/EP2017/055225 describe the dressing layers and components associated with an offset pump adjacent to an absorbent area. Such embodiments and description provide details for the materials and alternative embodiment described herein with reference to FIGS. 1A and 1B.

In some embodiments, the pump and/or other electronic components can be configured to be positioned adjacent to or next to the absorbent and/or transmission layers so that the pump and/or other electronic components are still part of a single apparatus to be applied to a patient. However, in some embodiments, the pump and/or other electronics are positioned away from the wound site. FIG. 13 of International Application No. PCT/EP2017/055225 illustrates a wound dressing with the pump and/or other electronics positioned away from the wound site. The wound dressing can include an electronics area 1361 and an absorbent area 1360. The absorbent area 1360 can include an absorbent material 1312 and can be positioned over the wound site. The electronics area 1361 can be positioned away from the wound site, such as by being located off to the side from the absorbent area 1360. The electronics area 1361 can be positioned adjacent to and in fluid communication with the absorbent area 1360. The dressing can comprise a wound contact layer (not shown), a spacer layer (not shown), an absorbent layer 1312, a moisture vapor permeable film or cover layer 1313 positioned above the contact layer, spacer layer, absorbent layer, or other layers of the dressing. The wound dressing layers of the electronics area and the absorbent layer can be covered by one continuous cover layer 1313.

FIG. 14 of International Application No. PCT/EP2017/055225 illustrates an embodiment of a wound dressing with the pump and electronic components offset from the absorbent area of the dressing positioned over the wound. The wound dressing can comprise a wound contact layer 1310 and a moisture vapor permeable film or cover layer 1313 that enclose an absorbent area 1360 and an electronics area 1361. The cover layer 1313 can seal to the wound contact layer 1310 at a perimeter. The dressing can comprise an upper spacer layer or first spacer layer 1317 above the layers of the absorbent area. The spacer material or upper spacer layer 1317 can enable an air pathway between the two areas of the dressing.

As described with reference to FIG. 14 of International Application No. PCT/EP2017/055225, the absorbent area 1360 of the dressing can comprise a second spacer layer 1311 or lower spacer layer and an absorbent layer 1322 positioned above the wound contact layer 1310. The second spacer layer 1311 can allow for an open air path over the wound site. The absorbent layer 1322 can comprise a super absorber positioned in the absorbent area 1360 of the dressing. The absorbent layer 1322 can retain wound fluid within thereby preventing fluid passage of wound exudates into the electronics area 1361 of the dressing. The wound fluids can flow through the wound contact layer 1310, to the lower spacer layer 1311, and into the absorbent layer 1322. The wound fluids are then spread throughout the absorbent layer 1322 and retained in the absorbent layer 1322 as shown by the directional arrows for wound fluids in FIG. 14 of International Application No. PCT/EP2017/055225.

As described with reference to FIG. 14 of International Application No. PCT/EP2017/055225, in some embodiments, the electronics area 1361 of the dressing can comprise a plurality of layers of spacer material 1351 and electronic components 1350 embedded within the plurality of layers of spacer material 1351. The layers of spacer material can have recesses or cut outs to embed the electronic components within whilst providing structure to prevent collapse. The electronic components 1350 can include a pump, power source, controller, and/or an electronics package. A partition 1362 can be positioned between the absorbent area 1360 and the electronics area 1361. The partition 1362 can separate the absorbent layer 1322 and lower air flow spacer layer 1311 from the electronic housing segment of the dressing in the electronic area. The partition 1362 can prevent wound fluid from entering the electronic housing section of the dressing. In some embodiments, the partition can be a non-porous dam or other structure. The non-porous dam 1362 can comprise a cyanoacrylate adhesive bead or a strip of silicone. The air pathway through the dressing is shown in FIG. 14 of International Application No. PCT/EP2017/055225 by directional arrows. The air flows through the wound contact layer 1310, the lower spacer layer 1311, and the absorbent layer 1322 and into the first spacer layer 1317. The air can travel horizontally through the first spacer layer 1317 over and around the partition 1362 into the electronics area of the dressing.

A pump exhaust 1370 can be provided to exhaust air from the pump to the outside of the dressing. The pump exhaust can be in communication with the electronics area 1361 and the outside of the dressing. In some embodiments, the pump exhaust 1370 can be a flexible fluidic connector that comprises a 3D material that allows for pressure to be applied without collapse of the exhaust port. Examples of an application where additional disclosure relating to the 3D material can be found include US Publication No. 2015/0141941, titled “Apparatuses and Methods for Negative Pressure Wound Therapy” published on May 21, 2015. The disclosure of this patent application is hereby incorporated by reference in its entirety.

As used herein the upper layer, top layer, or layer above refers to a layer furthest from the surface of the skin or wound while the dressing is in use and positioned over the wound. Accordingly, the lower surface, lower layer, bottom layer, or layer below refers to the layer that is closest to the surface of the skin or wound while the dressing is in use and positioned over the wound. Additionally, the layers can have a proximal wound-facing face referring to a side or face of the layer closest to the skin or wound and a distal face referring to a side or face of the layer furthest from the skin or wound.

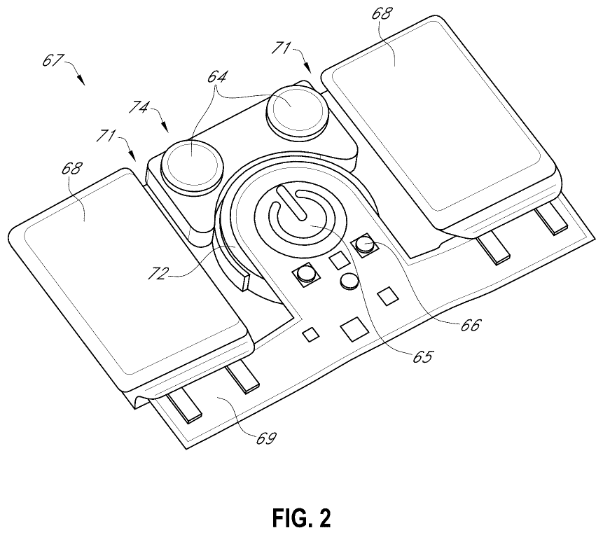

FIGS. 1A-1B illustrates a wound dressing apparatus incorporating the pump and/or other electronic components within the wound dressing and offset from the absorbent layer. FIGS. 1A-1B illustrates an embodiment of the absorbent area 60 and electronics area 61 of a wound dressing with integrated pump and/or electronics. The absorbent area can be similar to the absorbent area described with reference to FIGS. 13 and 14 of International Application No. PCT/EP2017/055225. In some embodiments, as shown in FIG. 1B, the absorbent area 60 comprises a spacer layer 52 positioned above the wound contact layer 51. An absorbent layer 50 can be provided above the spacer layer 52. In some embodiments, the electronics area 61 can include an electronics unit (not shown) and the electronics unit can be surrounded by a casing 73 (shown in FIGS. 2B-2C) forming an electronics cassette 62 or electronics cradle as described herein. In some embodiments, as described in more detail herein, the electronics unit can be provided in the electronics area without a casing. In some embodiments, the electronics cassette 62 and/or electronics unit is provided directly over the wound contact layer. In other embodiments, the electronics cassette 62 and/or electronics unit can be placed above a layer of wicking material, absorbent material, or spacer material that sits above the wound contact layer 51 of the dressing. For example, as shown in FIG. 1B, the cassette 62 and/or electronics unit may be positioned over the spacer layer 52. In some embodiments, the spacer layer 52 can be a single layer of material extending below the electronics cassette 62 and/or electronics unit and the absorbent material 50. Thus, in some embodiments, the spacer layer 52 extends continuously through the absorbent area 60 and the electronics area 61. In alternative embodiments, the spacer layer below the electronics cassette 62 and/or electronics unit can be a different spacer layer than the spacer layer below the absorbent material 50. The spacer layer 52, absorbent material 50, and electronics cassette and/or electronics unit can be covered with a cover layer 63 that seals to a perimeter of the wound contact layer 51 as shown in FIGS. 1A-1B.

The electronics area 61 can include an electronics cassette 62 and/or electronics unit positioned below the cover layer 63 of the dressing. In some embodiments, the electronics unit can be surrounded by a material to enclose or encapsulate a negative pressure source and electronics components by surrounding the electronics. In some embodiments, this material can be a casing. In some embodiments, the electronics unit can be encapsulated or surrounded by a protective coating, for example, a hydrophobic coating as described herein. The electronics unit can be in contact with the dressing layers in the absorbent area 60 and covered by the cover layer 63. As used herein, the electronics unit or cassette 62 includes a lower or wound facing surface that is closest to the wound (not shown in FIG. 1A) and an opposite, upper surface, furthest from the wound when the wound dressing is placed over a wound.

FIG. 1B illustrates a cross sectional view of the wound dressing embodiments with an off-set integrated pump and electronics. In some embodiments, the wound dressing includes a wound contact layer 51 and a spacer layer 52. Above the spacer layer 52, the wound dressing includes an electronics cassette 62 or electronics unit 62 within the electronics area 61 adjacent to or next to the absorbent material 50 within the absorbent area 60. A top film or cover layer 63 can be applied or laminated over the top surface of the electronics cassette 62 or electronics unit 62 and the absorbent layer 50. The cover layer 63 can be sealed to the wound contact layer 51 at a perimeter of the dressing.

### Electronics Unit

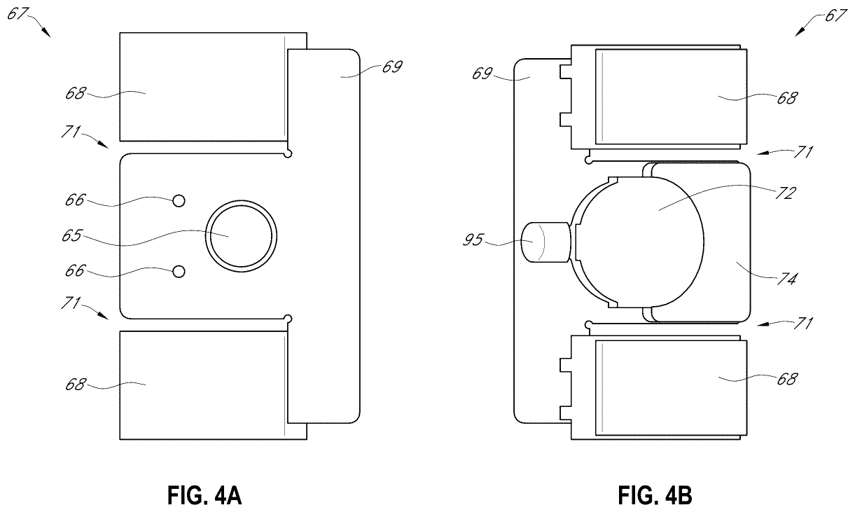

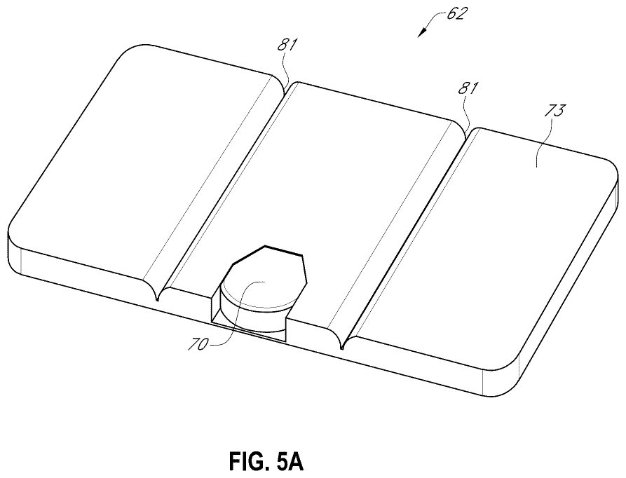

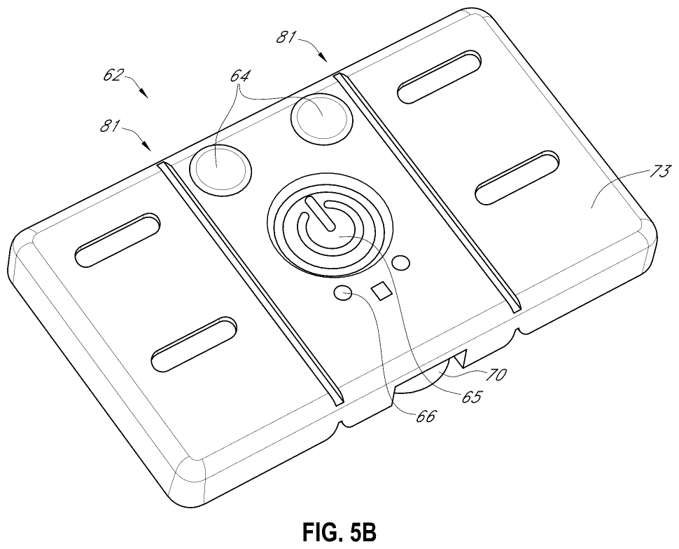

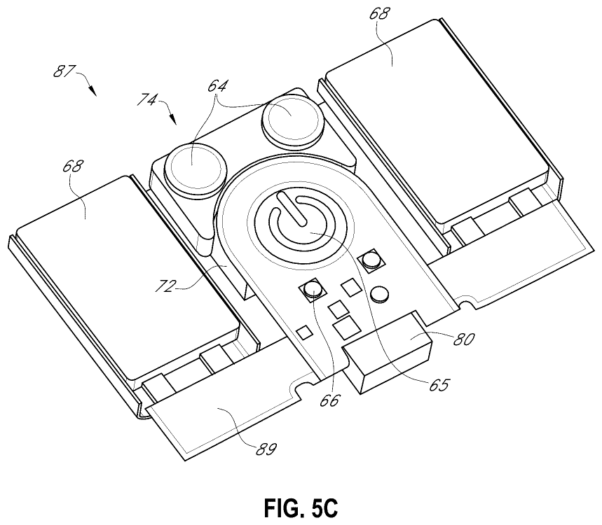

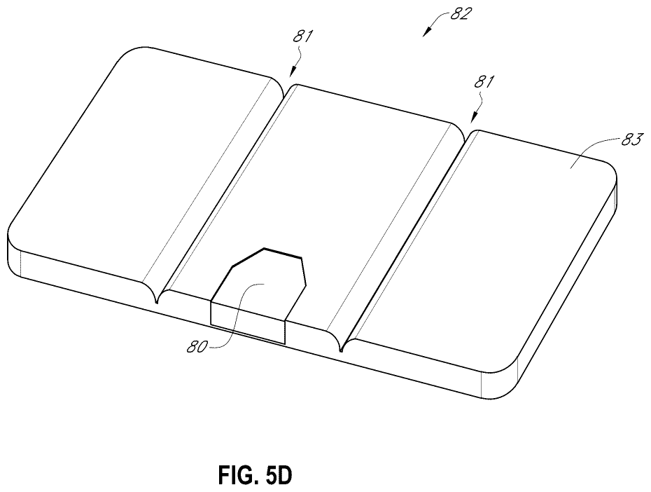

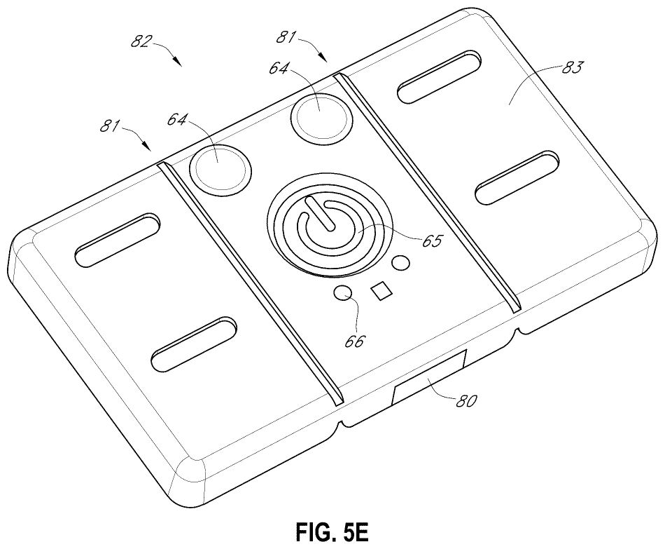

FIGS. 2, 3A-3D, and 4A-4B illustrate embodiments of an electronics unit 67 that can be incorporated into a wound dressing. The electronics unit 67 of FIGS. 2, 3A-3D, and 4A-4B illustrate multiple views of embodiments of the electronics unit 67 without an electronics casing or other dressing material. FIGS. 2, 3B, and 4A illustrate a top view of embodiments of the electronics unit 67. FIGS. 4A and 5B illustrates a bottom or wound facing surface of the electronics unit 67.

FIGS. 2, 3A-3D, and 4A-4B illustrates an embodiment of an electronics unit 67 including a pump 72 and one or more batteries 68 or other power source to power the pump 72 and other electronics. The pump can operate at about 27 volts or about 30 volts. The two batteries can allow for a more efficient voltage increase (6 volts to 30 volts) than would be possible with a single battery.

The batteries 68 can be in electrical communication with a flexible circuit board 69. In some embodiments, one or more battery connections are connected to the top surface of the flexible circuit board 69. In some embodiments, the flexible circuit board can have other electronics incorporated within. For example, the flexible circuit board may have various sensors including, but not limited to, one or more pressure sensors, temperature sensors, optic sensors and/or cameras, and/or saturation indicators. The flexible circuit board 69 illustrated in FIG. 2 communicates with a power switch 65 and the pump 72 and is folded or stepped down around the sides of a pump inlet protection (not shown) to contact the bottom surface of the battery contacts with the top surface of the flexible circuit board 69. In some embodiments, the flexible circuit board can have other electronics incorporated within. For example, the flexible circuit board may have various sensors including, but not limited to, one or more pressure sensors, temperature sensors, optic sensors and/or cameras, and/or saturation indicators.

As illustrated in FIGS. 2, 3A-3D, and 4A-4B, the electronics unit 67 can include single button 65 on the upper surface of the unit. The single button 65 can be used as an on/off button or switch to stop and start operation of the pump and/or electronic components. In some embodiments, the switch 65 can be positioned on a top surface of the flexible circuit board 64. The switch 65 can be a dome type switch configured to sit on the top of the pump 72. Because the switch is situated within the dressing the cover layer can be easily sealed around or over the wound. In some embodiments, the cover layer can have an opening or hole positioned above the switch. The cover layer can be sealed to the outer perimeter of the switch 65 to maintain negative pressure under the wound cover. The switch can be placed on any surface of the electronics unit and can be in electrical connection with the pump.

In some embodiments, the upper surface of the electronics unit 67 can include one or more indicators 66 for indicating a condition of the pump and/or level of pressure within the dressing. In some embodiments, the indicators can be provided on the flexible circuit board 69. The indicators can be small LED lights or other light source that are visible through the dressing material or through holes in the dressing material above the indicators. The indicators can be green, yellow, red, orange, or any other color. For example, there can be two lights, one green light and one orange light. The green light can indicate the device is working properly and the orange light can indicate that there is some issue with the pump (e.g. dressing leak, saturation level of the dressing, and/or low battery).

The electronics unit 67 can also include one or more vents or exhausts 64 in communication with the pump outlet. The vent or exhaust 64 can be positioned on a pump outlet or exhaust mechanism 74. The pump outlet or exhaust mechanism 74 can be positioned at the outlet of the pump and extending to the upper surface of the electronics unit. As shown in FIGS. 2, 3A-3D, and 4A-4B, the pump outlet exhaust mechanism 74 is attached to the outlet of the pump and provides communication with the top surface of the dressing. In some embodiments, the exhaust mechanism 74 can be attached to the back surface of the pump and can extend out of the pump at a 90-degree angle from the pump orientation to communicate with the top surface of the dressing. The exhaust mechanism 74 can include an antibacterial membrane and/or a non-return valve. The exhausted air from the pump can pass through the pump outlet and exhaust mechanism 74. In some embodiments, the cover layer 63 (shown in FIG. 1A-1B) can include apertures or holes positioned above the exhaust vents 64 and/or membrane. The cover layer 63 can be sealed to the outer perimeter of the exhaust 64 to maintain negative pressure under the wound cover 63. In some embodiments, the exhausted air can be exhausted through the gas permeable material or moisture vapor permeable material of the cover layer. In some embodiments, the cover layer does not need to contain apertures or holes over the exhaust and the exhausted air is expelled through the cover layer. In some embodiments, the pump outlet mechanism 74 can be a custom part formed to fit around the pump as shown in FIG. 2.

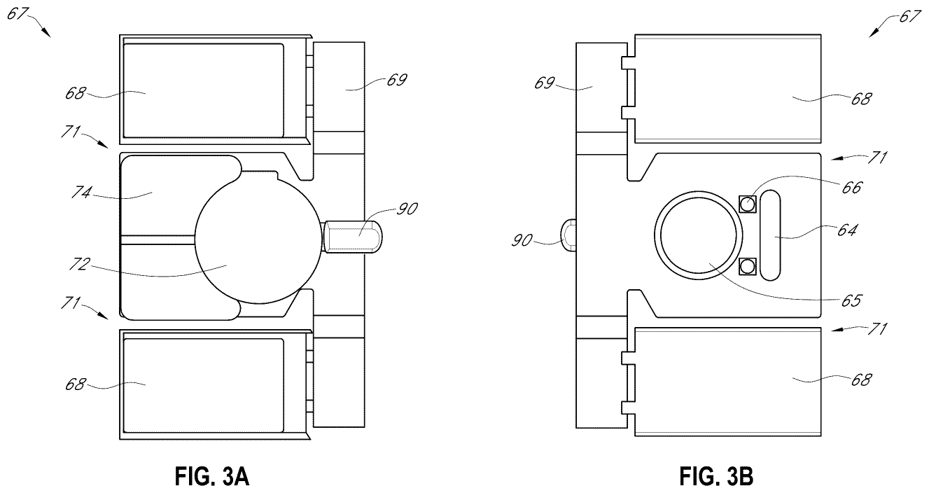

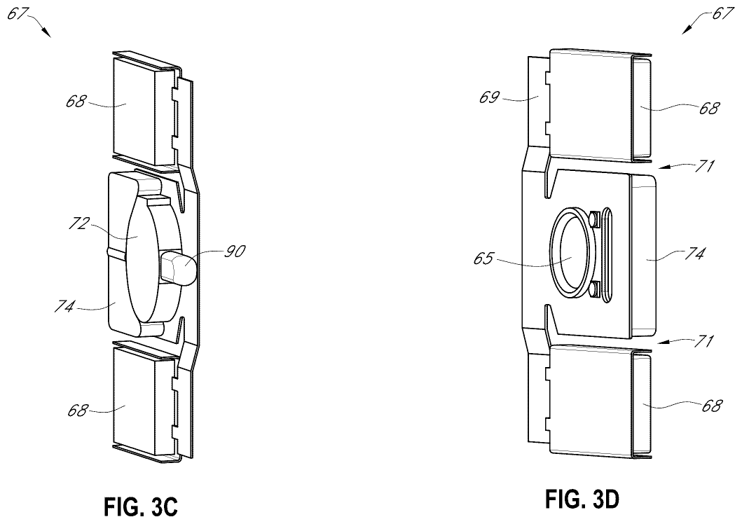

The electronic unit 67 can include a pump inlet protection mechanism (shown in FIGS. 3A-4B as 90 and 95) positioned on the portion of the electronics unit closest to the absorbent area and aligned with the inlet of the pump. The pump inlet protection mechanism can be positioned between the pump inlet and the absorbent area or absorbent layer of the dressing. The pump inlet protection mechanism can be formed of a hydrophobic material to prevent fluid from entering the pump.

The electronic unit 67 can include a pump inlet protection mechanism 90 or 95 shown in FIGS. 3A-3D and 4A-4B positioned on the portion of the electronics unit closest to the absorbent area and aligned with the inlet of the pump. The pump inlet protection mechanism 90 or 95 is positioned between the pump inlet and the absorbent area or absorbent layer of the dressing. The pump inlet protection mechanism 90 or 95 can be formed of a hydrophobic material to prevent fluid from entering the pump. In some embodiments, the pump inlet protection mechanism can be positioned on the pump inlet. In some embodiments, the pump inlet protection mechanism can be formed to fit around or over the pump inlet. The pump inlet protection mechanism 90 or 95 as shown in FIGS. 3A-3D and 4A-4B can be dome shaped, rectangular, curved, or can be any other shape to provide support to the flexible circuit board 69 and protect the pump inlet from fluid within the dressing. The pump inlet protection mechanism 90 or 95 can be a preformed sintered piece of material custom designed to fit the pump outlet and/or shape the flexible circuit board. In some embodiments, the pump 72, pump inlet protection mechanism 90 or 95, and pump outlet or exhaust mechanism 74 can be combined to formed a pump assembly as used herein.

In some embodiments, the components of the electronics unit 67 may include a protective coating to protect the electronics from the fluid within the dressing. The coating can provide a means of fluid separation between the electronics unit 67 and the absorbent materials of the dressing. The coating can be a hydrophobic coating including, but not limited to, a silicone coating or polyurethane coating. The pump inlet component (90 and 95 shown in FIGS. 3A-3D and 4A-4B) can be used to protect the pump from fluid on the inlet and the pump outlet mechanism 74 can include a non-return valve that protects fluid from entering the outlet as described herein.

The electronics unit 67 includes one or more slits, grooves or recesses 71 in the unit between the pump and the two batteries. The slits, grooves or recesses 71 can allow for the electronics unit 67 to be flexible and conform to the shape of the wound. The unit 67 can have two parallel slits, grooves or recesses 71 forming three segments of the electronics unit 67. The slits, grooves or recesses 71 of the unit 67 create hinge points or gaps that allows for flexibility of the electronics unit at that hinge point. The pump exhaust 64, switch 65, and indicators 66 are shown on the top surface surrounded by the electronics unit 67. As illustrated, one embodiment of the electronics unit 67 has two hinge points to separate the unit into three regions or panels, for example one to contain one battery, one to contain the pump and one to contain another battery. In some embodiments, the slits, grooves or recesses may extend parallel with a longitudinal axis of the dressing.

### Electronics Cassette

FIGS. 5A-5E illustrate embodiments of components of the electronics cassette 62. FIGS. 5A-5B illustrates an embodiment of an electronic cassette 62 with a rounded pump inlet 70. FIG. 5A illustrates a bottom view of an embodiment of the electronics cassette 62 showing the wound facing side of the electronics cassette 62. FIG. 5B illustrates a top view of an embodiment of the electronics cassette 62 showing the upper surface of the cassette.

FIGS. 5A-5B illustrate the electronics cassette 62 including a casing 73 as shown in FIGS. 5A-5B surrounding the electronics unit 67 described with reference to FIG. 2. In some embodiments, the casing 73 can be used to support and encapsulate the electronics unit 67. In some embodiments, the casing can be made from a plastic material. In some embodiments, the casing 73 may be formed of a foam, an absorbent material, and/or a wicking material and may be in fluid communication with the absorbent area of the dressing. The encapsulation with a foam, an absorbent material, and/or a wicking material can protect against the pooling of the liquid around the electronics area. The casing 73 can be covered on the top surface by a top film or cover layer when incorporated into a dressing. The top film or cover layer can cover the top surface of casing 73 as well as the layers of the absorbent area. The cover layer can have a hole for the exhaust and/or the switch area as described herein. The encapsulation of the electronics unit 67 with the casing 73 can provide support for the flexible printed circuit board (PCB) 69 and other components of the electronics unit 67. The casing 73 can provide support to the electronics unit 67, remain comfortable for the patient, and offer a means of continuous surface and height profile for the electronics unit.

FIG. 5C illustrates an embodiment of a pump and electronics unit 87 without showing an electronics casing 83 surrounding the electronics unit thereby exposing the electronics unit 67 with the various electronics. FIGS. 5D-5E illustrated the electronics unit 87 enclosed in an electronic casing 83 forming the electronic cassette 87.

FIG. 5C illustrates an embodiment of an electronics unit 87 with a rectangular pump inlet protection mechanism 80. FIGS. 5D-5E illustrate an embodiment of an electronic cassette 82 with a casing 83 enclosing the electronic unit 87 shown in FIG. 5C. FIG. 5D illustrates the bottom surface or wound facing surface of the electronics cassette 82. FIG. 5E illustrates the top surface of the electronics cassette 82.

As illustrated in FIGS. 5B-5C and 5E, the electronics cassette 62 or 82 can allow the button 65 to be visible on the upper or top surface of the cassette. In some embodiments, the electronics casing 73 or 83 can have a hole aligned over the button 65 of the electronics unit. In other embodiments, the electronics casing 73 or 83 can be positioned over at least a portion of the switch 65 of the electronics unit but can still allow for the switch to be depressed and/or activated through the material. The switch or button 65 can be placed on any surface of the electronics cassette as long as it is in an electrical connection with the pump. In some embodiments, the switch or button 65 can be placed over the battery. In some embodiments, the battery profile is not as high as the pump and can provide room to place the switch over the battery.

The one or more vents 64 are shown on the top surface of the electronics cassette 62 or 82 as illustrated in FIGS. 5B-5C and 5E. In some embodiments, the electronics casing 73 or 83 can have holes where the vents 64 of the electronics unit align with. In other embodiments, the electronics casing 73 or 83 can have a gas permeable material positioned over at least a portion of the vents 64 of the electronics unit.

In some embodiments, the one or more indicators 66 can be visible on the upper surface of the electronics cassette 62 or 82 for indicating a condition of the pump and/or level of pressure within the dressing. In some embodiments, the electronics casing 73 or 83 can have holes that align with the indicators 66 of the electronics unit. In other embodiments, the electronics casing 73 or 83 can have a transparent material positioned over at least a portion of the indicators 66 of the electronics unit.

The batteries 68 can be in electrical communication with a flexible circuit board 69. In some embodiments, the flexible circuit board can have other electronics incorporated within. For example, the flexible circuit board may have various sensors including, but not limited to, one or more pressure sensors, temperature sensors, optic sensors and/or cameras, and/or saturation indicators.

In such embodiments, the components of the electronics unit 67 or 87 may include a protective coating as described herein to protect the electronics from the fluid within the dressing. The coating can be provided between the electronics and the casing 73 and 83.

The electronic cassette 62 or 82 can have an opening or aperture for the pump inlet protection mechanism 70 or 80 positioned on the inlet of the pump 72 of the electronics unit 67 or 87 and the portion of the electronics cassette closest to the absorbent area. The pump inlet protection mechanism 70, 80, 90, or 95 as shown in FIGS. 2, 3A-3D, 4A-4B, and 5A-5E can be dome shaped, rectangular, curved, or can be any other shape to provide support to the flexible circuit board 69, 89 and protect the pump inlet from fluid within the dressing. The pump inlet protection mechanism can be a preformed sintered piece of material custom designed to fit the pump outlet and/or shape the flexible circuit board.

As illustrated in FIGS. 5A and 5D, the electronics cassette 62 includes one or more slits, grooves or recesses 81 in the casing 73 or 83. The slits, grooves or recesses 81 can allow for the electronics cassette 62 to be flexible and conform to the shape of the wound. The slits, grooves or recesses 81 of the electronics cassette can align with the slits, grooves or recesses 71 of the electronics unit 67 or 87. The casing can have two parallel slits, grooves or recesses 81 forming three segments in the electronics cassette 62. The slits, grooves or recesses 81 of the casing 73 create hinge points or gaps that allows for flexibility of the casing at that hinge point.

FIGS. 5B and 5E illustrates embodiments of a top view of the electronics cassette 62 or 82. The pump exhaust 64, switch 65, and indicators 66 are shown on the top surface surrounded by the casing 73 or 83. As illustrated, one embodiment of the electronics cassette 62 or 82 has two hinge points, slits, grooves or recesses 81 on the top or upper surface of the cassette to separate the cassette into three regions or panels, for example one to contain one battery, one to contain the pump and one to contain another battery. In some embodiments, the hinge points, slits, grooves or recesses can be on the top surface, bottom surface, or on both the top and bottom surfaces of the cassette. In some embodiments, the slits, grooves or recesses may extend parallel with a longitudinal axis of the dressing.

The electronics unit 87 and electronics cassettes for FIGS. 5C-5E are similar to the electronics unit and cassette described with reference to FIG. 5A-5B. However, the electronics unit 67 and pump inlet protection mechanism 70 of FIG. 5A-5B is similar to the electronics unit 67 and pump inlet protection mechanism 70 described with reference to FIG. 2. FIGS. 5D-5E illustrate an embodiment of the electronic cassette 82 with the electronics unit 87 and pump inlet protection mechanism 80 of FIG. 5C. FIG. 5C illustrates an embodiment of a pump and electronics unit 87 without showing the electronics casing 73 surrounding the electronics unit. The flexible circuit board 89 and pump inlet protection 80 have a different configuration than the electronic unit 67 shown in FIG. 2. In some embodiments, the portions of the flexible circuit board 89 that attaches to the batteries 68 is folded under the portion of the flexible circuit that contains the on/off switch 65. Since the flexible circuit board is folded under itself, the surface of the flexible circuit board that connects the batteries contacts can be facing the wound surface as shown in FIG. 5C. Due to this configuration, the battery contacts are not visible from the top view of the electronics unit as shown in FIG. 5C. FIGS. 5C-5E illustrate the pump inlet protection device 80 that fits to the opening in the electronics cassette.

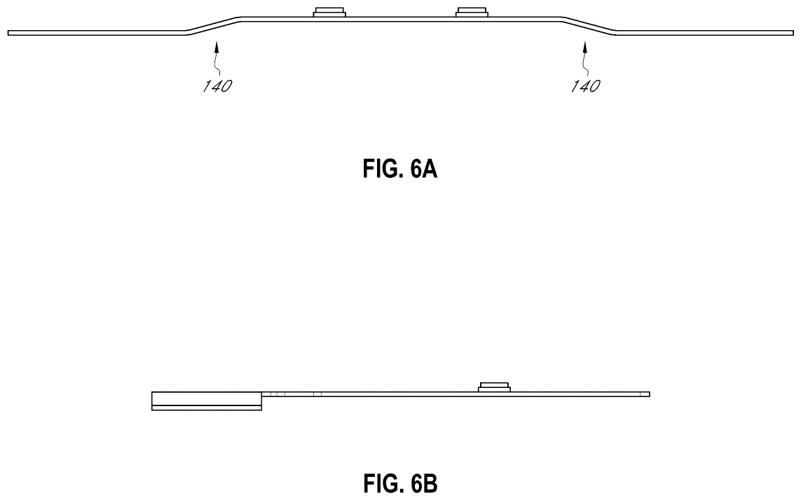

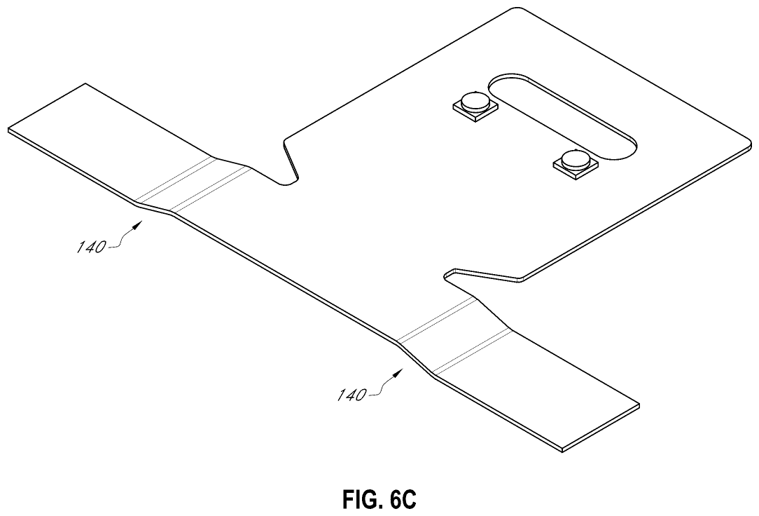

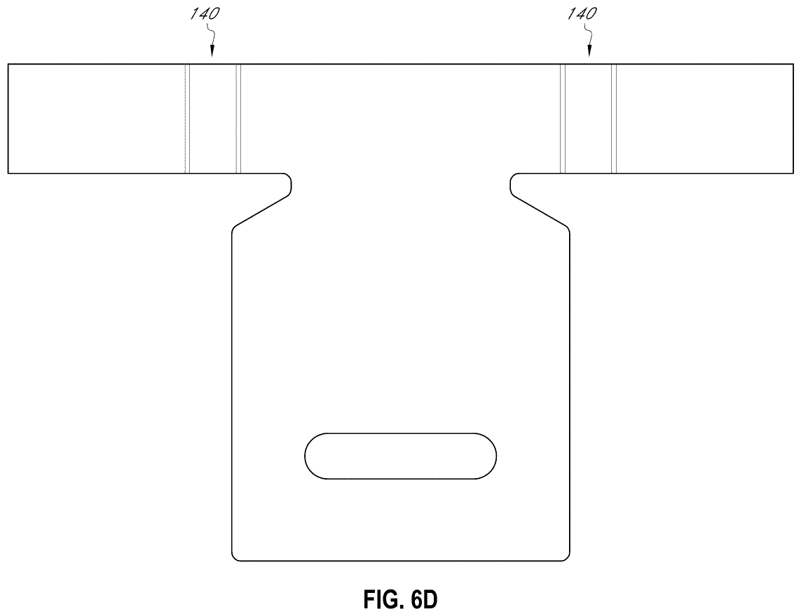

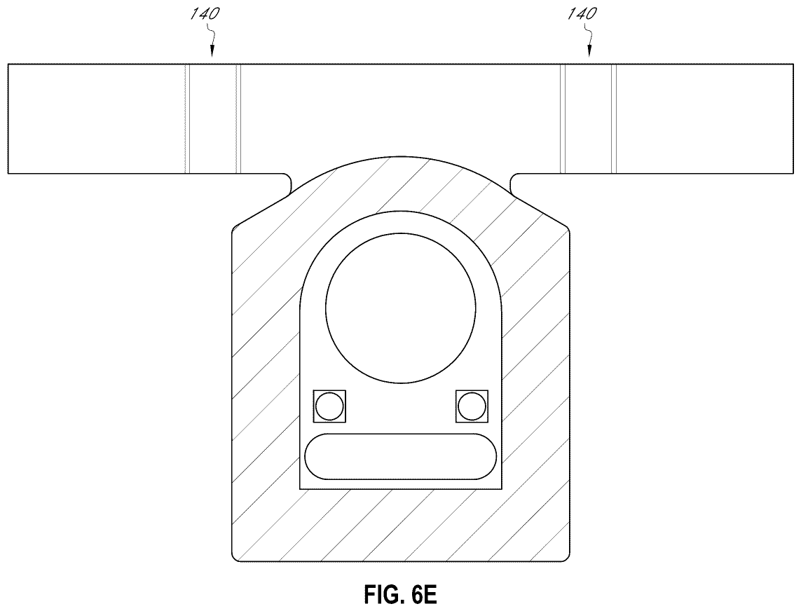

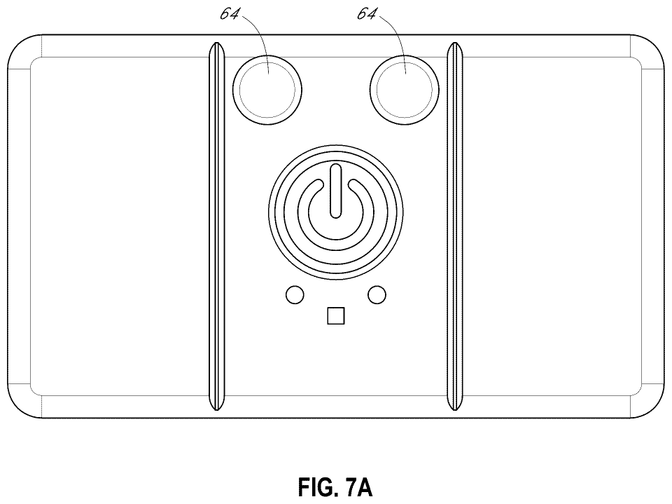

FIGS. 6A-6D illustrate an embodiment of the flexible circuit board for use in the electronics cassette. The flexible circuit board illustrated in FIGS. 6A-6D is similar to the flexible circuit board 69 described with reference to FIG. 2. The flexible circuit board includes a fold 140 to allow the flexible circuit board to be positioned under battery contacts when integrated into the electronics unit as illustrated in FIG. 2. FIG. 6E illustrates the area (hatched) that could be bonded to for sealing between the top film or electronics casing and the flexible printed circuit board.

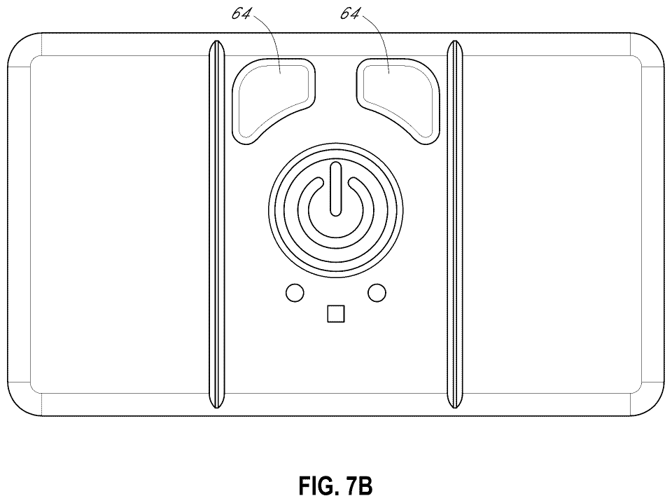

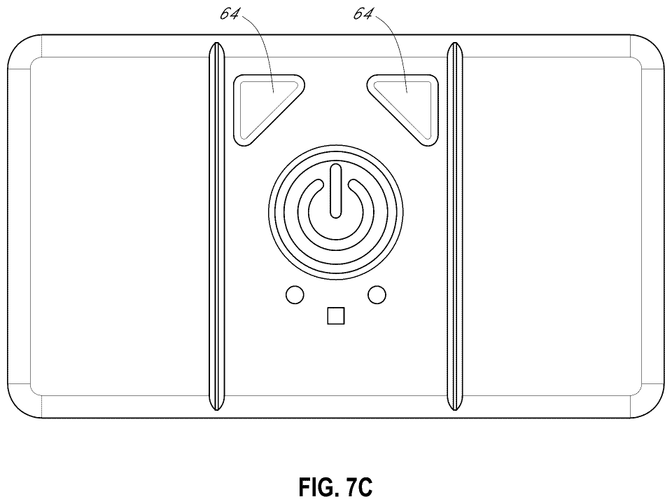

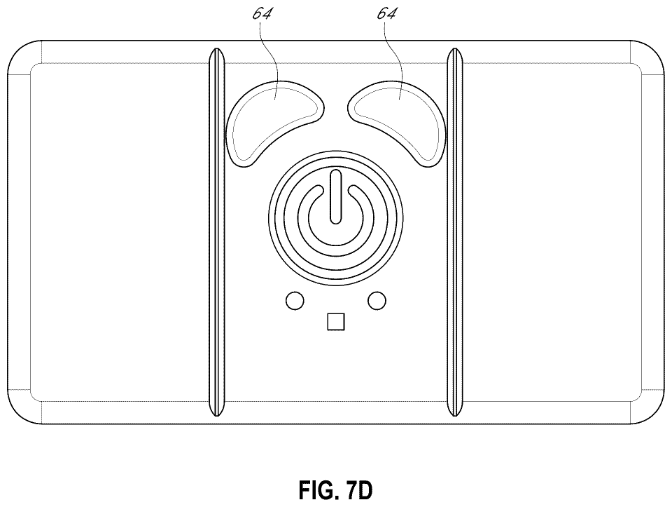

FIGS. 7A-7D illustrate embodiments of electronic cassettes with two pump exhaust vents 64 in various configurations and shapes. In some embodiments, the two pump exhaust vents 64 can be various shapes. The two pump exhaust vents 64 can be circular as shown in FIG. 7A, irregular shaped as shown in FIG. 7B, triangular as shown in FIG. 7C, crescent shaped as shown in FIG. 7D, or any other shape including but not limited to a rectangle, square, rounded rectangle, rounded square, oval, or racetrack oval shaped. The two pump exhausts allow for one exhaust to become blocked, occluded, or inoperable while still allowing the other exhaust to function.

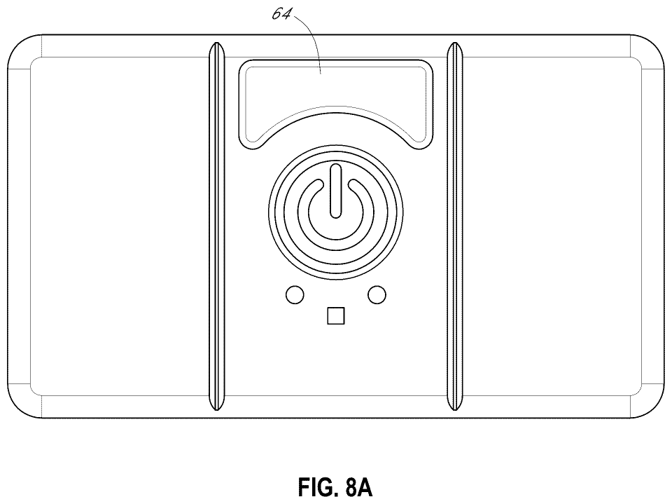

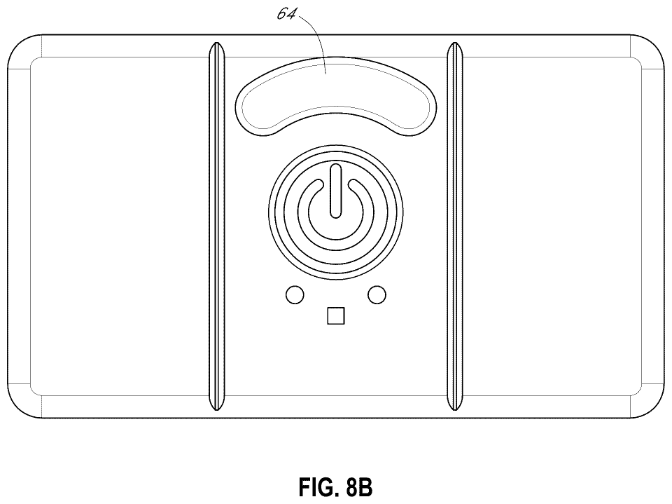

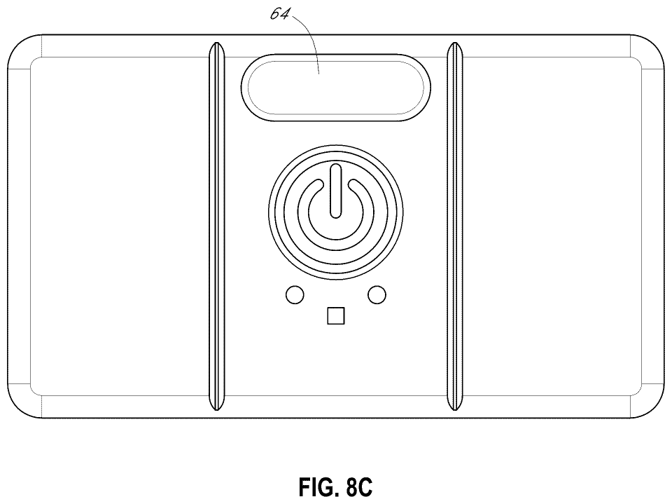

FIGS. 8A-8C illustrates embodiments of electronic cassettes with one pump exhaust vent 64 in various shapes. The pump exhaust vents 64 can be irregular shaped as shown in FIG. 8A, crescent shaped as shown in FIG. 8B, racetrack oval shaped as shown in FIG. 8C, or any other shape including, but not limited to, a circle, rectangle, square, rounded rectangle, rounded square, oval, or triangle shaped.

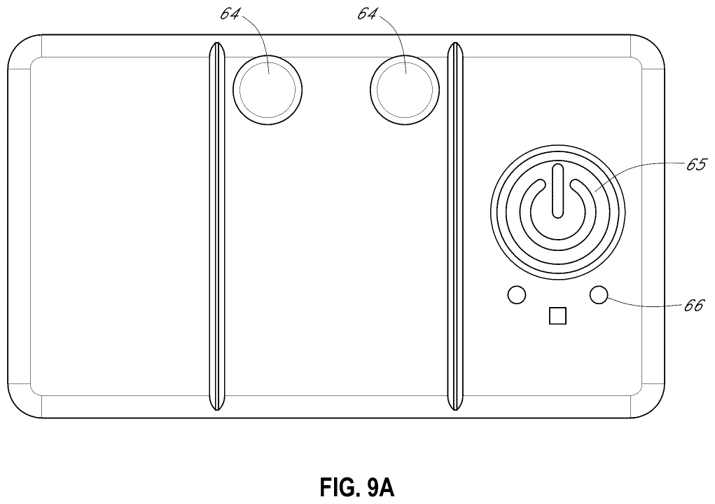

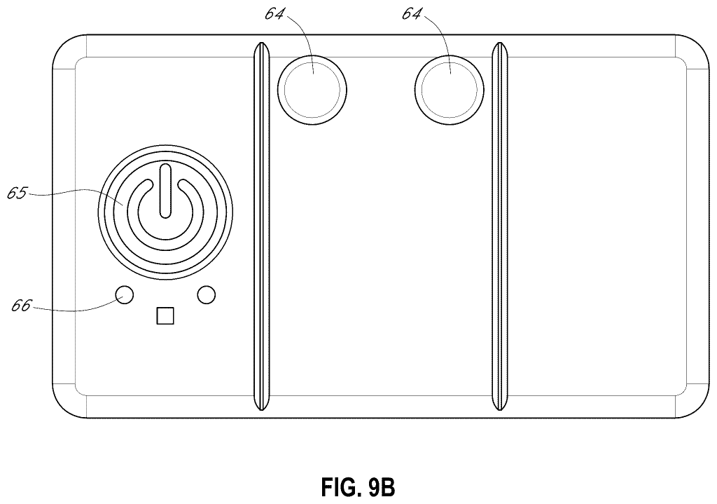

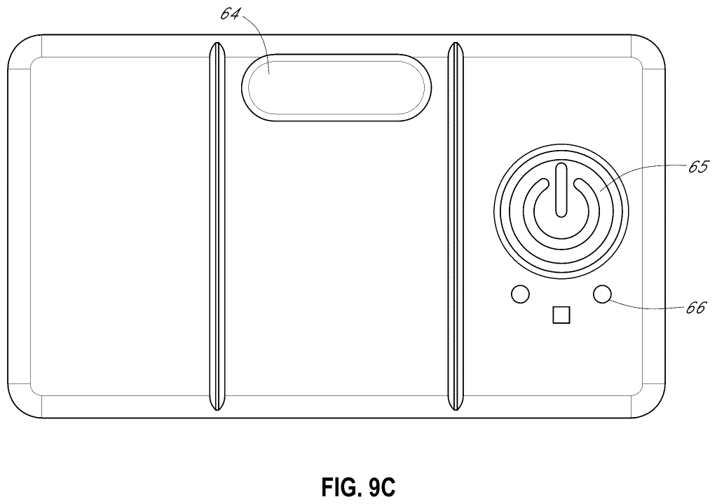

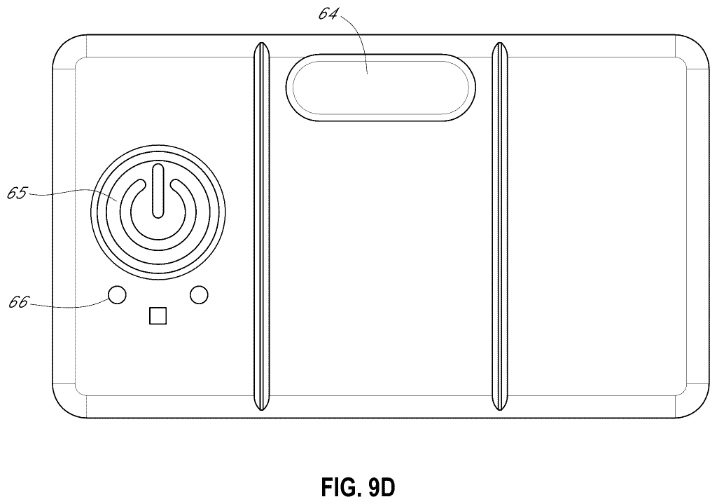

FIGS. 9A-9D illustrate various embodiments of the electronic cassette with various configurations for components on the electronic cassettes. In some embodiments, one or more pump exhaust vents 64 can be used as described with reference to FIGS. 7A-7D and 8A-8C. FIGS. 9A-9B illustrates two exhaust vents 64. FIGS. 9C and 9D illustrate an electronics cassette with one exhaust vent 64. In some embodiments, the on/off switch or button 65 and one or more indicators 66 can be positioned on different portions of the electronic cassette. In some embodiments, the on/off switch or button 65 and one or more indicators 66 can be positioned on the right panel of the electronic cassette as shown in FIGS. 9A and 9C. In some embodiments, the on/off switch or button 65 and one or more indicators 66 can be positioned on the left panel of the electronic cassette as shown in FIGS. 9B and 9D.

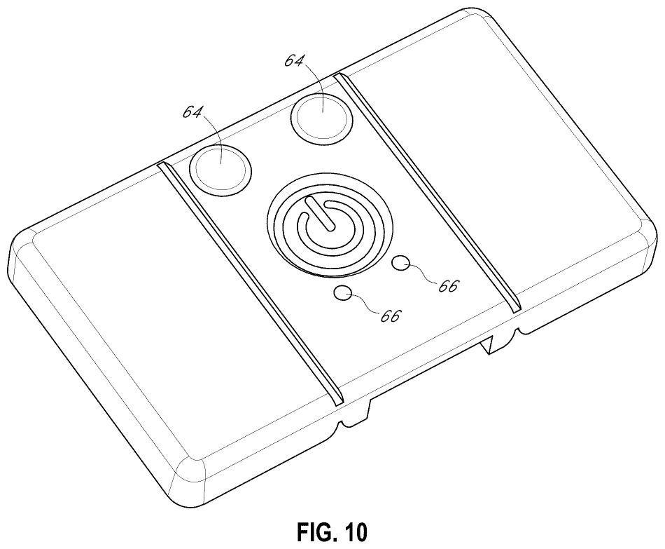

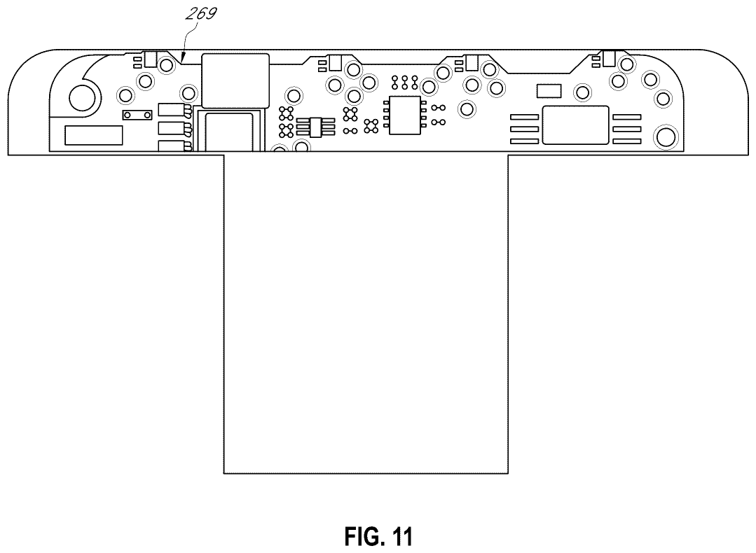

FIG. 10 illustrates an embodiment of the electronics cassette similar to the electronics cassette described with reference to FIGS. 5A-5B and 5D-5E. The electronics cassette is shown with two openings in the casing for the indicators 66 on the top surface of the cassette. The electronics cassette as illustrated in FIG. 10 includes a casing that completely covers the battery portion underlying the casing.

In some embodiments, one or more of the exhaust vents 64, switch or button 65, and one or more indicators 66 described with reference to FIGS. 7A-7D, 8A-8C, 9A-9D, and 10 can be the exhaust vents 64, switch or button 65, and/or one or more indicators 66 of the electronics unit described with reference to FIGS. 2, 3A-3D, and 4A-4B visible through holes or apertures in the casing of the electronics cassette. In some embodiments, one or more of the exhaust vents 64, switch or button 65, and/or one or more indicators 66 described with reference to FIGS. 7A-7D, 8A-8C, 9A-9D, and 10 can include material of the casing of the electronics cassette that covers the exhaust vents 64, switch or button 65, and one or more indicators 66 of the electronics unit described with reference to FIGS. 2, 3A-3D, and 4A-4B. In some embodiments, a gas permeable material can be provided as part of the casing of the electronics cassette that covers at least a portion of the exhaust vents 64 described with reference to FIGS. 7A-7D, 8A-8C, 9A-9D. In some embodiments, a transparent material can be provided as part of the casing of the electronics cassette that covers at least a portion of the one or more indicators 66 described with reference to FIGS. 7A-7D, 8A-8C, 9A-9D. In some embodiments, the material of the casing of the cassette covering at least a portion of the switch or button 65 described with reference to FIGS. 7A-7D, 8A-8C, 9A-9D can be flexible or otherwise allow for the switch or button 65 to be depressed or activated.

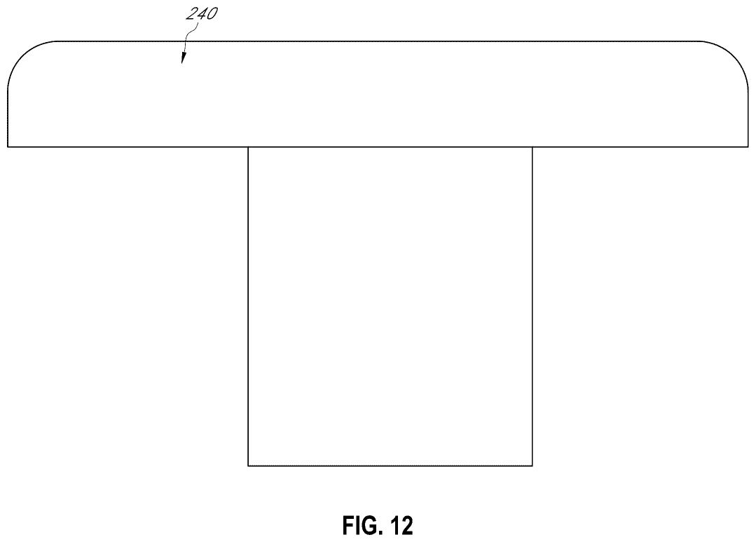

FIGS. 11-16 illustrate an assembly process for the electronics unit of the electronics cassette. FIG. 11 illustrates an embodiment of a flexible printed circuit 269. The flexible printed circuit board 269 can have all components on one side as shown in FIG. 11. In other embodiments, the flexible printed circuit board 269 can have some components on each side of the flexible printed circuit board 269. FIG. 12 illustrates the use of a polyimide tape or other material 240 that can be applied to the underside of the flexible circuit board 269 covering the components of the flexible circuit board 269. The polyimide tape or other insulating or encapsulating material can serve the purpose of encapsulation, electrical isolation, and mechanical strain relief for the batteries. In some embodiments, the electronics can be facing down to help with Electrostatic Discharge (ESD) and/or Electromagnetic Compatibility (EMC) of the device when worn.

In some embodiments, the electronics unit or assembly can be fit into a jig. The flexible circuit 269 can be fit in a jig. The batteries 268 can be fit into a jig as shown with reference to FIG. 13. In some embodiments, the batteries can include battery contacts 230 that can be spot welded to the flexible circuit board 269. A top layer of polyimide tape or other insulating or encapsulating material 241 can be applied over the assembled battery contacts as shown in FIG. 14. This material can serve the purpose of encapsulation, electrical isolation, and mechanical strain relief for the batteries.

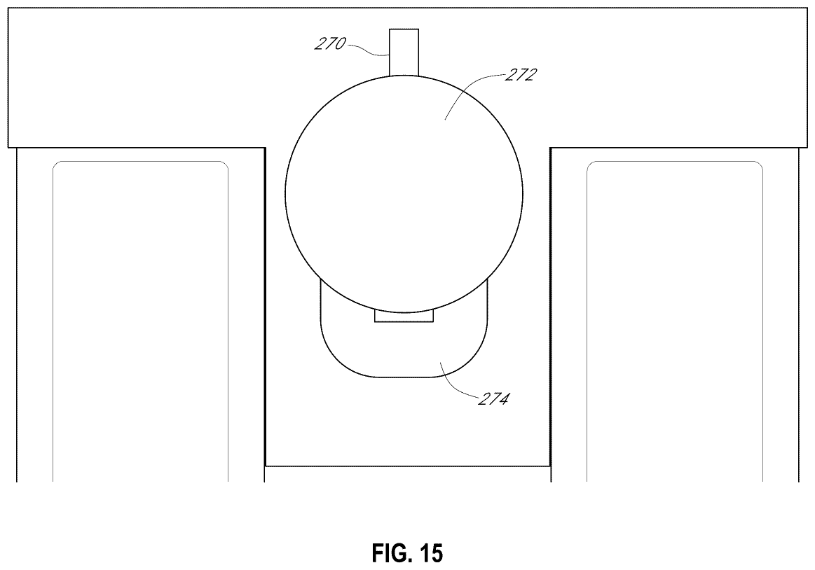

In some embodiments, a pump 272 can be pre-assembled with the custom inlet mechanism 270 and outlet mechanism 274 to form a pump assembly. As illustrated in FIG. 15, the pump assembly can be jigged into position. The pump 272 can have solder connections, so the pump assembly can be soldered onto the flexible printed circuit. The pump 272 can be encapsulated with a sealant that can be applied over these solder joints.

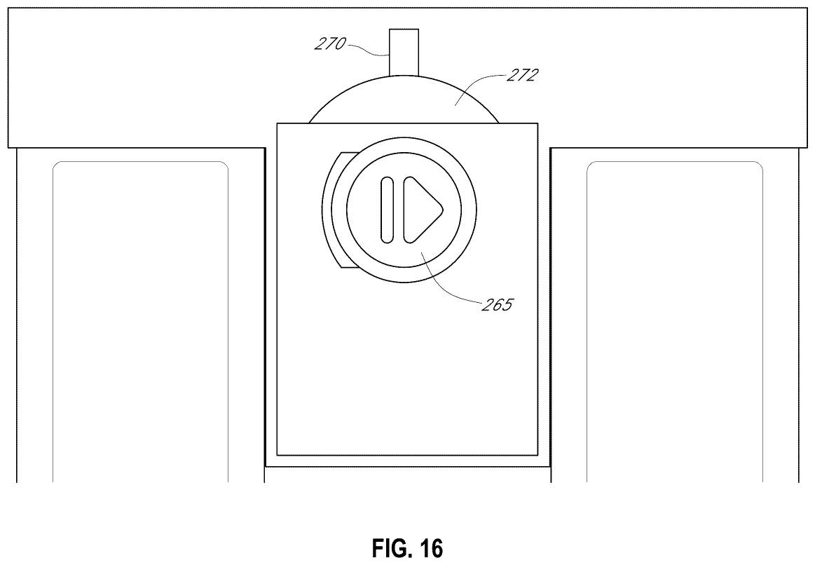

In some embodiments, the switch area 265 can be positioned on the top side of the flexible PCB 269 and over the top surface of the pump as shown in FIG. 16. In some embodiments, the portion of the PCB with the switch area 265 can be folded over onto the pump or pump assembly. In some embodiments, a spacer material can be placed over the assembly to fill the gaps between the components of the electronics and casing of the cassette.

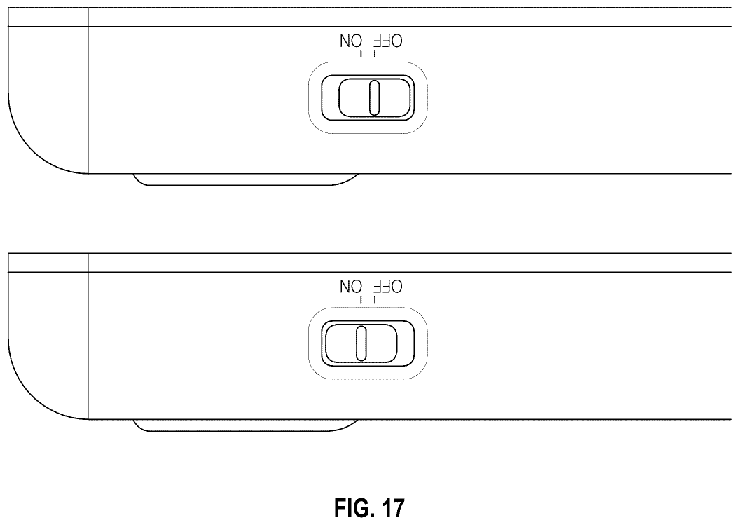

FIG. 17 illustrates an embodiment of on/off switch that can be used with a wound dressing with integrated electronics and with electronic units and cassettes described herein.

The dressings described herein incorporate electronic components in a portion of the dressing offset from the portion of the dressing placed over the wound. Components can be incorporated into the dressing to provide a barrier that stops liquid from entering into the area near the electronics or encapsulates the electronics to protect the electronic components from fluid within the dressing. In some embodiments, the electronics can be encapsulated in a hydrophobic coating to protect the electronics from fluid within the dressing. A casing can be wrapped around the encapsulated electronics unit forming the electronics cassette. The casing can be a soft or patient compatible material to minimize the discomfort of a patient when the wound dressing with integrated electronics are in contact with the wound or skin. In some embodiments, the casing can include an absorbent or wicking material that allows the flow of fluid through the casing.

### Electronics Cradle

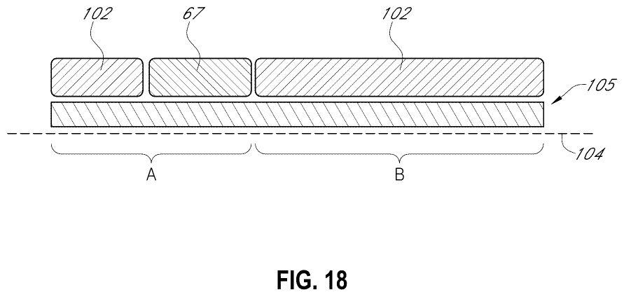

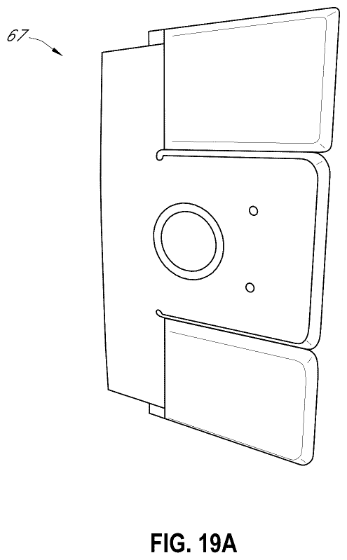

FIG. 18 illustrates an embodiment of a wound dressing incorporating an electronics unit 67 within the dressing. In some embodiments, the wound dressing can include a wound contact layer 104. The dressing can also include a spacer layer 105 which may be made of a 3D material above the wound contact layer. In some embodiments, the electronics sub assembly or electronics unit 67 can be embedded in an absorbent pad 102 towards one end of the dressing, as depicted in FIG. 18. FIG. 18 illustrates dressing layers with an electronics area A and an absorbent area B. In some embodiments, the electronics area A can include the electronics unit 67. In some embodiments, the absorbent area B can include the absorbent material 102 and can be positioned over the wound area to absorb fluid from the wound within the absorbent material or pad 102. The electronics unit 67 can be positioned in a hole in the absorbent pad 102 and can be surrounded by the absorbent pad as illustrated in FIG. 18 with the absorbent pad 102 on each side of the electronics unit 67. However, the presence of the electronics unit, since it is entirely non-absorbent, could physically inhibit the migration of wound exudate through the absorbent pad from area A to area B, the main body of the pad, and would thus result in pooling of wound exudate in area A. This can be deleterious to the patient, resulting in maceration of the skin and other potentially harmful effects.

Therefore, it can be useful to devise a dressing to facilitate normal distribution of exudate by which the fluid can be distributed through the entire pad, preventing pooling.

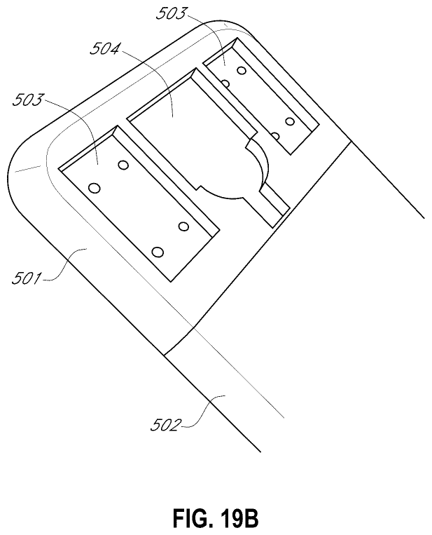

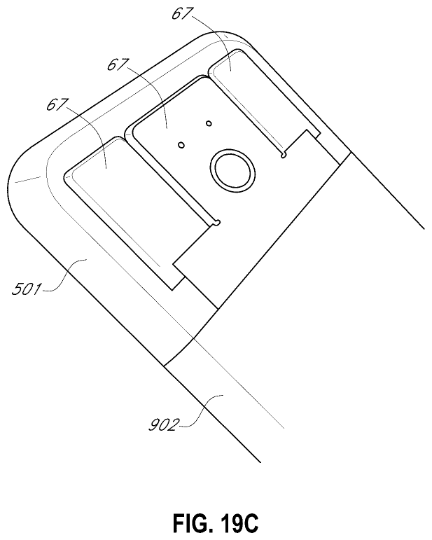

In some embodiments, a construct can be used that acts like a support mechanism or cradle for the electronics sub assembly. FIG. 19A illustrates an embodiment of the electronics unit 67. The electronics unit 67 can be coated and/or include a pump inlet and outlet protection mechanism to protect the pump from the fluid environment of the dressing as described herein. FIG. 19B illustrates an embodiment of the material of the cradle 501 that can be within, adjacent to, and/or above the absorbent dressing material 502. In some embodiments, the cradle 501 is positioned adjacent to the dressing material 502 as illustrated in FIGS. 19B and 19C. FIG. 19C illustrates an embodiment of the electronics unit 67 incorporated into the cradle 501 and adjacent to an absorbent pad 502. The shape and form of the cradle can be advantageous to production of an ergonomic form for the device. An embodiment of an electronics cradle incorporated within a wound dressing is shown in FIGS. 19B-19C. An embodiment of the electronics assembly to be used with the cradle 501 is shown in FIG. 19A.

The cradle can be constructed from hydrophilic materials (e.g. PU foam, cellulosic fiber). In some embodiments, the cradle 501 can be in intimate contact with the absorbent pad 502 to facilitate fluid distribution, thus preventing pooling, as depicted in FIGS. 19B-19C. In some embodiments, the electronics cradle 501 can be in direct contact with the wound contact layer (not shown).

The cradle can include recesses, cutouts, or slots 503 and 504 within the cradle material 501 for receiving components of the electronics unit 67 as illustrated in FIG. 19B. The electronics unit 67 can be fit into the recesses 503 and 504 of the cradle material 501. In some embodiments, the cradle can include one or more recess 503 shaped to receive the batteries and a recess 504 shaped to receive the pump with the pump inlet and pump outlet mechanisms of the electronics unit as illustrated in FIG. 19B. In some embodiments, once the electronics unit is fit within the cradle, the top of the electronics unit can be flush with or can have a similar height profile as the electronics cradle material. The cradle can be a soft or patient compatible material to minimize the discomfort of a patient when the wound dressing with integrated electronics are in contact with the wound or skin. In some embodiments, the cradle can include an absorbent or wicking material that allows the flow of fluid through the casing.

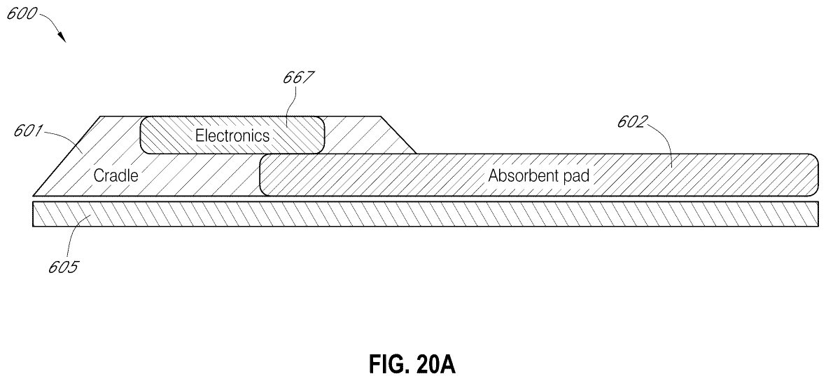

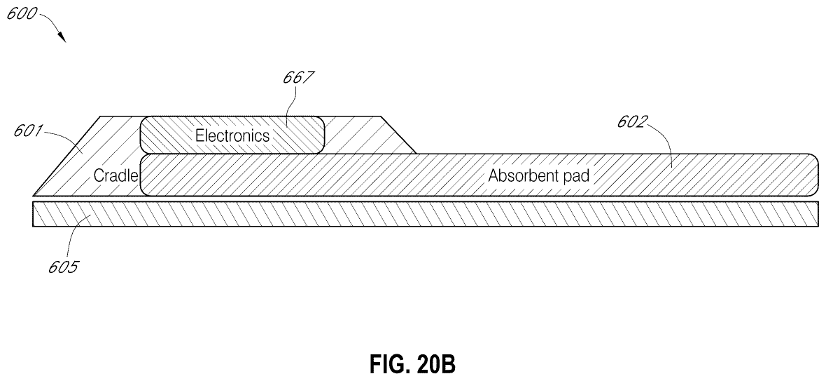

FIGS. 20A-20B illustrate an embodiment of a wound dressing 600 incorporating the electronics unit 667 and electronics cradle 601 adjacent to and at least partially overlapping the absorbent pad 602. In such embodiment, the cradle 601 material is formed to house the electronics unit 667 and a portion of the absorbent pad 602. In some embodiments, this configuration can provide intimate contact between absorbent pad 602 and the cradle 601. In some embodiments, the cradle 601 and absorbent pad 602 can be positioned over a transmission or spacer layer 605. As illustrated in FIGS. 20A-20B, the relative positions of the components can be varied as necessary. In some embodiments, the cradle 601 may be formed of the same material as the absorbent pad 602. In other embodiments, the cradle 601 may also be formed of a dissimilar material than the absorbent pad 602, e.g. absorbent polyurethane foam. In some embodiments, the cradle 601 may be formed using vacuum forming or thermoforming processes.

In some embodiments, the absorbent components and electronics components can be overlapping but offset. For example, a portion of the electronics area can overlap the absorbent area, for example overlapping the superabsorber layer, but the electronics area is not completely over the absorbent area. Therefore, a portion of the electronics area can be offset from the absorbent area and only provided over the cushioning transmission or spacer layers 605. The dressing layer and electronic components can be enclosed in a wound contact layer (not shown) positioned below the lower most layer and a cover layer (not shown) positioned above the absorbent layer and electronics. The wound contact layer and cover layer can be sealed at a perimeter enclosing the dressing components. In some embodiments, the cover layer can be in direct physical contact with the absorbent material, the cradle and/or electronics unit. In some embodiments, the cover layer can be sealed to a portion of the electronics unit and/or cassette, for example, in areas where holes or apertures are used accommodate the electronic components (e.g. a switch and/or exhaust) as described herein with reference to the electronics cassette.

Dressing Assembly with Integrated Electronics

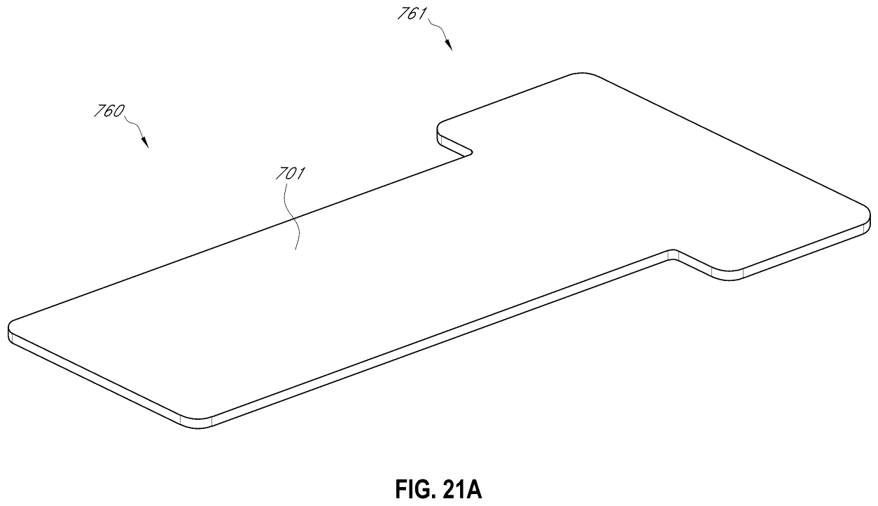

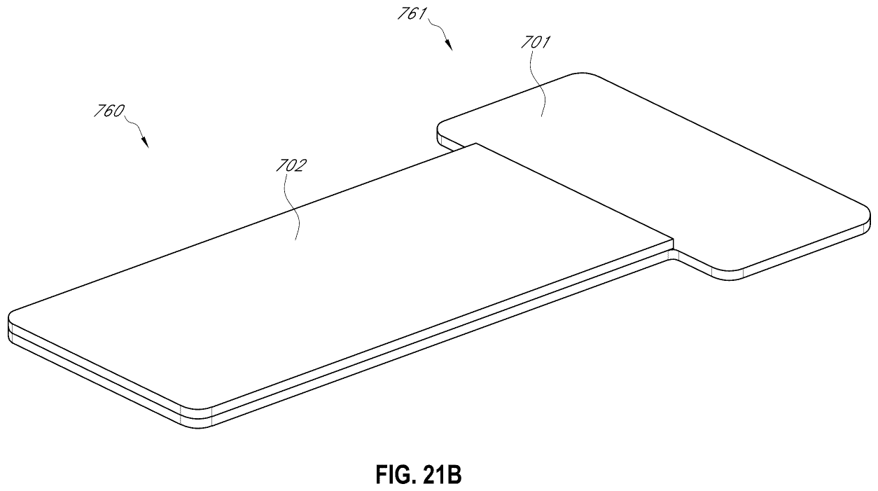

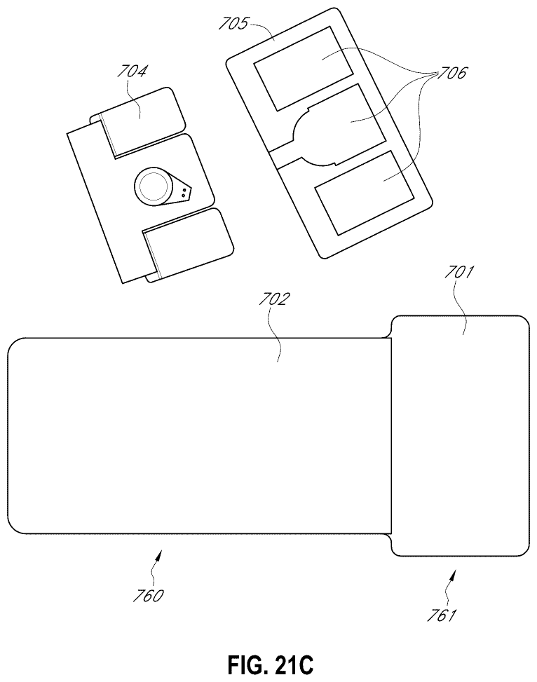

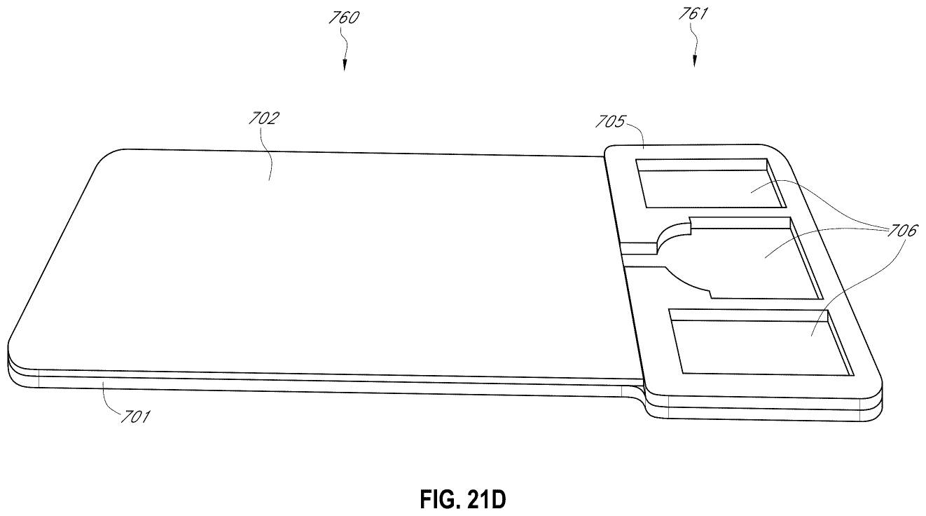

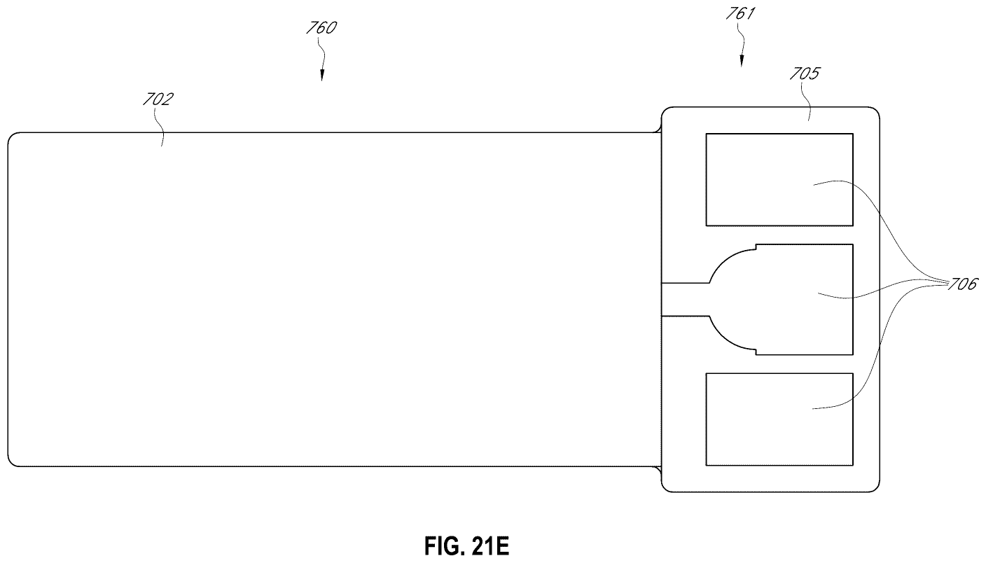

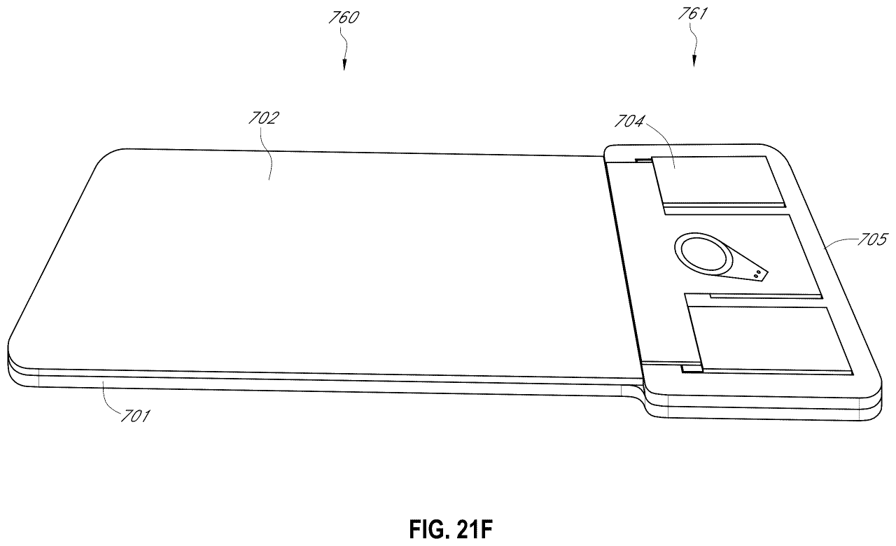

The dressing layers can be assembled with various material layers in several configurations. In some embodiments, the electronics unit can be assembled within the wound dressing adjacent to the absorbent layer as described above. FIGS. 21A-21F illustrate an embodiment of a wound dressing incorporating an electronics unit resting in a cradle. In alternative embodiments, the dressing layers described with reference to FIGS. 21A-21F can incorporate an electronics unit within an electronics cassette as described with reference to FIGS. 5A-5E, 7A-7D, 8A-8C, 9A-9D, and 10 herein. In such embodiments, the electronics cassette can be used instead of the electronics cradle described with reference to FIGS. 21A-21F.

FIG. 21A illustrates an embodiment of a spacer layer 701. The spacer layer can be provided over a wound contact layer (not shown). The wound contact layer is placed in contact with the wound as described herein.

As illustrated in FIGS. 21A-21F, the wound dressing layers can include an absorbent area 760 and an electronics area 761. In some embodiments, the electronics area 761 can include the electronics unit 704. In some embodiments, the absorbent area 760 can include the absorbent material 702 and can be positioned over the wound area to absorb fluid from the wound within the absorbent material or pad 702. FIGS. 21B and 21C illustrates an embodiment of the spacer layer 701 with an absorbent layer 702 provided over the absorbent area 706 of the spacer layer 701. In FIGS. 21B-21C, the spacer layer 701 in the electronics area 761 is visible while the spacer layer 701 in the absorbent area 760 is obscured by the overlying absorbent layer 702.

FIG. 21C illustrates an embodiment of an electronics unit 704 and a cradle 705 shown removed from the wound dressing layers. The cradle 705 include recesses, cutout, or slots 706 within the cradle. The recesses 706 are shaped to fit portions of the electronics unit. The recesses 706 can be shaped in a complementary shape that matches the shape of portions of the underside of the electronics unit 704 similar to the electronics unit 67 shown and described with reference to FIGS. 2, 3A-3D, and 4A-4B. For example, the recesses 706 can be the shape of the batteries and pump assembly as shown in FIGS. 21C-21E. FIGS. 21D-21F illustrate an embodiment of a wound dressing with the cradle 705 positioned over the spacer layer 701 of the electronics area 761. The cradle 705 can be positioned adjacent to the absorbent layer 702. FIG. 21F illustrates an embodiment of the wound dressing with the electronics unit 704 within the recess 706 of the electronics cradle 705. The dressing layers and components shown in FIG. 21F can be enclosed in a wound contact layer (not shown) positioned below the spacer layer 701 and a cover layer (not shown) positioned above the absorbent layer 702 and electronics 704 within the cradle 705. The wound contact layer and cover layer can be sealed at a perimeter enclosing the dressing components.

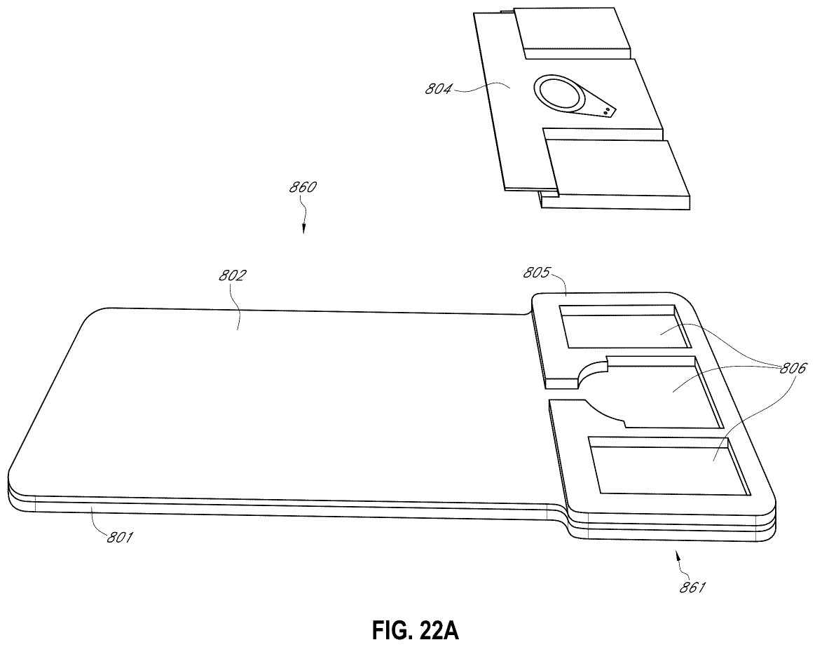

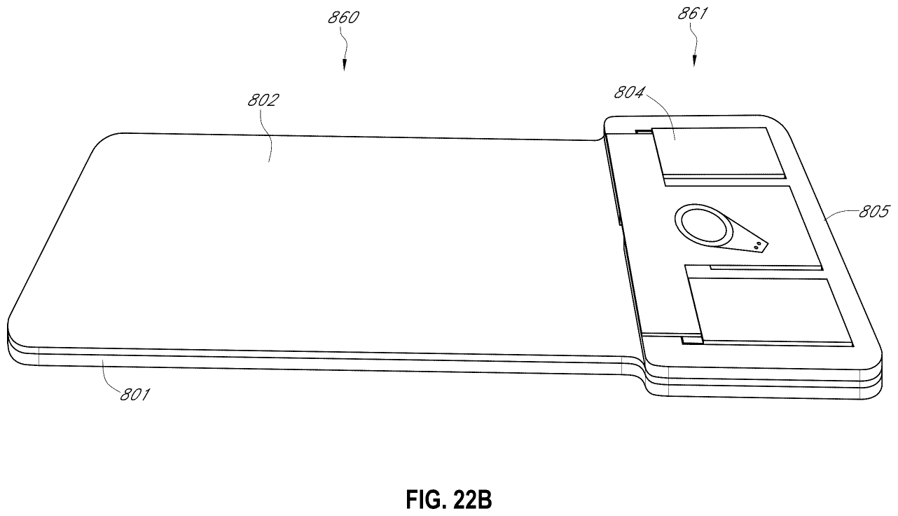

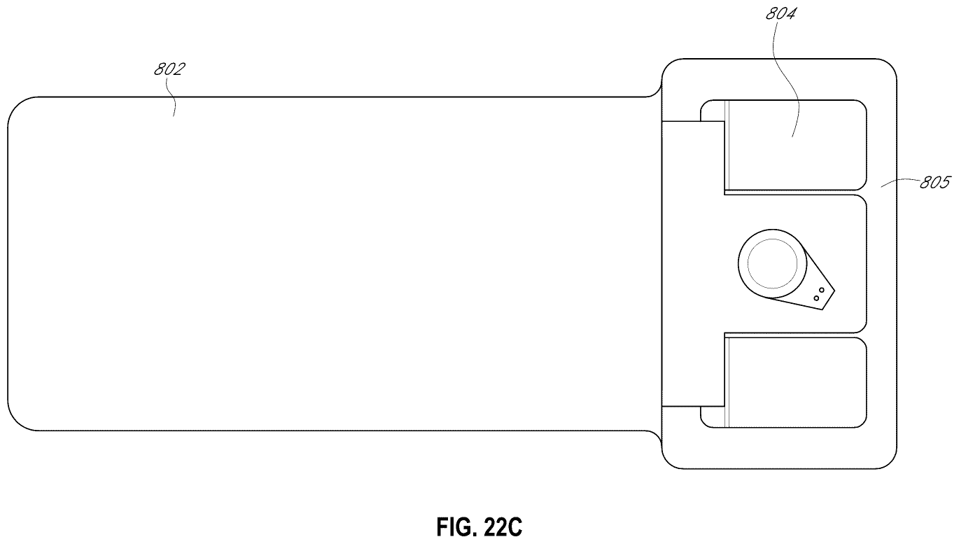

FIGS. 22A-22C illustrate another embodiment of a wound dressing incorporating an electronics unit 804 and cradle 805 in the wound dressing. In alternative embodiments, the dressing layers described with reference to FIGS. 22A-22C can incorporate an electronics unit within an electronics cassette as described with reference to FIGS. 5A-5E, 7A-7D, 8A-8C, 9A-9D, and 10 herein. In such embodiments, the electronics cassette can be used instead of the electronics cradle described with reference to FIGS. 22A-22C.

FIG. 22A illustrates an embodiment of a wound dressing including a spacer layer 801, absorbent layer 802, and cradle 805. As illustrated in FIGS. 22A-22C, the wound dressing layers can include an absorbent area 860 and an electronics area 861. In some embodiments, the electronics area 861 can include the electronics unit 804. In some embodiments, the absorbent area 860 can include the absorbent material 802 and can be positioned over the wound area to absorb fluid from the wound within the absorbent material or pad 802.

The absorbent layer 802 as shown in FIGS. 22A-22C can be positioned above the spacer layer 801 in the absorbent area 860 and the electronics area 861 and spans the length of the dressing. The cradle 805 can be positioned above the absorbent layer 802 in the electronics area 861. FIG. 22A illustrates the recesses 806 in the cradle 805 similar to the recesses described herein with reference to FIGS. 21A-21F. FIGS. 22B and 22C illustrate the electronics unit 804 positioned within the recesses 806 of the cradle. The dressing layers and components shown in FIGS. 22B and 22C can be enclosed in a wound contact layer (not shown) positioned below the spacer layer 801 and a cover layer (not shown) positioned above the absorbent layer 802 and electronics 804 within the cradle 805. The wound contact layer and cover layer can be sealed at a perimeter enclosing the dressing components.

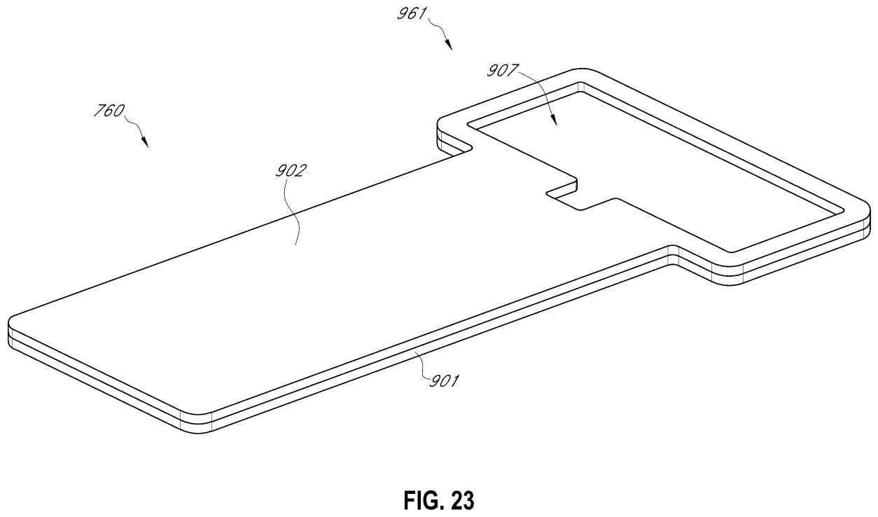

FIG. 23 illustrates an absorbent layer 902 provided over the entire length of the spacer layer 901. As illustrated in FIG. 23, the wound dressing layers can include an absorbent area 960 and an electronics area 961. In some embodiments, the electronics area 961 can include the electronics unit. In some embodiments, the absorbent area 960 can include the absorbent material 902 and can be positioned over the wound area to absorb fluid from the wound within the absorbent material or pad 902. The absorbent layer has one recess, cutout, or slot 907 in the portion of the absorbent layer 902 located in the electronics area 961. The spacer layer 901 is visible in the recess 907 of the absorbent layer 902. In some embodiments, as shown in FIG. 23 the absorbent material 902 spans the length of the dressing and can include a single aperture 907 in the absorbent material 902. In some embodiments, the electronics cradle or electronics cassette described herein can be positioned within the single aperture 907 in the absorbent material or absorbent layer 902 of the dressing. In such embodiments, an electronic unit can be positioned within the electronics cradle or electronics cassette as described herein. The dressing layers and components shown in FIG. 23 can be enclosed in a wound contact layer (not shown) positioned below the spacer layer 901 and a cover layer (not shown) positioned above the absorbent layer 902 and electronics cradle or cassette. The wound contact layer and cover layer can be sealed at a perimeter enclosing the dressing components.

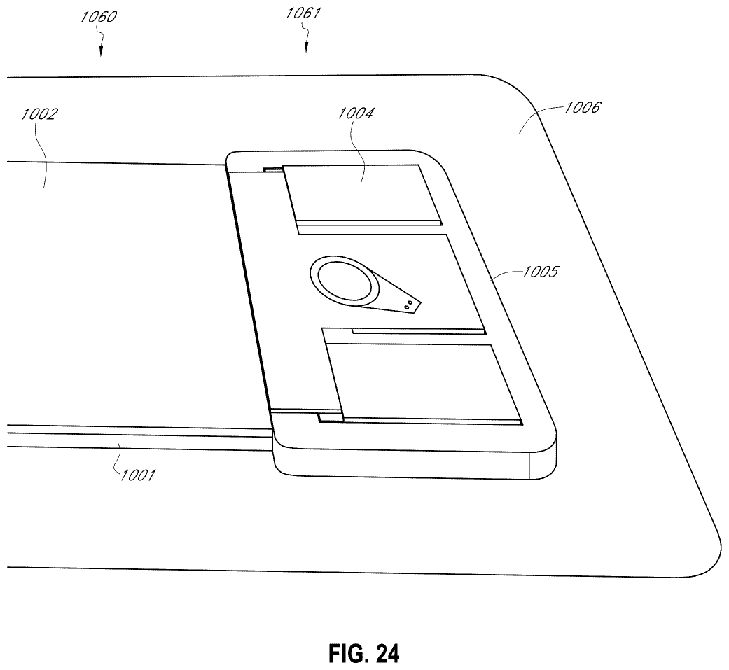

FIG. 24 illustrates an embodiment of a wound dressing incorporating an electronics unit resting in a cradle 1005. In alternative embodiments, the dressing layers described with reference to FIG. 24 can incorporate an electronics unit within an electronics cassette as described with reference to FIGS. 5A-5E, 7A-7D, 8A-8C, 9A-9D, and 10 herein. In such embodiments, the electronics cassette can be used instead of the electronics cradle described with reference to FIG. 24. As illustrated in FIG. 24, the wound dressing layers can include an absorbent area 1060 and an electronics area 1061. In some embodiments, the electronics area 1061 can include the electronics unit 1004 and electronics cradle 1005. In some embodiments, the absorbent area 1060 can include the absorbent material 1002 and can be positioned over the wound area to absorb fluid from the wound within the absorbent material or pad 1002. FIG. 24 illustrates a spacer layer 1001 and an absorbent layer 1002 positioned adjacent to a cradle 1005 and electronics unit 1004. The dressing configuration illustrated in FIG. 24 is similar to the dressing described with reference to FIGS. 21A-21F. However, the spacer layer 1001 as shown in FIG. 24 does not extend the entire length of the dressing. Instead, the spacer layer 1001 only extends to an edge of the cradle 1005 and the cradle is positioned to be in direct contact with an underlying wound contact layer 1006. As illustrated in FIG. 24, the spacer layer 1001 and absorbent layer 1002 are positioned in the absorbent area 1060 and do not extend to the electronics area 1061. The cradle 1005 and electronics unit 1004 are positioned in the electronics area 1061. FIG. 24 illustrates the layers of a wound dressing and electronics area shown with the wound contact layer 1006 and without the cover layer. The dressing layers and components shown in FIG. 24 can be enclosed in a wound contact layer 1006 positioned below the spacer layer and cradle and a cover layer (not shown) positioned above the absorbent layer and electronics unit 1004 within the cradle 1005. The wound contact layer 1006 and cover layer can be sealed at a perimeter enclosing the dressing components.

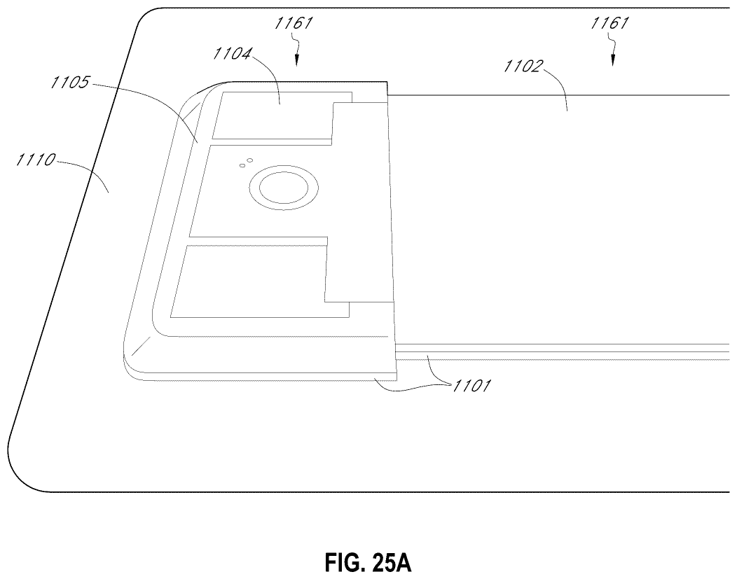

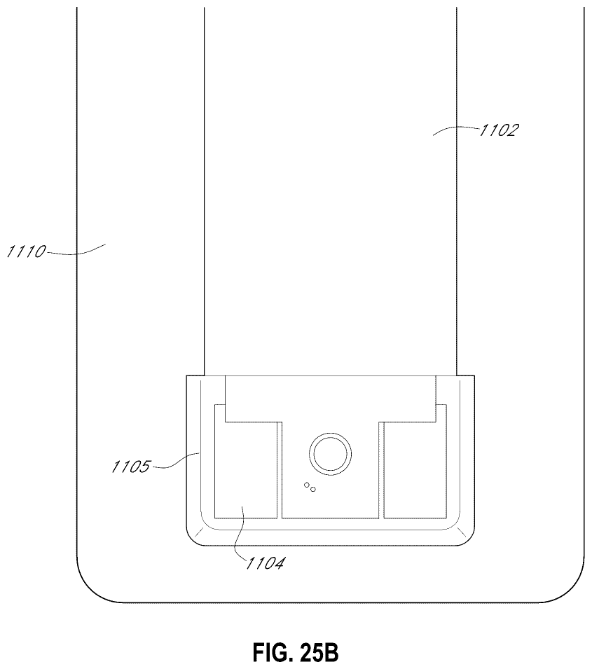

FIGS. 25A-25B illustrate embodiments of an assembled wound dressing with the dressing layers enclosed between a wound contact layer (not shown) and a cover layer 1110. As illustrated in FIGS. 25A-25B, the wound dressing layers can include an absorbent area 1160 and an electronics area 1161. In some embodiments, the electronics area 1161 can include the electronics unit 1104 and electronics cradle 1105. In some embodiments, the absorbent area 1160 can include the absorbent material 1102 and can be positioned over the wound area to absorb fluid from the wound within the absorbent material or pad 1102. The wound dressing can include a spacer layer 1101 as illustrated in FIG. 25A. The dressing layers and components shown in FIGS. 25A-25B can be enclosed in a wound contact layer (not shown) positioned below the spacer layer 1101 and a cover layer 1110 positioned above the absorbent layer 1102 and electronics unit 1104 within the cradle 1105. The wound contact layer and cover layer 1110 can be sealed at a perimeter enclosing the dressing components.

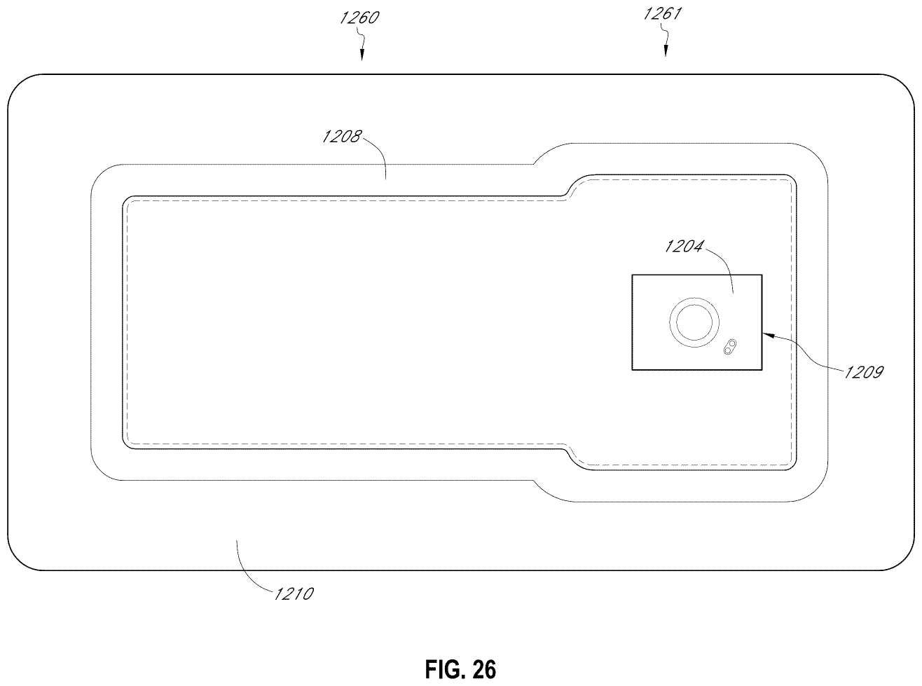

FIG. 26 illustrates an embodiment of an assembled wound dressing similar to the dressing shown in FIGS. 25A-25B. However, the wound dressing of FIG. 26 includes an additional masking or obscuring layer 1208 positioned above the dressing layers and below the cover layer 1210. As illustrated in FIG. 26, the wound dressing layers can include an absorbent area 1260 and an electronics area 1261. In some embodiments, the electronics area 1261 can include the electronics unit 1204. In some embodiments, the absorbent area 1260 can include the absorbent material and can be positioned over the wound area to absorb fluid from the wound within the absorbent material or pad. In some embodiments, the masking or obscuring layer 1208 can be positioned above the absorbent layer and electronics 1204 and below the cover layer 1210. In some embodiments, the masking or obscuring layer 1208 can include an aperture 1209 that allows the electronics 1204 below the masking or obscuring layer 1208 to be visible as shown in FIG. 26. In some embodiments, the masking or obscuring layer 1208 can be an opaque material that does not allow the wound exudate or other fluid to be visible from a top view of the wound dressing. In some embodiments, the masking or obscuring layer can span the entire length of the absorbent area 1260 and electronics area 1261. In some embodiments, the masking or obscuring layer can have a larger perimeter than the spacer layer, absorbent layer, and electronics unit, cradle, or cassette and can overborder the absorbent material and electronics unit, cradle, or cassette as shown in FIG. 26.

Disclosed embodiments relate to apparatuses and methods for wound treatment. In some embodiments, a wound dressing apparatus can comprise a wound contact layer, a spacer layer, an absorbent layer positioned on the spacer layer, an electronics unit comprising a negative pressure source and/or electronic components, wherein the absorbent layer comprises a recess configured to receive the electronics unit and the absorbent layer is configured to be in fluid communication with the electronics unit, and a cover layer configured to cover and form a seal over the wound contact layer, the spacer layer, the absorbent layer, and the electronics unit.

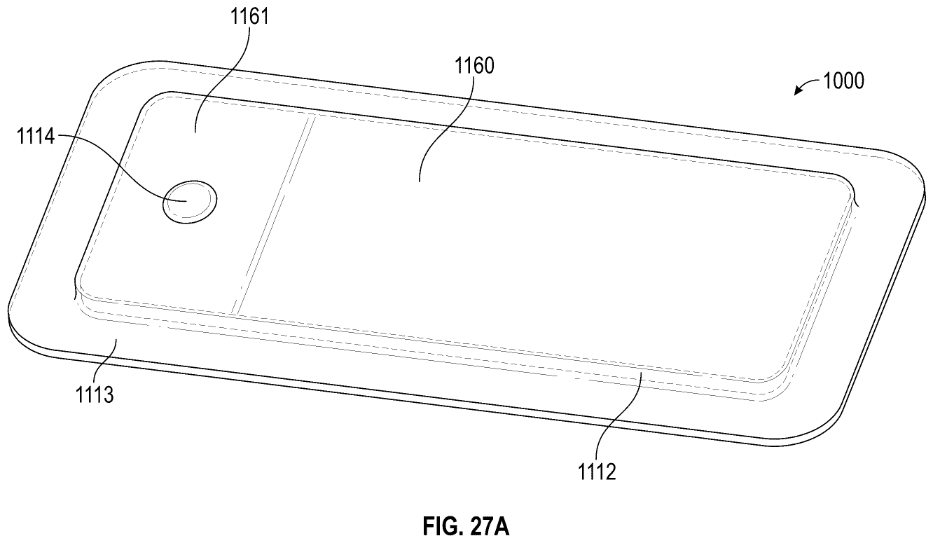

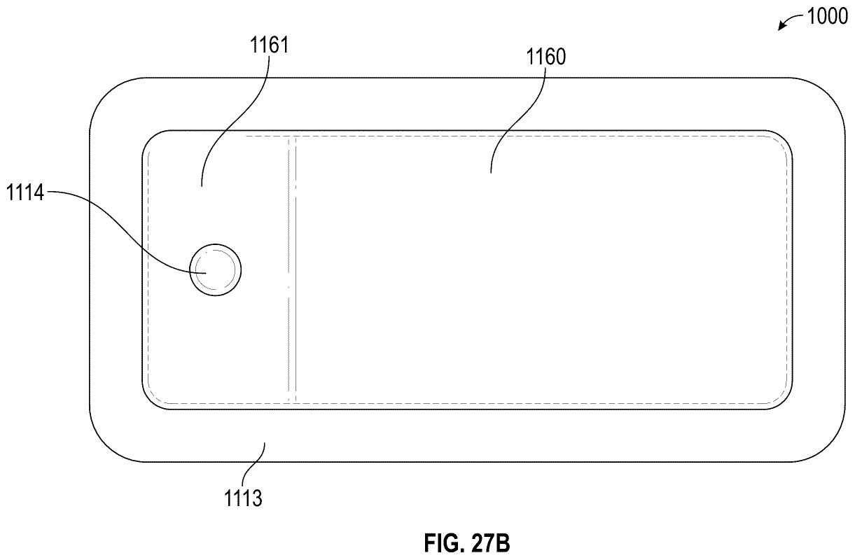

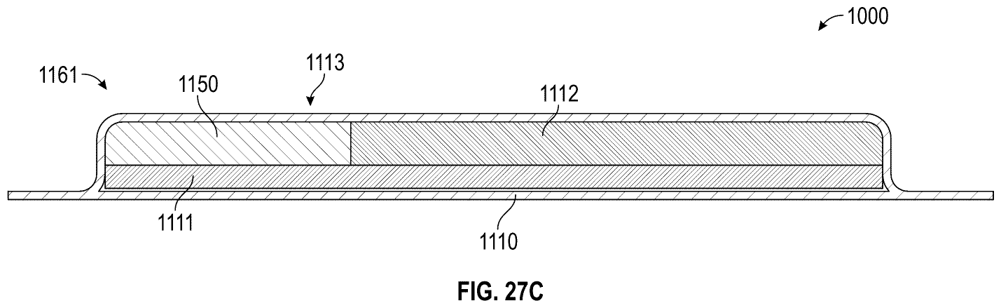

In some embodiments, a source of negative pressure (such as a pump) and some or all other components of the TNP system, such as power source(s), sensor(s), connector(s), user interface component(s) (such as button(s), switch(es), speaker(s), screen(s), etc.) and the like, can be integral with the wound dressing. The wound dressing can include various material layers described here and described in further detail in International Application No. PCT/EP2017/055225, filed Mar. 6, 2017, entitled WOUND TREATMENT APPARATUSES AND METHODS WITH NEGATIVE PRESSURE SOURCE INTEGRATED INTO WOUND DRESSING. The material layers can include a wound contact layer, one or more absorbent layers, one or more spacer or transmission layers, and a backing layer or cover layer covering the one or more absorbent and spacer or transmission layers. The wound dressing can be placed over a wound and sealed to the wound with the pump and/or other electronic components contained under the cover layer within the wound dressing. In some embodiments, the dressing can be provided as a single article with all wound dressing elements (including the pump) pre-attached and integrated into a single unit. In some embodiments, a periphery of the wound contact layer can be attached to the periphery of the cover layer enclosing all wound dressing elements as illustrated in FIG. 27A-27C.

In some embodiments, the pump and/or other electronic components can be configured to be positioned adjacent to or next to the absorbent and/or transmission layers so that the pump and/or other electronic components are still part of a single article to be applied to a patient. In some embodiments, with the pump and/or other electronics positioned away from the wound site. FIGS. 27A-27C illustrates a wound dressing incorporating the source of negative pressure and/or other electronic components within the wound dressing. FIGS. 27A-27C illustrates a wound dressing 1000 with the pump and/or other electronics positioned away from the wound site. The wound dressing can include an electronics area 1161 and an absorbent area 1160. The dressing can comprise a wound contact layer 1110 (not shown in FIGS. 27A-27B) and a moisture vapor permeable film or cover layer 1113 positioned above the contact layer and other layers of the dressing. The wound dressing layers and components of the electronics area as well as the absorbent area can be covered by one continuous cover layer 1113 as shown in FIGS. 27A-27C.

The dressing can comprise a wound contact layer 1110, a transmission layer 1111, an absorbent layer 1112, a moisture vapor permeable film or cover layer 1113, 1113 positioned above the wound contact layer, transmission layer, absorbent layer, or other layers of the dressing. The wound contact layer can be configured to be in contact with the wound. The wound contact layer can include an adhesive on the patient facing side for securing the dressing to the surrounding skin or on the top side for securing the wound contact layer to a cover layer or other layer of the dressing. In operation, the wound contact layer can be configured to provide unidirectional flow so as to facilitate removal of exudate from the wound while blocking or substantially preventing exudate from returning to the wound.

The wound contact layer 1110 can be a polyurethane layer or polyethylene layer or other flexible layer which is perforated, for example via a hot pin process, laser ablation process, ultrasound process or in some other way or otherwise made permeable to liquid and gas. The wound contact layer 1110 has a lower surface and an upper surface. The perforations preferably comprise through holes in the wound contact layer 1110 which enable fluid to flow through the layer 1110. The wound contact layer 1110 helps prevent tissue ingrowth into the other material of the wound dressing. Preferably, the perforations are small enough to meet this requirement while still allowing fluid to flow therethrough. For example, perforations formed as slits or holes having a size ranging from 0.025 mm to 1.2 mm are considered small enough to help prevent tissue ingrowth into the wound dressing while allowing wound exudate to flow into the dressing. In some configurations, the wound contact layer 1110 may help maintain the integrity of the entire dressing 1000 while also creating an air tight seal around the absorbent pad in order to maintain negative pressure at the wound.

Some embodiments of the wound contact layer 1110 may also act as a carrier for an optional lower and upper adhesive layer (not shown). For example, a lower pressure sensitive adhesive may be provided on the lower surface of the wound dressing 1000 whilst an upper pressure sensitive adhesive layer may be provided on the upper surface of the wound contact layer. The pressure sensitive adhesive, which may be a silicone, hot melt, hydrocolloid or acrylic based adhesive or other such adhesives, may be formed on both sides or optionally on a selected one or none of the sides of the wound contact layer. When a lower pressure sensitive adhesive layer is utilized it may be helpful to adhere the wound dressing 1000 to the skin around a wound site. In some embodiments, the wound contact layer may comprise perforated polyurethane film. The lower surface of the film may be provided with a silicone pressure sensitive adhesive and the upper surface may be provided with an acrylic pressure sensitive adhesive, which may help the dressing maintain its integrity. In some embodiments, a polyurethane film layer may be provided with an adhesive layer on both its upper surface and lower surface, and all three layers may be perforated together.

A layer 1111 of porous or transmission material can be located above the wound contact layer 1110. As used herein, the terms porous material, spacer, and/or transmission layer can be used interchangeably to refer to the layer of material in the dressing configured to distribute negative pressure throughout the wound area. This porous layer, or transmission layer, 1111 allows transmission of fluid including liquid and gas away from a wound site into upper layers of the wound dressing. In particular, the transmission layer 1111 preferably ensures that an open air channel can be maintained to communicate negative pressure over the wound area even when the absorbent layer has absorbed substantial amounts of exudates. The layer 1111 should preferably remain open under the typical pressures that will be applied during negative pressure wound therapy as described above, so that the whole wound site sees an equalized negative pressure. The layer 1111 may be formed of a material having a three dimensional structure. For example, a knitted or woven spacer fabric (for example Baltex 7970 weft knitted polyester) or a non-woven fabric could be used.

The transmission layer assists in distributing negative pressure over the wound site and facilitating transport of wound exudate and fluids into the wound dressing. In some embodiments, the transmission layer can be formed at least partially from a three dimensional (3D) fabric.

In some embodiments, the transmission layer 1111 comprises a 3D polyester spacer fabric layer including a top layer (that is to say, a layer distal from the wound-bed in use) which is a 84/144 textured polyester, and a bottom layer (that is to say, a layer which lies proximate to the wound bed in use) which is a 10 denier flat polyester and a third layer formed sandwiched between these two layers which is a region defined by a knitted polyester viscose, cellulose or the like monofilament fiber. Other materials and other linear mass densities of fiber could of course be used.

Whilst reference is made throughout this disclosure to a monofilament fiber it will be appreciated that a multistrand alternative could of course be utilized. The top spacer fabric thus has more filaments in a yarn used to form it than the number of filaments making up the yarn used to form the bottom spacer fabric layer.

This differential between filament counts in the spaced apart layers helps control moisture flow across the transmission layer. Particularly, by having a filament count greater in the top layer, that is to say, the top layer is made from a yarn having more filaments than the yarn used in the bottom layer, liquid tends to be wicked along the top layer more than the bottom layer. In use, this differential tends to draw liquid away from the wound bed and into a central region of the dressing where the absorbent layer 1112 helps lock the liquid away or itself wicks the liquid onwards towards the cover layer 1113 where it can be transpired.

Preferably, to improve the liquid flow across the transmission layer 1111 (that is to say perpendicular to the channel region formed between the top and bottom spacer layers), the 3D fabric may be treated with a dry cleaning agent (such as, but not limited to, Perchloro Ethylene) to help remove any manufacturing products such as mineral oils, fats or waxes used previously which might interfere with the hydrophilic capabilities of the transmission layer. In some embodiments, an additional manufacturing step can subsequently be carried in which the 3D spacer fabric is washed in a hydrophilic agent (such as, but not limited to, Feran Ice 30 g/l available from the Rudolph Group). This process step helps ensure that the surface tension on the materials is so low that liquid such as water can enter the fabric as soon as it contacts the 3D knit fabric. This also aids in controlling the flow of the liquid insult component of any exudates.

Further, an absorbent layer (such as layer 1112) for absorbing and retaining exudate aspirated from the wound can be utilized. In some embodiments, a superabsorbent material can be used in the absorbent layer 1112. In some embodiments, the absorbent includes a shaped form of a superabsorber layer.

A layer 1112 of absorbent material is provided above the transmission layer 111. The absorbent material, which comprise a foam or non-woven natural or synthetic material, and which may optionally comprise a super-absorbent material, forms a reservoir for fluid, particularly liquid, removed from the wound site. In some embodiments, the layer 1111 may also aid in drawing fluids towards the cover layer 1113.

The material of the absorbent layer 1112 may also prevent liquid collected in the wound dressing from flowing freely within the dressing, and preferably acts so as to contain any liquid collected within the dressing. The absorbent layer 1112 also helps distribute fluid throughout the layer via a wicking action so that fluid is drawn from the wound site and stored throughout the absorbent layer. This helps prevent agglomeration in areas of the absorbent layer. The capacity of the absorbent material must be sufficient to manage the exudates flow rate of a wound when negative pressure is applied. Since in use the absorbent layer experiences negative pressures the material of the absorbent layer is chosen to absorb liquid under such circumstances. A number of materials exist that are able to absorb liquid when under negative pressure, for example superabsorber material. The absorbent layer 1112 may typically be manufactured from ALLEVYN™ foam, Freudenberg 114-224-4 or Chem-Posite™11C-450. In some embodiments, the absorbent layer 1112 may comprise a composite comprising superabsorbent powder, fibrous material such as cellulose, and bonding fibers. In a preferred embodiment, the composite is an airlaid, thermally-bonded composite.

In some embodiments, the absorbent layer 1112 is a layer of non-woven cellulose fibers having super-absorbent material in the form of dry particles dispersed throughout. Use of the cellulose fibers introduces fast wicking elements which help quickly and evenly distribute liquid taken up by the dressing. The juxtaposition of multiple strand-like fibers leads to strong capillary action in the fibrous pad which helps distribute liquid. In this way, the super-absorbent material is efficiently supplied with liquid. The wicking action also assists in bringing liquid into contact with the upper cover layer to aid increase transpiration rates of the dressing.

The wound dressing layers of the electronics area and the absorbent layer can be covered by one continuous cover layer or backing layer 1113. As used herein, the terms cover layer and/or backing layer can be used interchangeably to refer to the layer of material in the dressing configured to cover the underlying dressing layers and seal to the wound contact layer and/or the skin surrounding the wound. In some embodiments, the cover layer can include a moisture vapor permeable material that prevents liquid exudate removed from the wound and other liquids from passing through, while allowing gases through.

The cover layer 1113 is preferably gas impermeable, but moisture vapor permeable, and can extend across the width of the wound dressing 1100. The cover layer 1113, which may for example be a polyurethane film (for example, Elastollan SP9109) having a pressure sensitive adhesive on one side, is impermeable to gas and this layer thus operates to cover the wound and to seal a wound cavity over which the wound dressing is placed. In this way an effective chamber is made between the cover layer 1113 and a wound site where a negative pressure can be established. The cover layer 1113 is preferably sealed to the wound contact layer 1110 in a border region around the circumference of the dressing, ensuring that no air is drawn in through the border area, for example via adhesive or welding techniques. The cover layer 1113 protects the wound from external bacterial contamination (bacterial barrier) and allows liquid from wound exudates to be transferred through the layer and evaporated from the film outer surface. The cover layer 1113 preferably comprises two layers; a polyurethane film and an adhesive pattern spread onto the film. The polyurethane film is preferably moisture vapor permeable and may be manufactured from a material that has an increased water transmission rate when wet. In some embodiments, the moisture vapor permeability of the cover layer increases when the cover layer becomes wet. The moisture vapor permeability of the wet cover layer may be up to about ten times more than the moisture vapor permeability of the dry cover layer.

The electronics area 1161 can include a source of negative pressure (such as a pump) and some or all other components of the TNP system, such as power source(s), sensor(s), connector(s), user interface component(s) (such as button(s), switch(es), speaker(s), screen(s), etc.) and the like, that can be integral with the wound dressing. For example, the electronics area 1161 can include a button or switch 1114 as shown in FIGS. 27A-27B. The button or switch 1114 can be used for operating the pump (e.g., turning the pump on/off).

The absorbent area 1160 can include an absorbent material 1112 and can be positioned over the wound site. The electronics area 1161 can be positioned away from the wound site, such as by being located off to the side from the absorbent area 1160. The electronics area 1161 can be positioned adjacent to and in fluid communication with the absorbent area 1160 as shown in FIGS. 27A-27C. In some embodiments, each of the electronics area 1161 and absorbent area 160 may be rectangular in shape and positioned adjacent to one another. In some embodiments, the electronics unit can be within absorbent material in the electronics area 1160 of the dressing as described herein. As illustrated in FIG. 27C, the electronics unit can be positioned within the absorbent material but off to the side of the absorbent area.

In some embodiments, additional layers of dressing material can be included in the electronics area 1161, the absorbent area 1160, or both areas. In some embodiments, the dressing can comprise one or more transmission layers and/or one or more absorbent layer positioned above the wound contact layer 1110 and below the cover layer 1113 of the dressing.

In some embodiments, the electronics area 1161 of the dressing can comprise electronic components 1150. In some embodiments, the electronics area 1161 of the dressing can comprise a plurality of layers of transmission material and/or absorbent material and electronic components 1150 can be embedded within the plurality of layers of transmission material and/or absorbent material. The layers of transmission or absorbent material can have recesses or cut outs to embed the electronic components 1150 within whilst providing structure to prevent collapse. The electronic components 1150 can include a pump, power source, controller, and/or an electronics package.

A pump exhaust can be provided to exhaust air from the pump to the outside of the dressing. The pump exhaust can be in communication with the electronics area 1161 and the outside of the dressing.

As used herein the upper layer, top layer, or layer above refers to a layer furthest from the surface of the skin or wound while the dressing is in use and positioned over the wound. Accordingly, the lower surface, lower layer, bottom layer, or layer below refers to the layer that is closest to the surface of the skin or wound while the dressing is in use and positioned over the wound. Additionally, the layers can have a proximal wound-facing face referring to a side or face of the layer closest to the skin or wound and a distal face referring to a side or face of the layer furthest from the skin or wound.

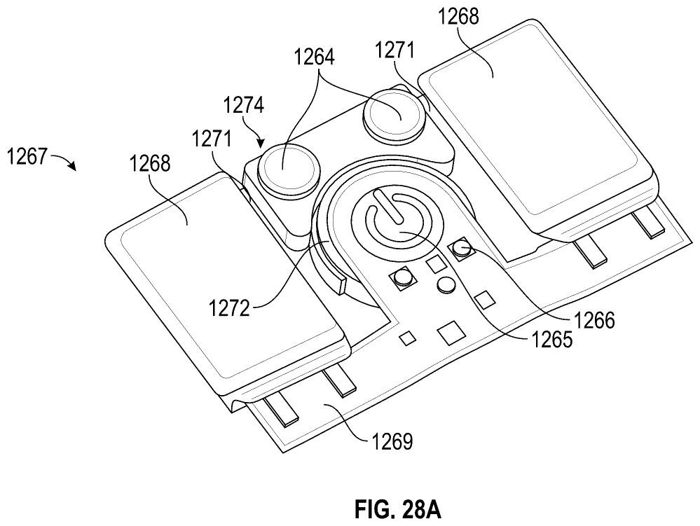

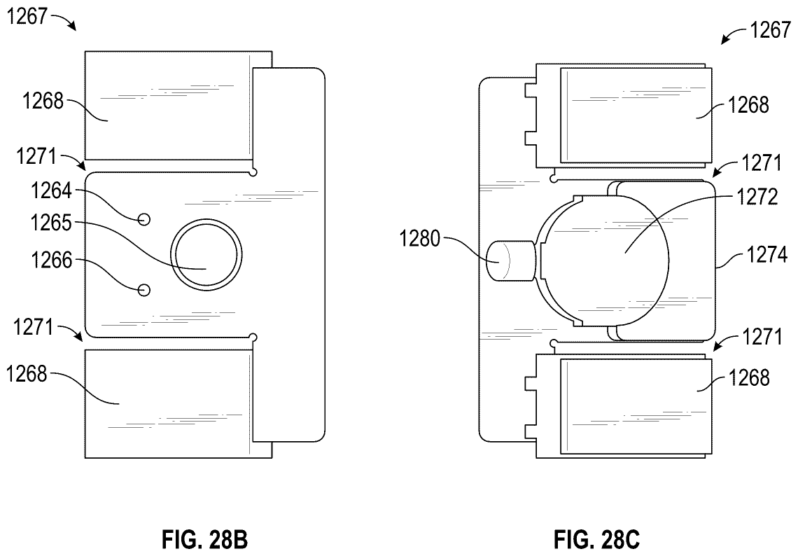

FIG. 27A-27C illustrates a wound dressing apparatus incorporating the pump and/or other electronic components within the wound dressing and offset from the absorbent layer. In some embodiments, as shown in FIG. 27C, the absorbent area 1160 comprises a transmission layer 1111 positioned above the wound contact layer 1110. An absorbent layer 1112 can be provided above the transmission layer 1111. In some embodiments, the electronics area 1161 can include an electronics unit (shown in FIGS. 28A-28C). In some embodiments, the electronics unit is provided directly over the wound contact layer. In other embodiments, the electronics unit can be placed above a layer of wicking material, absorbent material, or transmission material that sits above the wound contact layer 1110 of the dressing. For example, as shown in FIG. 27C, the electronics unit 1150 may be positioned over the transmission layer 1111. In some embodiments, the transmission layer 1111 can be a single layer of material extending below the electronics unit 1150 and the absorbent material 1112. Thus, in some embodiments, the transmission layer 1111 extends continuously through the absorbent area 1160 and the electronics area 1161. In alternative embodiments, the transmission layer below the electronics unit can be a different transmission layer than the transmission layer below the absorbent material 1112. The transmission layer 1111, absorbent material 1112, and electronics unit 1150 can be covered with a cover layer 1113 that seals to a perimeter of the wound contact layer 1110 as shown in FIGS. 27A-27C.

The electronics area 1161 can include an electronics unit 1150 positioned below the cover layer 1113 of the dressing. In some embodiments, the electronics unit can be surrounded by a material to enclose or encapsulate a negative pressure source and electronics components by surrounding the electronics. In some embodiments, this material can be a casing. In some embodiments, the electronics unit can be encapsulated or surrounded by a protective coating, for example, a hydrophobic coating as described herein. The electronics unit can be in contact with the dressing layers in the absorbent area 1160 and covered by the cover layer 1113. As used herein, the electronics unit includes a lower or wound facing surface that is closest to the wound and an opposite, upper surface, furthest from the wound when the wound dressing is placed over a wound.

FIG. 28A illustrate an embodiment of the electronics unit 1267. FIG. 28A illustrates an embodiment of a pump and electronics unit 1267 that can be incorporated into a wound dressing. The electronics unit 1267 of FIG. 28A is shown without an electronics casing or other dressing material. FIGS. 28B-28C illustrate embodiments of the pump and electronics unit 1267. FIG. 28B illustrates the top view of the electronics unit. FIG. 28C illustrates a bottom or wound facing surface of the electronics unit.

As illustrated in FIG. 28A-28B, the electronics unit 1267 can include single button 1265 on the upper surface of the unit. The single button 1265 can be used as an on/off button or switch to stop and start operation of the pump and/or electronic components. The switch 1265 can be a dome type switch configured to sit on the top of the pump. Because the switch is situated within the dressing the cover layer can be easily sealed around or over the switch. In some embodiments, the cover layer can have an opening or hole positioned above the switch. The cover layer can be sealed to the outer perimeter of the switch 1265 to maintain negative pressure under the wound cover. The switch can be placed on any surface of the electronics unit and can be in electrical connection with the pump.

The electronics unit 1267 can also include one or more vents or exhausts 1264 for the pump outlet. However, the vent or exhaust 1264 is positioned at the outlet of the pump and extending to the upper surface of the electronics unit. As shown in FIG. 28A, the pump outlet exhaust 1264 is attached to the outlet of the pump and provides communication with the top surface of the dressing. In some embodiments, the exhaust 1264 can be attached to the outlet end of the pump and can extend out from the pump at a 90-degree angle from the pump orientation to communicate with the top surface of the dressing. The exhaust 1264 can include an antibacterial membrane and a non-return valve. The exhausted air from the pump can pass through the pump outlet and exhaust mechanism 1274. In some embodiments, the cover layer 1113 can include apertures or holes positioned above the exhaust vents 1264 and/or membrane. The cover layer 1113 can be sealed to the outer perimeter of the exhaust vents 1264 to maintain negative pressure under the wound cover 1113. In some embodiments, the exhausted air can be exhausted through the gas permeable material or moisture vapor permeable material of the cover layer. In some embodiments, the cover layer does not need to contain apertures or holes over the exhaust and the exhausted air is expelled through the cover layer. In some embodiments, the pump outlet mechanism 1274 can be a custom part formed to fit around the pump as shown in FIG. 28A-28C. The electronic unit 1267 can include a pump inlet protection mechanism 1280 (shown in FIG. 28C) positioned on the portion of the electronic unit closest to the absorbent area and aligned with the inlet of the pump 1272. The pump inlet protection mechanism is positioned between the pump inlet and the absorbent area or absorbent layer of the dressing. The pump inlet protection mechanism can be formed of a hydrophobic material to prevent fluid from entering the pump.

In some embodiments, the upper surface of the electronics unit can include one or more indicators 1266 for indicating a condition of the pump and/or level of pressure within the dressing. The indicators can be small LED lights or other light source that are visible through the dressing material or through holes in the dressing material above the indicators. The indicators can be green, yellow, red, orange, or any other color. For example, there can be two lights, one green light and one orange light. The green light can indicate the device is working properly and the orange light can indicate that there is some issue with the pump (e.g. dressing leak, saturation level of the dressing, and/or low battery).

FIG. 28A-28C illustrates an embodiment of a pump and electronics unit 1267. The electronics unit 1267 can include a pump 1272 and one or more batteries 1268 or other power source to power the pump 1272 and other electronics. The pump can operate at about 27 volts or about 30 volts. The two batteries can allow for a more efficient voltage increase (6 volts to 30 volts) than would be possible with a single battery.

The batteries 1268 can be in electrical communication with a flexible circuit board 1276. In some embodiments, one or more battery connections are connected to a surface of the flexible circuit board 1276. In some embodiments, the flexible circuit board can have other electronics incorporated within. For example, the flexible circuit board may have various sensors including, but not limited to, one or more pressure sensors, temperature sensors, optic sensors and/or cameras, and/or saturation indicators.

In such embodiments, the components of the electronics unit 1267 may include a protective coating to protect the electronics from the fluid within the dressing. The coating can provide a means of fluid separation between the electronics unit 1267 and the absorbent materials of the dressing. The coating can be a hydrophobic coating including, but not limited to, a silicone coating or polyurethane coating. The pump inlet component can be used to protect the pump from fluid on the inlet and the pump outlet mechanism can include a non-return valve that protects fluid from entering the outlet as described in more detail with reference to PCT International Application No. PCT/EP2017/055225, filed Mar. 6, 2017, titled WOUND TREATMENT APPARATUSES AND METHODS WITH NEGATIVE PRESSURE SOURCE INTEGRATED INTO WOUND DRESSING and PCT International Application No. PCT/EP2017/059883, filed Apr. 26, 2017, titled WOUND DRESSINGS AND METHODS OF USE WITH INTEGRATED NEGATIVE PRESSURE SOURCE HAVING A FLUID INGRESS INHIBITION COMPONENT, which is hereby incorporated by reference in its entirety.

The electronics unit 1267 includes one or more slits, grooves or recesses 1271 in the unit between the pump and the two batteries. The slits, grooves or recesses 1271 can allow for the electronics unit 1267 to be flexible and conform to the shape of the wound. The unit 1267 can have two parallel slits, grooves or recesses 1271 forming three segments of the electronics unit 1267. The slits, grooves or recesses 1271 of the unit 1267 create hinge points or gaps that allows for flexibility of the electronics unit at that hinge point. The pump exhaust vents 1264, switch 1265, and indicator 1266 are shown on the top surface surrounded by the electronics unit 1267. As illustrated, one embodiment of the electronics unit 1267 has two hinge points to separate the unit into three regions or panels, for example one to contain one battery, one to contain the pump, and one to contain another battery. In some embodiments, the slits, grooves or recesses may extend parallel with a longitudinal axis of the dressing that extends along the length of the dressing through the electronics area of the dressing through the absorbent area of the dressing.

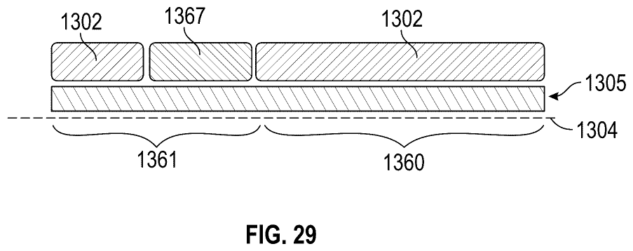

FIG. 29 illustrates an embodiment of a wound dressing incorporating an electronics unit 1367 within the dressing. In some embodiments, the wound dressing can include a wound contact layer 1304. The dressing can also include a transmission layer 1305 which may be made of a 3D material above the wound contact layer. In some embodiments, the electronics sub assembly or electronics unit 1367 can be embedded in an aperture or hole in an absorbent pad 1302 towards one end of the dressing, as depicted in FIG. 29. As shown in the cross sectional view of the wound dressing layers in FIG. 29, the absorbent material 1302 can be positioned on both sides of the electronic components 1367.

In some embodiments, the absorbent components in the absorbent area 1360 can be adjacent to or offset from the electronics unit 1367 in the electronics area 1361 as illustrated in FIG. 29. In some embodiments, the absorbent components and electronics components can be overlapping but offset. For example, a portion of the electronics area 1361 can overlap the absorbent area 1360, for example overlapping the superabsorber layer, but the electronics area 1361 is not completely over the absorbent area 1360. Therefore, a portion of the electronics area can be offset from the absorbent area. The dressing layer and electronic components can be enclosed in a wound contact layer 1304 positioned below the lower most layer and a cover layer (not shown) positioned above the absorbent layer and electronics. The wound contact layer and cover layer can be sealed at a perimeter enclosing the dressing components. In some embodiments, the cover layer can be in direct physical contact with the absorbent material, and/or the electronics unit. In some embodiments, the cover layer can be sealed to a portion of the electronics unit and/or casing, for example, in areas where holes or apertures are used to accommodate the electronic components (e.g. a switch and/or exhaust).

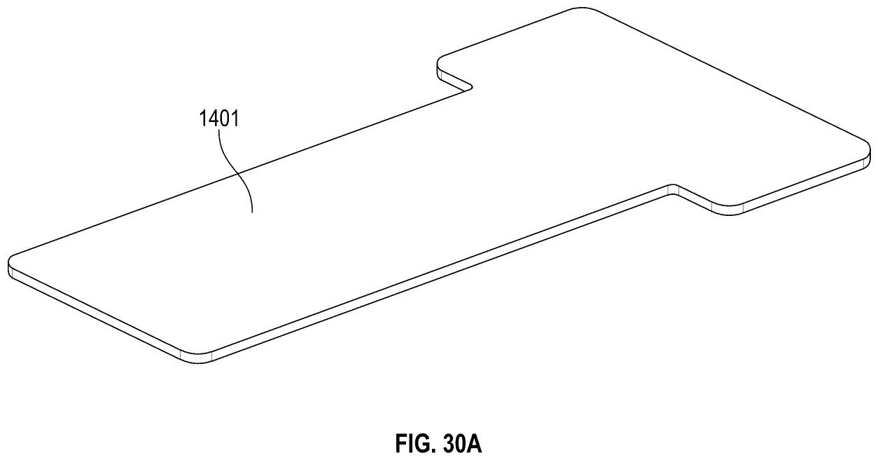

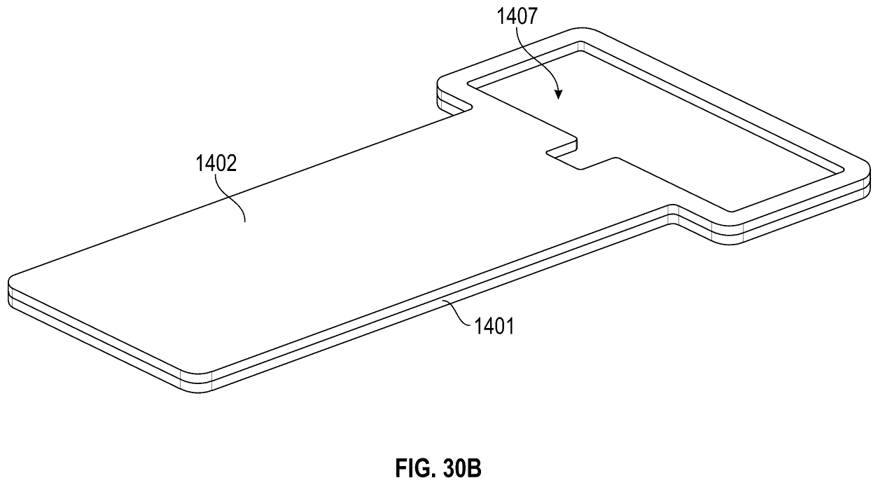

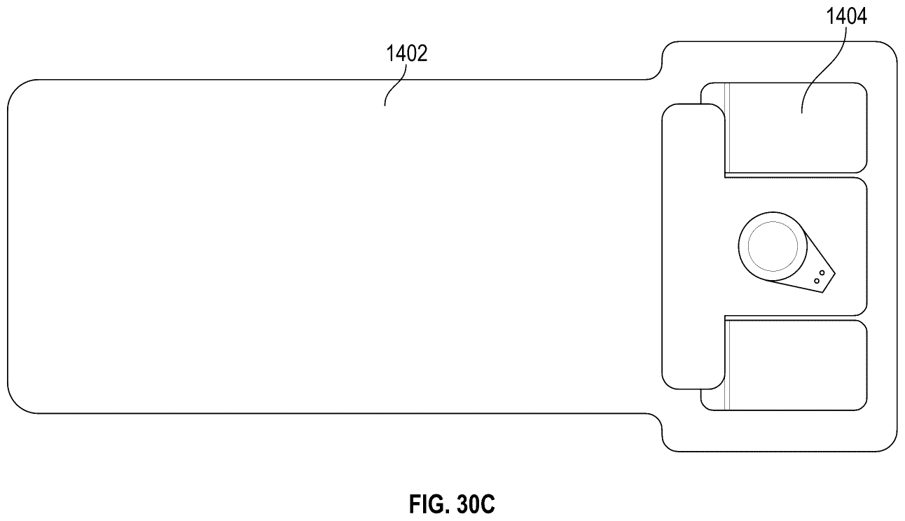

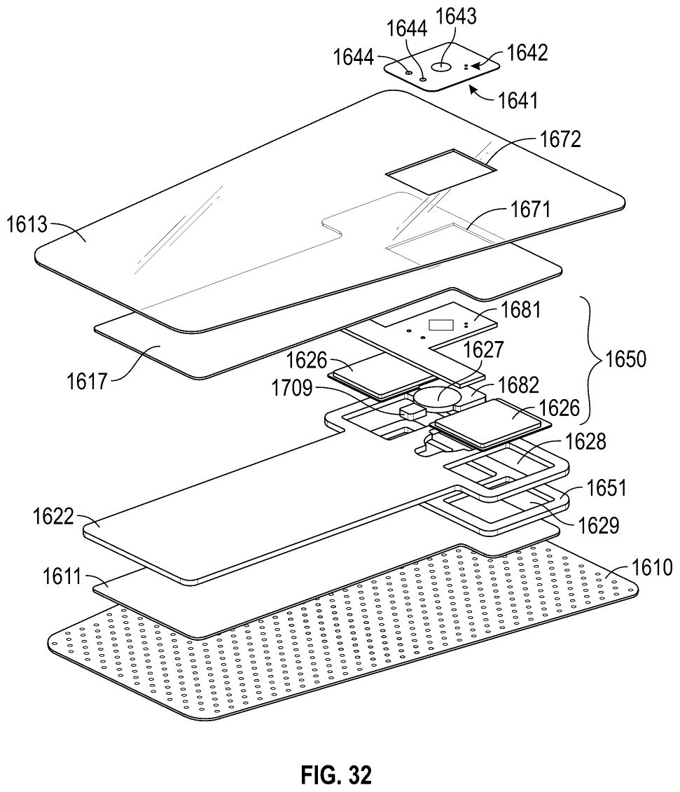

FIGS. 30A-30C illustrate an embodiment of a wound dressing incorporating an electronics unit resting in the absorbent layer. FIG. 30A illustrates a transmission layer 1401. FIG. 30B illustrates an absorbent layer 1402 provided over the entire length of the transmission layer 1401. The absorbent layer has one recess, cutout, or slot 1407 in the portion of the absorbent layer 1402 located in the electronics area. In FIG. 30B, the transmission layer 1401 is visible in the recess 1407 of the absorbent layer 1402. The recess 1407 is spaced and sized to fit the outer perimeter of the batteries and pump assembly of the electronics unit 1404 (as shown in FIG. 30C) in one recess. In some embodiments, the recess in the absorbent layer can include multiple recesses that are sized to fit individual components of the electronics unit 1404, for example, the batteries and pump assembly as illustrated in embodiments described with reference to FIGS. 32, 33A-33C, and 34A-34F. FIG. 30C illustrates the electronics unit 1404 positioned within the recess 1407 of the absorbent layer 1402. The dressing layers and components shown in FIG. 30C can be enclosed in a wound contact layer (not shown) positioned below the transmission layer and a cover layer (not shown) positioned above the absorbent layer and electronics. The wound contact layer and cover layer can be sealed at a perimeter enclosing the dressing components.

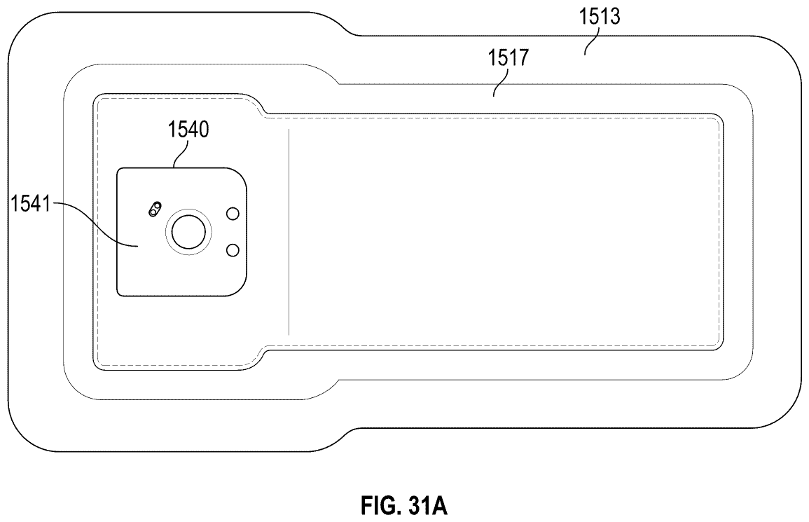

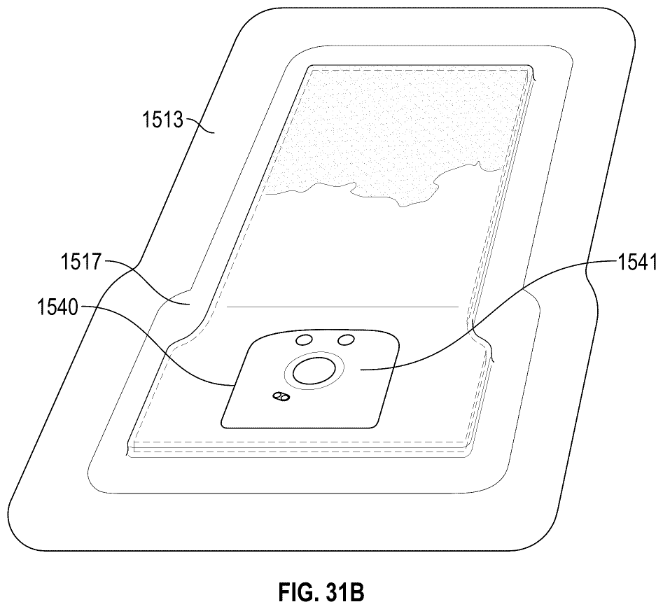

The wound dressing of FIGS. 31A-31B include an overlay layer 1517 comprising an additional layer of material positioned above the dressing layers. In some embodiments, the additional layer can include a masking or obscuring layer positioned above the dressing layers. The overlay layer 1517 can be positioned above the absorbent layer and electronics and below the cover layer 1513. In some embodiments, the overlay layer 1517 can include an aperture 1540 over a portion of the electronic components to allow the electronic components to be accessible from above the overlay layer. In some embodiments, the overlay layer 1517 can be an opaque material that does not allow the wound exudate or other fluid to be visible from a top view of the wound dressing. In some embodiments, the overlay layer can be an absorbent or transmission layer as described herein. In some embodiments, the overlay layer can comprise a conformable material overlaying and overbordering the perimeter of the lower layers of transmission and absorbent materials so as to protect the cover layer from being punctured by the lower layers when sealed over the dressing layers as described in more details below.

The wound dressing can include an electronics label or covering 1541 positioned over the aperture 1540 in the overlay layer 1517. In some embodiments, the label or covering 1541 can be positioned under the cover layer 1513. In other embodiments, the cover layer 1513 can be positioned below the label and can also have an aperture to allow the label or covering 1541 to communicate with the underlying electronic components.

FIG. 31B illustrate the wound dressing of FIG. 31A absorbing and retaining fluids while negative pressure is applied to the dressing.

FIGS. 31A-31B illustrate a label or covering 1541 that can be positioned over and cover the electronics and an opening 1540 in the overlay layer 1517

FIG. 32 illustrates an embodiment of wound dressing layers incorporating the electronic components within the wound dressing. FIG. 32 illustrates a wound dressing with a wound contact layer 1610 configured to contact the wound. A transmission layer or spacer layer 1611 is provided over the wound contact layer 1610. The transmission layer 1611 can assist in transmitting and distributing negative pressure over the wound site.

A first layer of apertured absorbent material 1651 can be provided over the transmission layer 1611. The first apertured absorbent layer 1651 can include an aperture 1629. In some embodiments, the aperture 1629 can be sized and shaped to fit the electronics unit 1650 therein. The first apertured absorbent layer 1651 can be sized and shaped to the size of the electronics area and does not extend into the absorbent area. In some embodiments, the apertures 1629 can be shaped and sized to fit the individual components of the electronics unit 1650.

A second apertured absorbent layer 1622 can be provided over the first absorbent layer 1651. In some embodiments, the second absorbent layer 1622 include apertures 1628. The second absorbent layer 1622 can be sized and shaped to the size of the electronics area and absorbent area. In some embodiments, the apertures 1628 can be shaped and sized to fit the individual components of the electronics unit 1650.

An electronics unit 1650 can be positioned in the apertures 1628 and 1629 of the first and second apertured absorbent material 1651 and 1622. The electronics unit 1650 can include a pump 1627, power source 1626, and a printed circuit board 1681. In some embodiments, the pump 1627 can include a pump inlet mechanism 1709 and an outlet mechanism 1682. In some embodiments, the printed circuit board 1681 can include electronics including but not limited to a switch, sensors, and LEDs as described herein. In some embodiments, the circuit board 1681 can include one or more hole to be positioned over one or more exhaust vents (not shown) in the outlet mechanism 1682 as described in more detail herein.

An overlay layer 1617 can be provided over the electronics components 1650 and absorbent layer 1622. In some embodiments, the overlay layer 1617 can be one or more layers of absorbent and/or transmission material as described herein. In some embodiments, the overlay layer 1617 can comprise a conformable material overlaying and overbordering the perimeter of the lower layers of transmission and absorbent materials. In some embodiments, the overlay layer 1617 can soften the edges of the wound dressing layers by decreasing the profile around the edges of the dressing layers. In some embodiments, the overlay layer 1617 can be provided to protect the cover layer from being punctured by the lower layers when positioned over the dressing layers as described in more details below. The overlay layer 1617 can include an aperture 1671 to allow access to at least a portion of the electronics unit 1650 positioned below.

A cover layer or backing layer 1613 can be positioned over the overlay layer 1617. In some embodiments, when the overlay layer 1617 is not used, the cover layer or backing layer 1613 can be provided above absorbent layers 1622, and/or electronic components 1650. The cover layer 1613 can form a seal to the wound contact layer 1610 at a perimeter region enclosing the overlay layer 1617, absorbent layers 1622 and 1651, electronic components 1650, and the transmission layer 1611. In some embodiments, the cover layer 1613 can be a flexible sheet of material that forms and molds around the dressing components when they are applied to the wound. In other embodiments, the cover layer 1613 can be a material that is preformed or premolded to fit around the dressing components. As used herein, the terms cover layer and backing layer can be used interchangeably to refer to the layer of material in the dressing configured to cover the layers of the wound dressing.

In some embodiments, the cover layer or backing layer 1613 can include an aperture 1672. The aperture 1672 can be positioned over at least a portion of the aperture 1671 in the overlay layer 1617 to allow access to at least a portion of the electronics unit 1650 positioned below. In some embodiments, the apertures 1671 and 1672 can allow access to the switch and/or venting holes of the pump exhaust.

A label 1641 can be provided over the apertures 1671 and 1672 and positioned over the exposed portion of the electronic components 1650. The label can include the vent holes 1642, indicator portions 1644, and/or switch cover 1643. The indicator portions 1644 can include holes or transparent regions 1644 for positioning over the one or more indicators or LEDs on the printed circuit board 1681 below the label 1641. The holes or transparent regions 1644 can allow for the indicators or LEDs to be visible through the label 1641. In some embodiments, the switch cover 1642 can include a dome shaped cover positioned over the switch on the printed circuit board 1681. In some embodiments, the label 1641 can include embossed features for the switch cover 1642. In some embodiments, the embossed features of the switch cover 1642 can prevent accidental activation or deactivation of the device. In some embodiments, the switch or switch cover 1642 can include a tab on the switch to prevent accidental activation or deactivation. The vent holes 1642 of the label can allow exhaust from the pump outlet mechanism to pass through the label and exit the wound dressing to be exhausted to the atmosphere.

In some embodiments, the label can be positioned on top of the cover layer or backing layer 1613. The label can be sealed to the top surface of the cover layer. In other embodiments, the label 1641 can be positioned above the overlay layer 1671 and below the cover layer or backing layer 1613. In such embodiments, the cover layer 1613 can have one or more apertures over one or more components of the label 1641. For example, the cover layer 1613 can have apertures over the vent holes 1642, indicator portions 1644, and/or switch cover 1643.

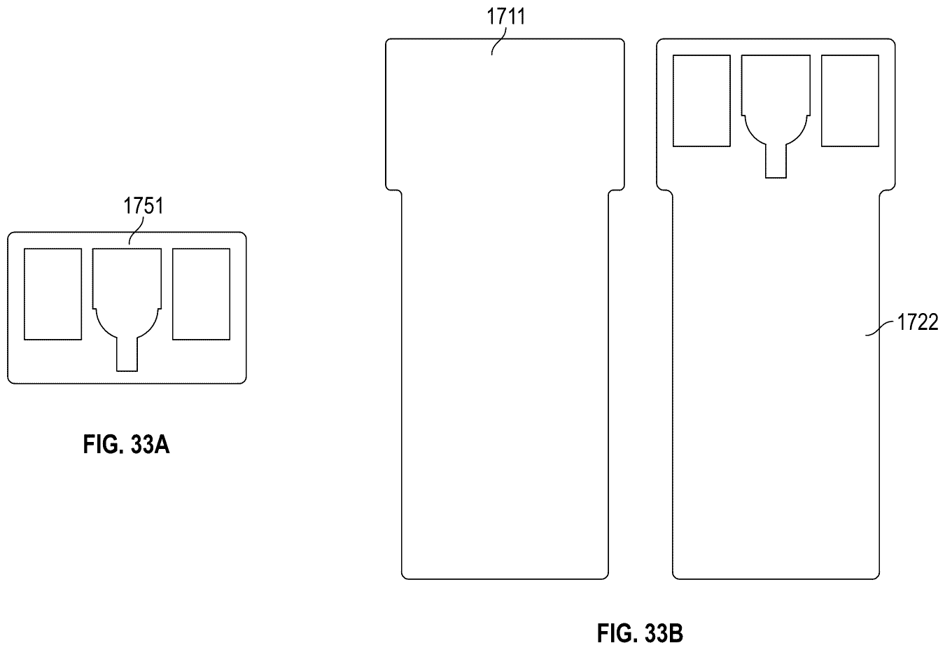

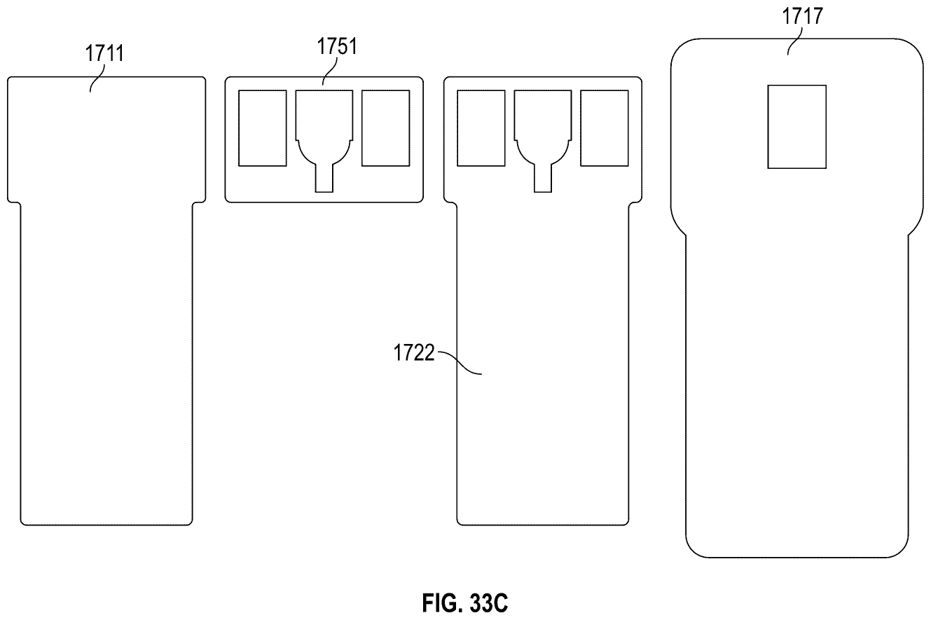

FIGS. 33A-33C illustrates the individual layers of a wound dressing. FIG. 33A illustrates a first apertured absorbent material 1751 cut to fit the size and shape of the electronics area.

FIG. 33B illustrates a second apertured absorbent layer 1722 and a transmission layer 1711. Both the second absorbent layer 1722 and transmission layer 1711 can be a similar size and shape as shown in FIG. 33B. The first apertured absorbent material 1751 can be a smaller apertured absorbent material than the size of the second apertured absorbent layer 1722.

FIG. 33C illustrates a transmission layer 1711, a first apertured absorbent layer 1751, a second apertured absorbent layer 1722, and overlay layer 1717. As shown in FIG. 33C, the overlay layer 1717 can have a larger perimeter size than the other layers of the dressing as to overhang the edges of the other layers of the wound dressing. In some embodiments, the overlay layer 1717 can have a smaller thickness than the absorbent layer 1722 and transmission layer 1711. In other embodiments, the overlay layer 1717 can have the same thickness or a greater thickness than the absorbent layer 1722 and transmission layer 1711.

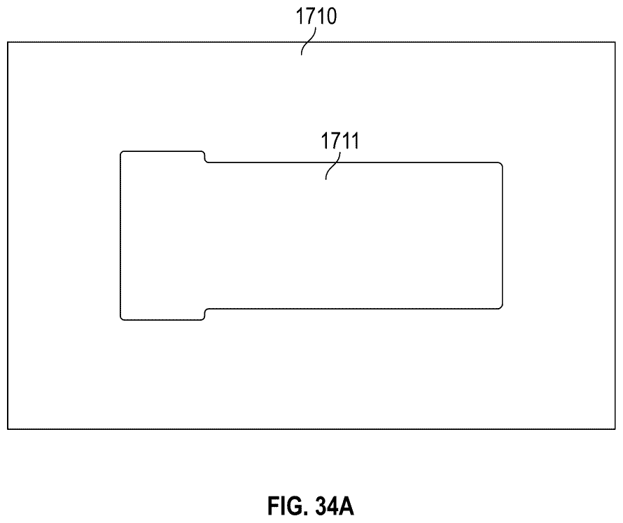

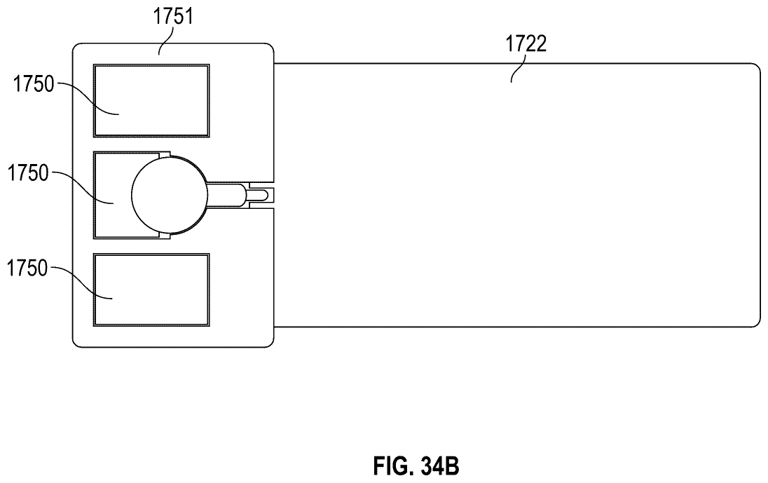

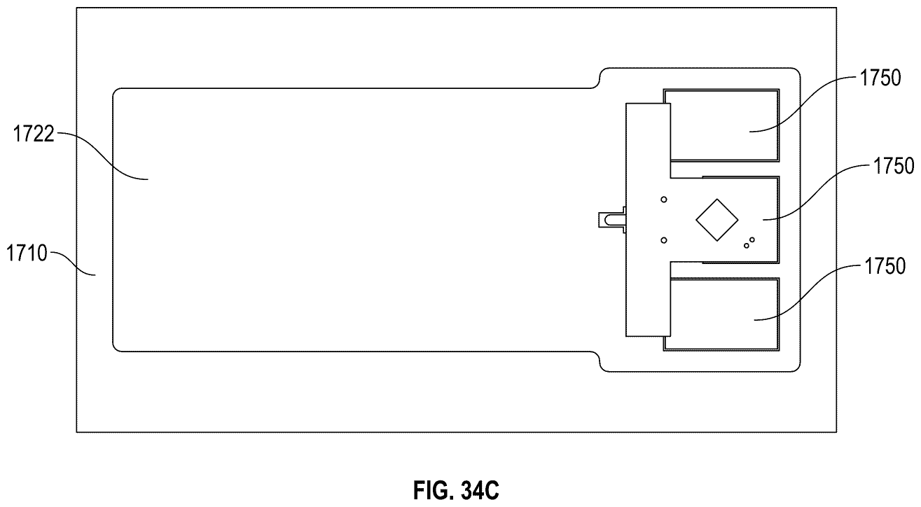

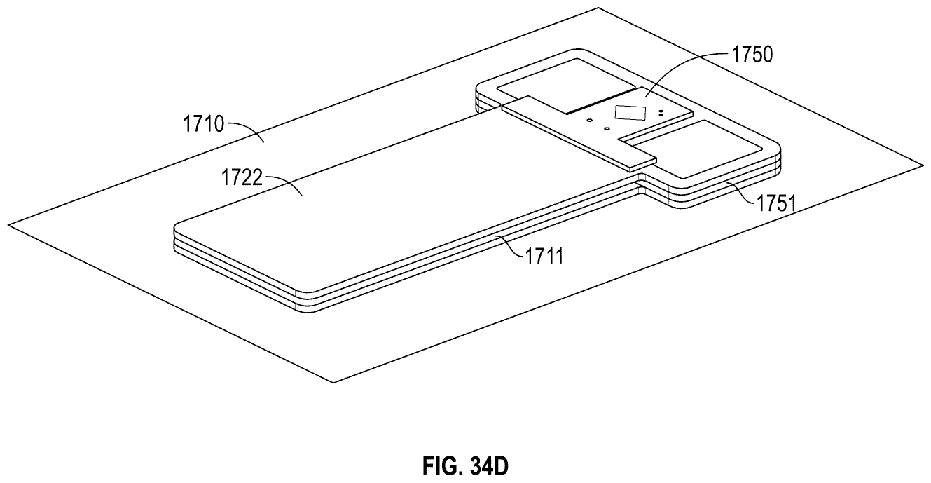

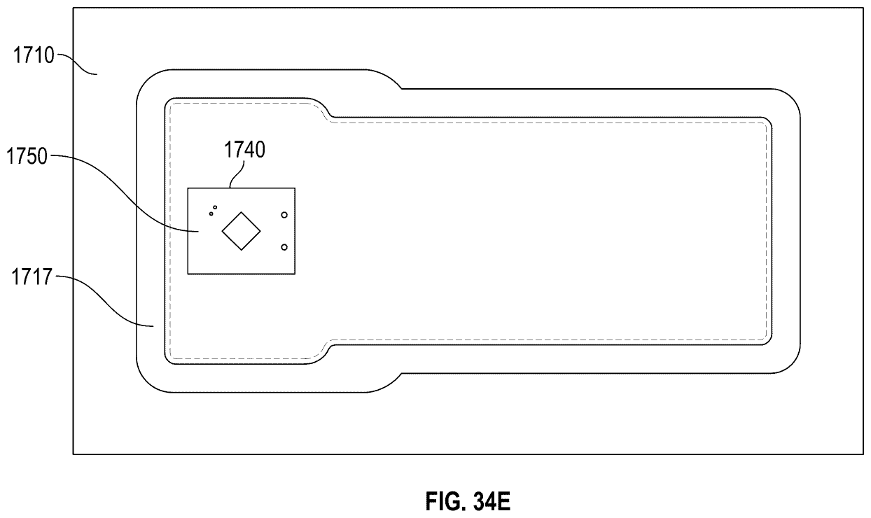

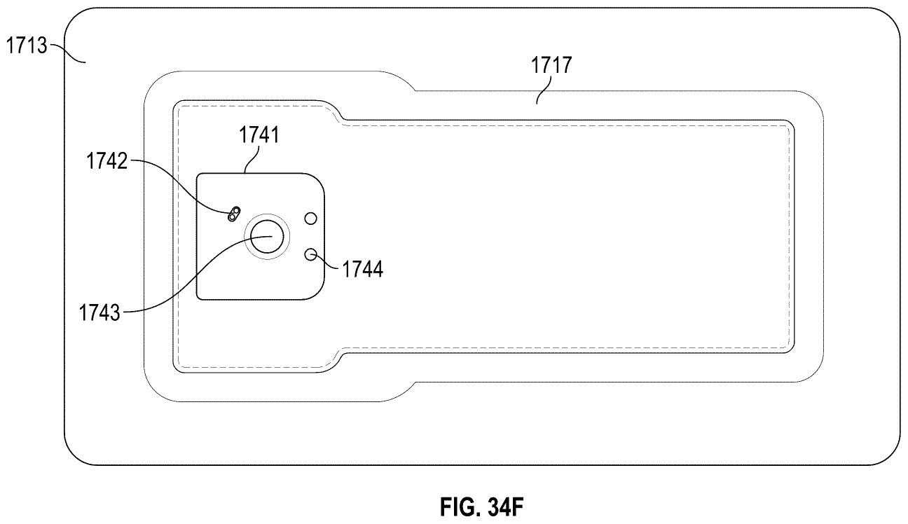

FIGS. 34A-34F illustrates the layers of the wound dressing incorporating an electronics assembly within the dressing. As shown in FIG. 34A, a transmission layer 1711 can be placed over a wound contact layer 1710. FIG. 34B illustrates a bottom view of components of the wound dressing. FIG. 34B illustrates the bottom view of an electronic unit 1750 embedded within the apertures of the first apertured absorbent layer 1751 and the second apertured absorbent layer 1722. FIG. 34C illustrates a top view of an electronics unit 1750 embedded within the apertures of the first apertured absorbent layer 1751 (not shown) and the second apertured absorbent layer 1722 placed over the transmission layer (not shown) and the wound contact layer 1710.

FIG. 34D illustrates the layers of the wound dressing device with the electronics unit 1750 embedded within the first apertured absorbent layer 1751 and the second apertured absorbent layer 1722. The first apertured absorbent layer 1751 and the second apertured absorbent layer 1722 can be placed over the transmission layer 1711 and the wound contact layer 1710.

FIG. 34E illustrates an overlay layer 1717 positioned over the dressing layers. The overlay layer 1717 includes an opening or aperture 1740 positioned over a portion of the electronics unit 1750. The aperture 1740 can allow for access to the switch, pump outlet components, and visual indicators on the top surface of the electronics unit 1750.

A label or covering 1741 can be positioned over and cover the electronics and an opening 1740 in the overlay layer 1717 as shown in FIG. 34F. FIG. 34F shows a cover layer 1713 covering the overlay layer 1717 and electronics covering 1741 and underlying dressing and electronics components. The cover layer 1713 can seal to the wound contact layer 1710 (shown in FIG. 34C-34E) at a perimeter region of the wound contact layer 1710. In some embodiments, the label or electronics covering 1741 can be positioned over the cover layer 1713. In some embodiments, the cover layer 1713 can seal over the electronics covering 1741. In some embodiments, the electronics covering 1741 can include a switch cover 1743, one or more visual indicators 1744, and/or pump outlet vent(s) 1742 as shown in FIG. 34F. In some embodiments, the cover layer 1713 can include one or more holes in the cover layer 1713 positioned over the switch and/or pump outlet vent(s). In some embodiments, the cover layer 1713 can include a single hole that is positioned over the switch cover 1743, visual indicators 1744, and/or pump outlet vent(s) 1742 in the covering or label 1741 as shown in FIG. 34F. In some embodiments, the label can include embossed features for the switch cover 1743. In some embodiments, the embossed features of the switch cover 1743 can prevent accidental activation or deactivation of the device. In some embodiments, the switch or switch cover 1743 can include a tab on the switch to prevent accidental activation or deactivation.

The visual indicators 1744 can provide an indication of operation of the negative pressure source and/or an indication of the level of negative pressure that is applied to the wound. In some embodiments, the visual indicators can include one or more light sources or LEDs. In some embodiments, the visual indicator light sources an illuminate to indicate a condition or change of condition. In some embodiments, the light source can illuminate in a particular sequence and/or color that indicates a condition. For example, in some embodiments, the light source can flash to notify the user that the device is operating properly. In some embodiments, the light source can automatically flash periodically and/or the light source can be activated by the switch or other button to light up and indicate a condition.

In some embodiments, the switch can be pressed and/or held down to power the dressing and electronics on and off. In some embodiments, once the switch is activated and the pump and associated colored LED, for example, green colored LED, can be used to conformed the dressing and integrated negative pressure source is operational. In some embodiments, during operation of the pump and dressing, the pump and dressing can enter the fault state indicated by a colored LED, for example, orange colored LED.

The electronics components can be incorporated in the dressing. For example, the dressing components can be assembled to form one integrated negative pressure dressing to be positioned over a wound. The following assembly description describes an embodiment of the assembly of an integrated wound dressing. In some embodiments, some or all of the assembly process can be automated and/or any or all of the processes or procedures can be done in any order.

A transmission layer can be positioned over the wound contact layer as shown in FIG. 34A. In some embodiments, the transmission layer can be positioned with the larger pores facing upward or away from the wound. FIG. 34B illustrates a bottom view of some of the components of the wound dressing to illustrate the electronic components embedded within or fit into the apertures of the large apertured pad or absorbent layer and small apertured pad or absorbent layer. In FIG. 34B, the electronics assembly is positioned switch side down. FIG. 34C illustrates the top view of the electronics assembly within the apertured pads or absorbent material placed directly on top of the transmission layer as shown in FIGS. 34C and 34D. The switch can be positioned on the top surface of the printed circuit board as shown in FIGS. 34C and 34D.

The overlay layer 1717 can be positioned over the apertured pads or absorbent material with the aperture in the overlay layer positioned over the switch of the electronics assembly. In some embodiments, the edges and/or the outer perimeter of the overlay layer 1717 can be adhered or secured to the top or upper surface of the wound contact layer 1710. A top film or cover layer can be placed over the overlay layer 1717 as shown in FIG. 34F. In some embodiments, the perimeter of the cover layer can be secured to the top or upper surface of the wound contact layer 1710. In some embodiments, if the cover layer is positioned over the printed circuit board, holes can be punctured in the top film at the location of the two exhaust ports. In other embodiments, the cover layer can be provided with one or more apertures that are placed over the two exhaust ports and/or other components of the electronics unit.

A label cover can be applied over the switch and/or other components of the electronics assembly that are exposed through the apertures of the overlay layer 1717 and the cover layer. The indicator portions can include transparent portions or LED windows aligned with the LED's on the PCB when the label cover is applied. In some embodiments, the LED windows can include apertures in the label cover. In other embodiments, the LED windows can be transparent portions of the label cover. The exhaust holes can also be aligned with apertures in the label cover.

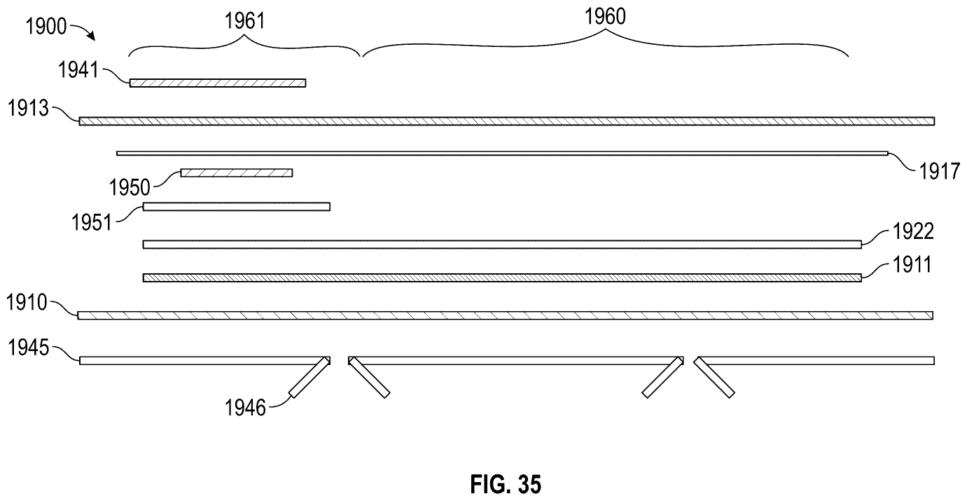

FIG. 35 illustrates a cross sectional layout of the material layers of the wound dressing incorporating an electronics assembly within the dressing. The dressing 1900 included multiple material layers and an electronics assembly 1950. The wound dressing 1900 can include an electronics area 1961 including the electronics and an absorbent area or dressing area 1960 that is intended to be applied to the wound as described with reference to FIGS. 27A-27B. As described herein, the one or more of the material layers can extend into both the electronics area 1961 and the dressing area 1960. The dressing 1900 can include a wound contact layer 1910, transmission layer 1911, absorbent layers 1922 and 1951, an overlay layer, and a cover or backing layer 1913 as illustrated in FIG. 35. The absorbent layers 1922 and 1951 can include recesses or cutouts to receive the components of the electronics assembly 1950 as described herein. In some embodiments, the overlay layer 1917 and/or the cover layer 1913 can include a cut out over the switch and/or indicators of the electronics assembly 1950. A label or covering 1941 can be positioned to cover at least a portion of the electronics assembly 1950 and any cutouts in the overlay layer 1917 and/or the cover layer 1913. The label or covering 1941 can be similar to the label or covering 1741 as described previously with reference to FIGS. 32 and 34F.

Before use, the dressing can include a delivery layer 1945 adhered to the bottom surface of the wound contact layer. The delivery layer 1945 can cover adhesive or apertures on the bottom surface of the wound contact layer 1910. In some embodiments, the delivery layer 1945 can provided support for the dressing and can assist in sterile and appropriate placement of the dressing over the wound and skin of the patient. The delivery layer 1945 can include handles 1946 that can be used by the user to separate the delivery layer 1945 from the wound contact layer 1910 before applying the dressing 1900 to a wound and skin of a patient.

### Electronics Unit

Disclosed embodiments relate to apparatuses and methods for wound treatment. A wound dressing apparatus can comprises a wound contact layer, at least one absorbent layer, an electronics unit comprising a negative pressure source unit, and a cover layer. The electronics unit can comprise a plurality of sensors positioned on a printed circuit board and an inlet protection mechanism of the negative pressure source unit comprises a first recess in fluid communication with a first sensor and the outlet or exhaust mechanism negative pressure source unit comprises a second recess in fluid communication with a second sensor.

The negative pressure wound therapy wound dressing described herein utilizes an embedded electronic circuit assembly to generate the negative pressure under the dressing. It can be important to protect the assembly from wound exudate or any other bodily fluid that would corrode the electronics. In addition, it can be important to protect the patient from the electric or electronic components. The assembly incorporates a pump which pulls air from the dressing to exhaust to the environment in order to produce the required negative pressure differential. Therefore, the means of protection of the electronics cannot be a complete encapsulation or potting of the assembly. The protection must allow movement of air from the dressing to the pump and exhausting the dressing to the environment. In addition, as a component of the electronic assembly, it is essential to protect the pump from bodily fluids. It can be helpful to provide a sealed electronics unit where components are protected from bodily fluids and environmental conditions and also allow for communication with the wound dressing layers and the external environment. Additionally, it can be useful to allow the electronic components integrated within the wound dressing to incorporate control circuitry and sensors to measure and determine negative pressure applied to the wound.

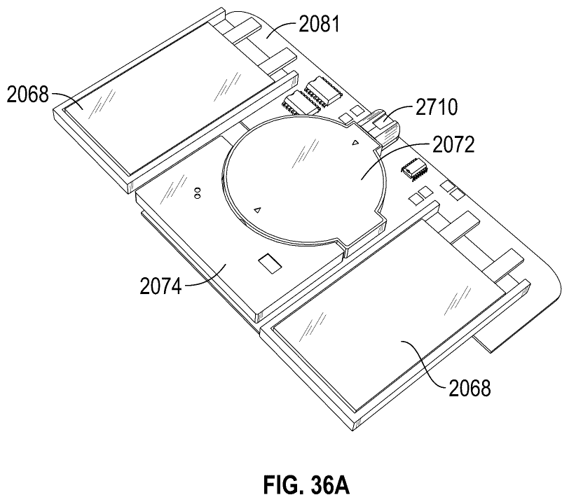

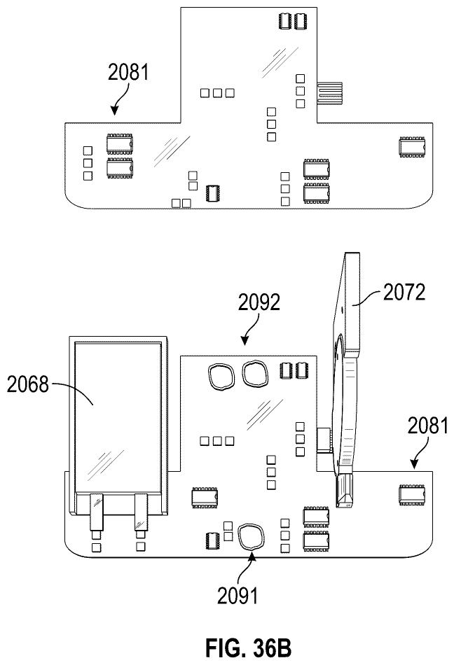

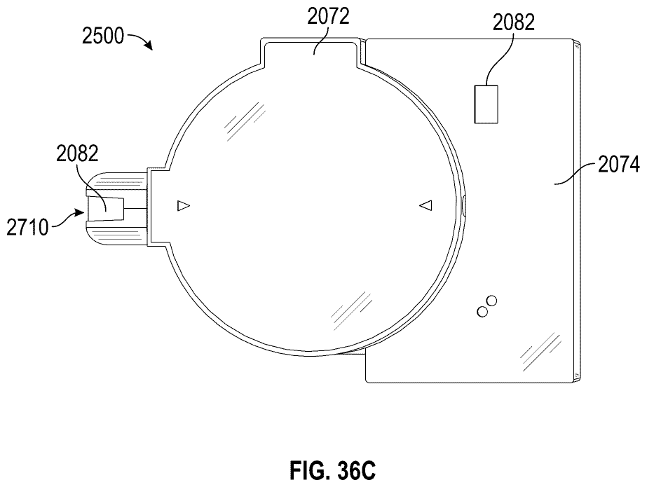

In some embodiments, the electronics unit requiring protection from the environment of the wound dressing can be partially encapsulated, potted or conformally coated. In some embodiments, the electronics unit can include a printed circuit board (PCB) 2081, the negative pressure source 2072, and one or more power sources 2068 as shown in FIGS. 36A and 36B. In some embodiments, the entirety of the electronics unit except for the pump inlet and pump outlet can be coated in a potted silicon enclosure. In some embodiments, potting of electronic components can include a process of filling a complete electronic assembly with a solid or gelatinous compound for resistance to shock and vibration, exclusion of moisture, and/or exclusion of corrosive agents.

FIGS. 36A-36C illustrates the pump assembly system 2500 with the pump inlet protection mechanism 2710 and pump outlet mechanism 2074 on the pump 2072. The pump assembly system 2500 can include cavities 2082 shown on the pump inlet protection mechanism 2710 and pump outlet mechanism 2074. In some embodiments, the inlet protection and pump outlet mechanisms can be adhered to the inlet and the outlet of the pump as described herein. In some embodiments, the pump assembly system 2500 can be assembled using an adhesive and allowed to cure prior to incorporating into the electronics assembly.

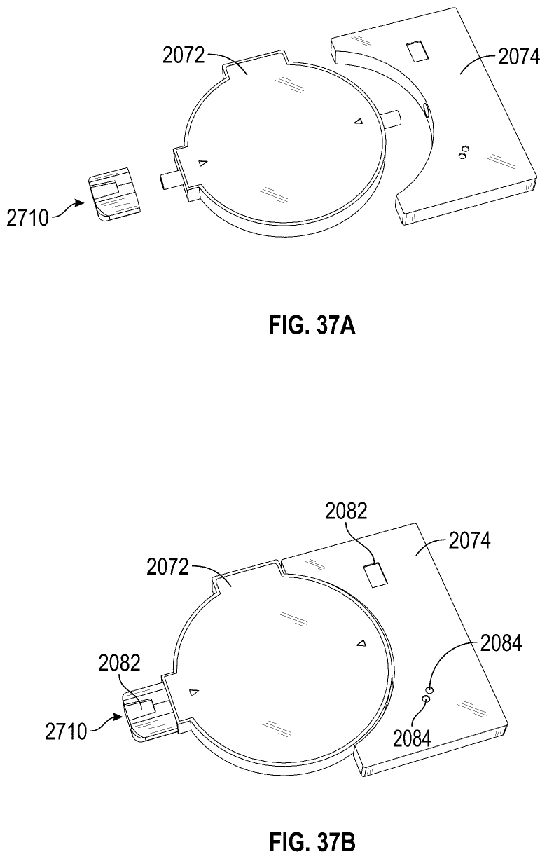

The pump inlet can be covered or fitted with a pump inlet protection mechanism 2710. In some embodiments, the pump inlet protection 2710 can be pushed onto the pump inlet as illustrated by the arrows in FIG. 37A. This can be a friction fit. The port of the pump inlet protection 2710 that receives a portion of the pump inlet can be sized and shaped to be a complementary fit around the pump inlet. In some embodiments, the pump inlet protection 2710 can be bonded onto the pump inlet using a silicone sealant or any other sealant or sealing technique. FIG. 37B illustrates the pump inlet protection mechanism 2710 covering the pump inlet and the pump outlet mechanism 2074 covering the pump outlet. The pump outlet mechanism 2074 can include vent holes 2084 to allow air exhausted from the pump to be exhausted from the pump outlet mechanism 2074. In some embodiments, the pump outlet mechanism 2074 can also include an exhaust vent hole 2084 in communication with a nonreturn valve and/or filter membrane of the pump outlet mechanism.

FIGS. 37A-37B illustrate a pump inlet protection mechanism 2710 and pump outlet mechanism 2074 with cavities 2082. The pump assembly including the pump inlet protection mechanism 2710 and pump outlet mechanism 2074 are placed over the surface of the printed circuit board 2081. When the pump assembly is in contact with the surface of the printed circuit board 2081, the cavities 2082 can be positioned over sensors on the printed circuit board 2081, for example, pressure sensors 2091 and 2092 on the printed circuit board 2081 illustrated in FIG. 36B. As used herein the terms pump exhaust mechanism and pump outlet mechanism can be used interchangeable to refer to the component or mechanism 2074 positioned on the outlet of the pump.

The pressure sensors can be used on the printed circuit board to measure and monitor the pressure levels produced by the pump as well as the pressure differential between the atmospheric pressure and the pressure underneath the wound dressing. FIG. 36B illustrates a first pressure sensor 2091 and a second pressure sensor 2092 on the printed circuit board 2081. The first pressure sensor 2091 can be used to monitor pressure underneath the wound dressing, such as pressure in a fluid flow path connecting the negative pressure source or pump 2072 and the wound, pressure at the wound, or pressure in the negative pressure source 2072. In some embodiments, the first pressure sensor 2091 can be in fluid communication with the cavity 2082 of the pump inlet protection mechanism 2710 shown in FIGS. 37A-37B.

The second pressure sensor 1092 can be used to monitor pressure external to the wound dressing. In some embodiments, the second pressure sensor 1092 can be in fluid communication with the cavity 1082 of the pump outlet mechanism 2074 shown in FIGS. 37A-37B. The pressure external to the wound dressing can be atmospheric pressure; however, the atmospheric pressure can vary depending on, for instance, an altitude of use or pressurized environment in which the TNP apparatus may be used.

The control circuitry of the PCB can control the supply of negative pressure by the negative pressure source 2072 according at least to a comparison between the pressure monitored by the first pressure sensor 2091 and the pressure monitored by the second pressure sensor 2092. In some embodiments, the control circuitry can vary the sampling rate at which pressures monitored by the first and second pressure sensors 2091 and 2092 are sampled, such as based at least on an amount of energy stored in the power source 2068 or whether the negative pressure source 2072 is supplying negative pressure. The sampling rate can be varied, for instance, to increase an amount of power consumed (that is, by increasing the sampling rate) or decrease an amount of power consumed (that is, by decreasing the sampling rate) by the control circuitry. A controller of the control circuitry can enter a sleep mode, which may be a mode during which the pressure monitored by the first and second pressure sensors 2091 and 2092 is not sampled, and the controller can vary the sampling rate by entering the sleep mode. The sleep mode may be a mode from which the controller can be awoken via a hardware or software interrupt. Embodiments of the wound dressings, wound treatment apparatuses and methods described herein may also be used in combination or in addition to those described in more detail with reference to PCT International Application No. PCT/EP2017/060464, filed May 3, 2017, titled NEGATIVE PRESSURE WOUND THERAPY DEVICE ACTIVATION AND CONTROL, which is hereby incorporated by reference in its entirety herein.

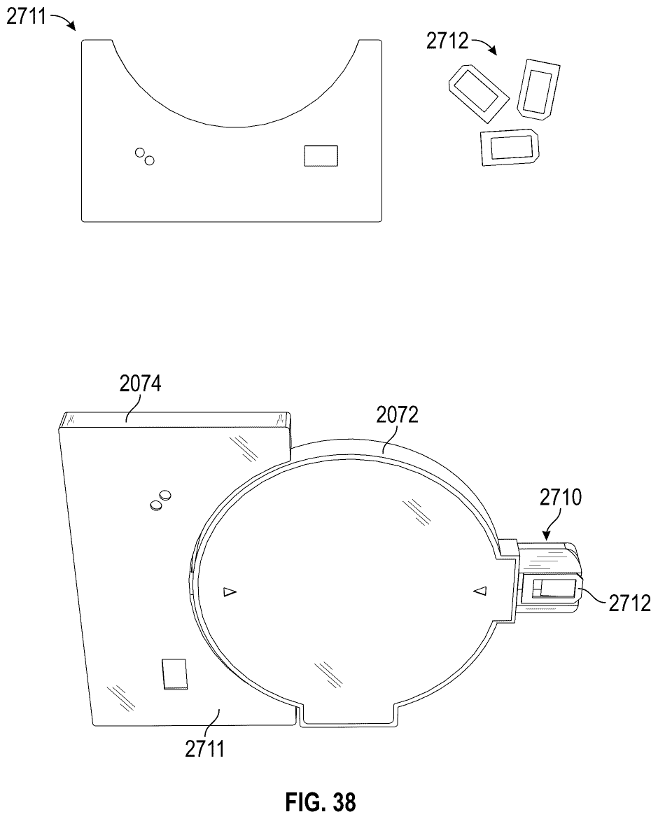

In some embodiments, a self-adhesive gasket 2711 and 2712 can be applied to the pump inlet protection 2710 and pump exhaust mechanism 2074 that seals the cavities 2082 of the pump inlet and pump exhaust around sensors on the printed circuit board 2081 and to seal around the exhaust mechanism vent holes and corresponding vent holes in the printed circuit board as illustrated in FIG. 38. In some embodiments, a pre-formed adhesive sheet can be used to form the sealing gaskets between the cavities 2082 of the pump inlet and pump exhaust mechanisms and sensors on the printed circuit board 2081 and between the exhaust mechanism vent holes and vent holes in the printed circuit board. In other embodiments, an adhesive can be used to seal the cavities 2082 of the pump inlet protection 2710 and pump exhaust mechanism 2074 around sensors on the printed circuit board 1081 and to seal around the exhaust mechanism vent holes 2084 and corresponding vent holes in the printed circuit board. FIG. 36B illustrates an embodiment of adhesive applied to the printed circuit board 2081 for adhering the pump inlet and pump exhaust mechanism to the printed circuit board 2081. In some embodiments, the electronics unit can be embedded within layers of the dressing in the electronics area 2361 as described previously. In some embodiments, the layers of the dressing in the electronics area 2361 can include cutouts or recesses into which the electronics unit can be placed.

The pump inlet component can be used to protect the pump from fluid on the inlet and the pump outlet mechanism can include a non-return valve that protects fluid from entering the outlet as described in more detail with reference to PCT International Application No. PCT/EP2017/055225, filed Mar. 6, 2017, titled WOUND TREATMENT APPARATUSES AND METHODS WITH NEGATIVE PRESSURE SOURCE INTEGRATED INTO WOUND DRESSING and PCT International Application No. PCT/EP2017/059883, filed Apr. 26, 2017, titled WOUND DRESSINGS AND METHODS OF USE WITH INTEGRATED NEGATIVE PRESSURE SOURCE HAVING A FLUID INGRESS INHIBITION COMPONENT, which are hereby incorporated by reference in their entireties herein.

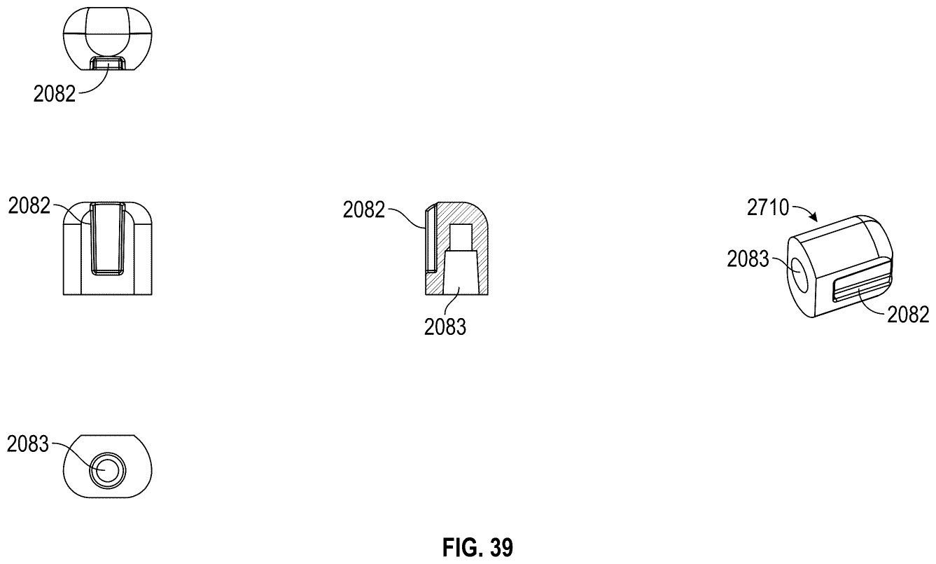

In some embodiments, the pump inlet protection mechanism 2710 can have a cavity 2082 to surround the sensor or other feature on the printed circuit board as shown in FIG. 39. The pump inlet protection mechanism can provide a large surface area available for vacuum to be drawn by the inlet of the pump. The pump inlet can fit within the recess 2083 in the pump inlet protection mechanism 2710 illustrated in FIG. 39. The pump inlet can be friction fit and/or form a complementary fit with the recess 2083 of the pump inlet protection mechanism as described herein. The pump inlet protection mechanism can have a rounded beveled shape or any other shape. The pump inlet protection mechanism can be formed from a porous material that allows for air or gas to pass through and can comprise one or more porous polymer molded components. In some embodiments, the pump inlet protection mechanism can be hydrophobic. In some embodiments, the pump inlet protection mechanism can have a pore size in the range of approximately 5 microns to approximately 40 microns. In some embodiments, the pore size can be approximately 10 microns. In some embodiments, the polymer can be one of hydrophobic polyethylene or hydrophobic polypropylene. In some embodiments, the pump inlet protection mechanism can be formed from a Porvair Vyon material with a pore size of 10 microns.

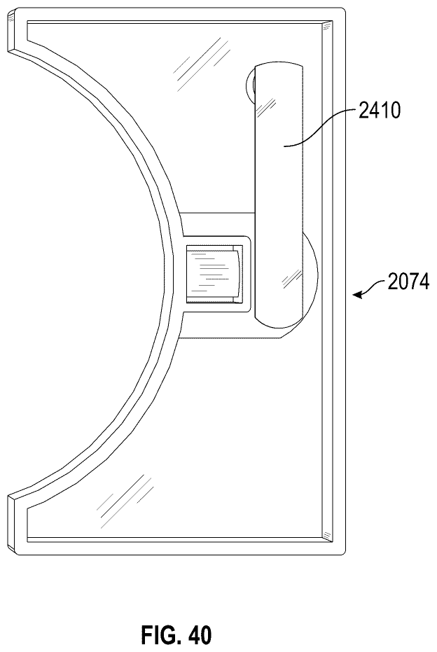

In some embodiments, the pump outlet mechanism can include a non-return valve as shown in FIG. 40. In some embodiments, the pump outlet mechanism can be bonded to the outlet of the pump using a sealant, for example a silicone sealant. The non-return valve can be similar to non-return valve described in PCT International Application No. PCT/EP2017/055225 incorporated by reference herein. As illustrated in FIG. 40, the non-return valve 2410 can include a reed valve or loose leaf valve. In some embodiments, the non-return valve 2410 can be any suitable mechanical one-way valve, such as, for example, a reed valve, a duckbill valve, a ball valve, or an umbrella valve, among others. In some embodiments, the outlet or exhaust of the pump outlet mechanism can include an antimicrobial film and/or other filter membrane. In some embodiments, the antimicrobial film or membrane can filter the air exhausted from the pump to the atmosphere.

In some embodiments, the pressure sensor(s) positioned on the printed circuit board aligned with the cavity of the pump inlet protection mechanism can be used to measure a pressure at the inlet of the pump or the inlet air flow. In some embodiments, the pressure sensor(s) positioned on the printed circuit board aligned with the cavity of the pump outlet mechanism can be used to measure a pressure at the outlet of the pump or the atmospheric air.

In some embodiments, software can be loaded onto the PCB. In some embodiments, the pump assembly 2500 including the pump inlet protection mechanism and the pump outlet mechanism can be soldered, adhered, or otherwise attached to the PCB. When self-adhesive gaskets are used between the pump assembly and PCB, the release liner can be pulled from the gaskets and the pump assembly can be pressed to the PCB to form a seal. In some embodiments, as illustrated in FIG. 36B, a thin bead of gasket sealant can be applied around the two pressure sensors and the exhaust aperture in the PCB and the pump assembly can be pressed to the PCB to form a seal. In some embodiments, both self-adhesive gaskets and the gasket sealant can be used. In some embodiments, the device utilizes either the self-adhesive gaskets or the gasket sealant.

The batteries can be soldered or attached to the PBC ensuring the terminals are in the correct orientation as illustrated in FIG. 36A. In some embodiments, a piece of foam or self-adhesive foam tape can be applied to the face of one or more batteries.

In some embodiments, a metal clicker dome can be fixed at the switch position on the PCB. In some embodiments, the clicker dome can be fixed or sealed to the PCB using an adhesive.

In some embodiment, a conformal coating can be applied to the electronics sub assembly to provide electrical and mechanical isolation of the electronics from the patient, components of the wound dressing, and exudate in the wound dressing. In some embodiments, liberal silicone coating can be applied to the electronics sub assembly. In some embodiments, the switch and LED's can remain uncoated. In some embodiments, the PCB edges can be coated. The coated assembly can be cured and inspected to confirm there are no defects in the coating. In some embodiments, more than one coating can be applied to the assembly to confirm coating of all the appropriate components.

The assembled electronics components can be incorporated in the dressing as described above. For example, the dressing components can be assembled to form one integrated negative pressure dressing to be positioned over a wound.

In some embodiments, the label and electronic components can be designed to provide mechanical and electrical isolation from the other areas of the dressing and from the patient. In some embodiments, the electrical isolation can be formed providing distance between the electronic components and the label cover. In some embodiments, electrical isolation can be provided through the coating and/or encapsulating the pump or other electrical components with an electrical isolating material. The electrical isolating material can include, but is not limited to, paint, foil, encapsulation with a conformable coating, and/or any other conductive material. In some embodiments, the electrical components can be encapsulated or coated with titanium to electrical isolate the electronic components. In some embodiments, the label can be formed from, coated with, or covered with the electrical isolating material. For example, the bottom surface of the label, in communication with the electronic components can be coated or covered with the electrical isolating material. In some embodiments, the top and/or bottom of the electrical components and/or label can be coated or covered with the electrical isolating material. The wound dressing with integrated electronic components as described herein can be defibrillation proof.

The wound dressing with embedded electronics can be any shape or size to accommodate various types of wounds. For example, the wound dressing with embedded electronics can have a rectangular, rounded rectangular, square, T shaped, or any other shape or design. In some embodiments, the wound dressings with embedded electronics described herein can be rectangular or rounded rectangular shaped as illustrated with reference to FIGS. 27A-27B. In other embodiments, the wound dressings with embedded electronics described herein can be a T shaped as illustrated with reference to FIGS. 30A-30F.

In some embodiments, the absorbent components and electronics components can be overlapping but offset. For example, a portion of the electronics area can overlap the absorbent area, for example overlapping the superabsorber layer, but the electronics area is not completely over the absorbent area. Therefore, a portion of the electronics area can be offset from the absorbent area and only provided over the cushioning spacer layers.

All of the features disclosed in this specification (including any accompanying exhibits, claims, abstract and drawings), and/or all of the steps of any method or process so disclosed, may be combined in any combination, except combinations where at least some of such features and/or steps are mutually exclusive. The disclosure is not restricted to the details of any foregoing embodiments. The disclosure extends to any novel one, or any novel combination, of the features disclosed in this specification (including any accompanying claims, abstract and drawings), or to any novel one, or any novel combination, of the steps of any method or process so disclosed.

Conditional language, such as “can,” “could,” “might,” or “may,” unless specifically stated otherwise, or otherwise understood within the context as used, is generally intended to convey that certain embodiments include, while other embodiments do not include, certain features, elements, or steps. Thus, such conditional language is not generally intended to imply that features, elements, or steps are in any way required for one or more embodiments or that one or more embodiments necessarily include logic for deciding, with or without user input or prompting, whether these features, elements, or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Likewise the term “and/or” in reference to a list of two or more items, covers all of the following interpretations of the word: any one of the items in the list, all of the items in the list, and any combination of the items in the list. Further, the term “each,” as used herein, in addition to having its ordinary meaning, can mean any subset of a set of elements to which the term “each” is applied. Additionally, the words “herein,” “above,” “below,” and words of similar import, when used in this application, refer to this application as a whole and not to any particular portions of this application.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

Various modifications to the implementations described in this disclosure may be readily apparent to those skilled in the art, and the generic principles defined herein may be applied to other implementations without departing from the spirit or scope of this disclosure. Thus, the disclosure is not intended to be limited to the implementations shown herein, but is to be accorded the widest scope consistent with the principles and features disclosed herein. Certain embodiments of the disclosure are encompassed in the claim set listed below or presented in the future.

1.-63. (canceled)

64. A wound dressing apparatus comprising:
a wound contact layer configured to be positioned in contact with a wound;
a first area over the wound contact layer comprising:
a spacer layer; and
an absorbent layer over the spacer layer;
a second area over the wound contact layer comprising an electronics unit, the electronics unit comprising a negative pressure source and/or electronic components, wherein the first area is positioned adjacent to the second area;
wherein the second area comprises a cradle configured to allow fluid communication between the first area and second area, wherein the cradle comprises recesses configured to receive a portion of the electronics unit; and
a cover layer configured to cover and form a seal over the wound contact layer, the first area, and the second area.

65. The wound dressing apparatus of claim 64, wherein the cradle comprises a hydrophilic material.

66. The wound dressing apparatus of claim 64, wherein the cradle comprises a recess configured to receive a portion of the absorbent layer.

67. The wound dressing apparatus of claim 64, wherein the electronic components comprise one or more of a power source, a flexible circuit board, a sensor, a switch, and/or a light or LED indicator.

68. The wound dressing apparatus of claim 64, further comprising a negative pressure source inlet protection mechanism and a negative pressure source outlet or exhaust.

69. The wound dressing apparatus of claim 68, wherein the negative pressure source outlet or exhaust comprises an antibacterial membrane and/or a non-return valve.

70. The wound dressing apparatus of claim 68, wherein the cover layer comprises an aperture over the negative pressure source outlet or exhaust.

71. The wound dressing apparatus of claim 68, wherein the negative pressure source inlet protection mechanism comprises a hydrophobic material configured to prevent fluid from entering the negative pressure source.

72. A wound dressing apparatus comprising:
a wound contact layer comprising a proximal wound-facing face and a distal face, wherein the proximal wound-facing face is configured to be positioned in contact with a wound;
a spacer layer comprising a proximal wound-facing face and a distal face, the spacer layer positioned over the distal face of the wound contact layer;
a first area over the spacer layer comprising an absorbent layer;
a second area over the spacer layer comprising an electronics unit, the electronics unit comprising a negative pressure source and/or electronic components, wherein the first area is positioned adjacent to the second area;
wherein the second area over the spacer layer comprises a cradle configured to allow fluid communication between the first area and second area, wherein the cradle comprises recesses configured to receive the electronics unit; and
a cover layer configured to cover and form a seal over the wound contact layer, the spacer layer, the first area, and the second area.

73. The wound dressing apparatus of claim 72, wherein the electronic components comprise one or more of a power source, a flexible circuit board, a sensor, a switch, and/or a light or LED indicator.

74. The wound dressing apparatus of claim 72, further comprising a negative pressure source inlet protection mechanism and a negative pressure source outlet or exhaust.

75. The wound dressing apparatus of claim 74, wherein the negative pressure source outlet or exhaust comprises an antibacterial membrane and/or a non-return valve.

76. The wound dressing apparatus of claim 74, wherein the cover layer comprises an aperture over the negative pressure source outlet or exhaust.

77. The wound dressing apparatus of claim 74, wherein the negative pressure source inlet protection mechanism comprises a hydrophobic material configured to prevent fluid from entering the negative pressure source.

78. A wound dressing apparatus comprising:
a wound contact layer comprising a proximal wound-facing face and a distal face, wherein the proximal wound-facing face is configured to be positioned in contact with a wound;
a spacer layer comprising a proximal wound-facing face and a distal face, the spacer layer positioned over the distal face of the wound contact layer;
an absorbent layer comprising a proximal wound-facing face and a distal face, the absorbent layer positioned on the distal face of the spacer layer;
an electronics unit comprising a negative pressure source and/or electronic components;
a cradle positioned on the distal face of the absorbent layer and configured to allow fluid communication between the absorbent layer and the electronics unit, wherein the cradle comprises recesses configured to receive the electronics unit; and
a cover layer configured to cover and form a seal over the wound contact layer, the spacer layer, the absorbent layer, the cradle and the electronics unit.

79. The wound dressing apparatus of claim 78, wherein the cradle comprises a hydrophilic material.

80. The wound dressing apparatus of claim 78, further comprising a negative pressure source inlet protection mechanism and a negative pressure source outlet or exhaust.

81. The wound dressing apparatus of claim 80, wherein the negative pressure source outlet or exhaust comprises an antibacterial membrane and/or a non-return valve.

82. The wound dressing apparatus of claim 80, wherein the cover layer comprises an aperture over the negative pressure source outlet or exhaust.

83. The wound dressing apparatus of claim 80, wherein the negative pressure source inlet protection mechanism comprises a hydrophobic material configured to prevent fluid from entering the negative pressure source.
